(19) United States
(12) Patent Application Publication
(10) Pub. No.: US 2011/0189670 A1
(43) Pub. Date: Aug. 4, 2011

(54) Circulating Tumor and Tumor Stem Cell Detection Using Genomic Specific Probes

(76) Inventors: Ruth L Katz, Houston, TX (US); Abha Khanna, Sugar Land, TX (US); Tanweer M. Zaidi, Missouri City, TX (US); Weigong He, Sugar Land, TX (US); Ricardo L. Fernandez, Houston, TX (US); Ivan Gorlov, Houston, TX (US)

(21) Appl. No.: 13/002,944

(22) PCT Filed: Jul. 7, 2009
(86) PCT No.: PCT/US09/49845

§ 371 Date: Mar. 31, 2011

Related U.S. Application Data
(60) Provisional application No. 61/078,718, filed on Jul. 7, 2008.

(51) Int. Cl. C12Q 1/68

(52) U.S. Cl. 435/6.11

(57) ABSTRACT

The present invention comprises a method of detecting circular tumor cells and methods of detecting, evaluating, or staging cancer in a patient, as well as a method of monitoring treatment of cancer in a patient using the claimed method. The method comprises contacting a sample with a CD45 binding agent; selecting the cells based on positive or negative CD45 staining; contacting the selected cells with a labeled nucleic acid probe, and detecting hybridized cells by fluorescence in situ hybridization; and analyzing a signal produced by the labels on the hybridized cells to detect the CTCs. In other embodiments, the method provides for directed to a method of determining the level of CTCs in a sample having blood cells from a patient by contacting a sample having blood cells from a patient, wherein the sample has not been pre-sorted into CD45-positive and CD45-negative cells.

The present application claims benefit of priority to U.S. Provisional Application Ser. No. 61/078,718 filed Jul. 7, 2008, the entire contents of which are hereby incorporated by reference.

### BACKGROUND OF THE INVENTION

1. Field of the Invention

The present invention relates to the fields of oncology, genetics and molecular biology. More particularly, the invention relates to the use of probes for regions that are highly predictive of the development of neoplasia and progression of neoplastic events. Using this invention, subjects can be screened for, e.g., lung cancer using a minimal amount of blood (e.g., a finger prick).

2. Description of Related Art

In 2005, it is estimated that lung cancer accounted for 13% of new cancer cases and was the leading cause of cancer deaths in the United States. Unfortunately, the overall 5-year survival rate remains less than 15%, despite advances in treatment. Clearly, there is a need to develop novel strategies for treatment of lung cancer, and at the same time develop sensitive surrogate biomarkers that can serve to monitor early response to new therapies. The presence of circulating cancer cells (CTCs) or tumor stem cells that compose a small but vital part of the tumor subpopulation is presently considered to be the “holy grail” for detection and eradication for patient response and survival.

Cristofanilli et al. (2004), in a prospective study of patients with metastatic breast cancer, showed that patients whose CTCs were above 5 per 7.5 ml of blood at baseline were associated with both a significantly shorter progression-free survival and shorter overall survival. Pierga et al. similarly reported that the presence of cytokeratin positive CTCs in peripheral blood of patients with breast cancer corresponded with stage and prognosis (Pierga et al., 2004). Some investigators have looked at the genomic signatures in the metastasizing cells compared to the primary tumors and have found a gene expression signature in the primary tumor that predicts for metastasis and poor clinical outcome (Gangnus et al., 2004; Ramaswamy et al., 2003; Muller and Pantel, 2004). Others have used PCR to identify genes associated with CTCs in peripheral blood in non-small cell lung cancer (NSCLC) cases and have shown that poor therapeutic response was associated with detection of CTC after therapy (Sher et al., 2005).

A consensus is emerging that a crucial early event in carcinogenesis is the induction of the genomic instability phenotype, which enables an initiated cell to evolve into a cancer cell by achieving a greater proliferative capacity (Fenech et al., 2002). It is well known that cancer results from an accumulation of multiple genetic changes that can be mediated through chromosomal changes and therefore has the potential to be cytogenetically detectable (Solomon et al., 1991). It has been hypothesized that the level of genetic damage in peripheral blood lymphocytes reflects amount of damage in the precursor cells that lead to the carcinogenic process in target tissues (Hagmar et al., 1998). Evidence that cytogenetic biomarkers are positively correlated with cancer risk has been strongly validated in recent results from both cohort and nested case-control studies showing that chromosome aberrations as a marker of cancer risk (Liou et al., 1999; Bonassi et al., 2000; Bonassi et al., 2004; Smerhovsky et al., 2001; Tucker and Preston, 1996) reflecting both the genotoxic effects of carcinogens and individual cancer susceptibility commonly used methods for measuring DNA damage because it is relatively easier to score micronuclei (MN) than chromosome aberrations (Fenech et al., 2002). MN originates from chromosome fragments or whole chromosomes that fail to engage with the mitotic spindle and therefore lag behind when the cell divides.

Factors predicting clinical outcome in lung cancer patients include extent of disease or tumor burden. Circulating tumor cells (CTCs) may be a measure of tumor burden, and may also be a method to more accurately stage patients. Previously CTCs were isolated from whole blood based on assays employing magnetic beads coated with anti-cytokeratin antibodies (positive selection) or depletion of CD45 lymphoid cells with an antibody to keratin (EPICAM) for epithelial cells or depletion of CD45 cells. The OncoQuick system involves gradient separated cells and immunohistochemistry followed by image analysis. Previous methods to detect CTCs also include PCR-assays. However these cannot quantify number of tumor cells or look at morphology. It has been found that yields of circulating cancer cells have been low.

Compared to other cytogenetic assays, quantification of MN confer several advantages, including speed and ease of analysis, no requirement for metaphase cells and reliable identification of cells that have completed only one nuclear division, which prevents confounding effects caused by differences in cell division kinetics because expression of MN, NPBs or NBUDs is dependent on completion of nuclear division (Fenech, 2000). Because cells are blocked in the binucleated stage, it is also possible to measure nucleoplasmic bridges (NPBs) originating from asymmetrical chromosome rearrangements and/or telomere end fusions (Umegaki et al., 2000; Stewenius et al., 2005). NPBs occur when the centromeres of dicentric chromosomes or chromatids are pulled to the opposite poles of the cell at anaphase. In the CBMN assay, binucleated cells with NPBs are easily observed because cytokinesis is inhibited, preventing breakage of the anaphase bridges from which NPBs are derived, and thus the nuclear membrane forms around the NPB. Both MN and NPBs occur in cells exposed to DNA-breaking agents (Stewenius et al., 2005; Fenech and Crott, 2002) In addition to MN and NPBs, the CBMN assay allows for the detection of nuclear buds (NBUDs), which represent a mechanism by which cells remove amplified DNA and are therefore considered a marker of possible gene amplification (reviewed by Fenech (2002). The CBMN test is slowly replacing the analysis of chromosome aberrations in lymphocytes because MN, NPBs and NBUDs are easy to recognize and score and the results can be obtained in a shorter time (Fenech, 2002).

Thus, there is a need to develop methods for detecting CTCs and determining the level of CTCs in samples.

### SUMMARY OF THE INVENTION

In some embodiments, the invention is directed to a method of detecting circulating tumor cells (CTCs) in a sample comprising contacting said sample with a CD45 binding agent; selecting the cells based on staining for CD45; contacting the selected cells with a labeled nucleic acid probe, and detecting hybridized cells by fluorescence in situ hybridization; and analyzing a signal produced by the labels on the hybridized cells to detect the CTCs. The cells that are selected may show positive staining for CD45 or diminished or no staining for CD45.

The cells may be selected by any method known to those of skill in the art, including but not limited to standard cell detection techniques such as flow cytometry, cell sorting, automated flourescense scanning, immunocytochemistry (e.g., staining with tissue specific or cell-marker specific antibodies), fluorescence activated cell sorting (FACS), magnetic activated cell sorting (MACS), by examination of the morphology of cells using light or confocal microscopy or a bright field examination using chromogen labeled probes such as DAB or AEC, and/or by measuring changes in gene expression using techniques well known in the art, such as PCR and gene expression profiling. In a particular embodiment, the cells are selected by automated flourescense scanners.

In some embodiments, the staining comprises contacting the sample with a labeled CD45 antibody. The label may be any type of label known to those of skill in the art, including but not limited to a fluorescent label or a chromagen label. In some embodiments, the labeled CD45 is a fluorescently-labeled CD45 antibody. In particular embodiments, the fluorescently-labeled CD45 antibody is a Fluorescein isothiocyanate (FITC)-conjugated CD45 antibody.

The sample may be any biological sample that contains blood cells. Various embodiments include paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, colon, skin, stomach, spleen, lymph node, bone marrow or kidney. In some embodiments, the sample is a blood sample. In particular embodiments, the blood sample includes lympocytes, monocytes, neutrophils, stem cells, and circulating tumor cells. In particular embodiments, the blood sample is a buffy coat layer separated from the blood by a Ficoll-Hypaque gradient.

The signal may be detected by any method known to those of skill in the art. In particular embodiments, the signal is detected using an automated fluorescence scanner.

In some embodiments, the blood sample may be a human blood sample from a patient. The patient may be known or suspected to have cancer. The cancer may be any form of cancer that gives rise to blood borne metastases, including but not limited to cancer of the lung, breast, colon, prostate, pancreas, esophagus, kidney, gastro-intestinal tumors, urigenital tumors, kidney, melanomas, endocrine tumors, sarcomas, lymphoma, or leukemia.

The probes may be may be specific for any genetic marker that is most frequently amplified or deleted in CTCs. In particular, the probes may be a 3p22.1 probe, which is a nucleic acid probe targeting RPL14, CD39L3, PMGM, or GC20, combined with centromeric 3; a 10q22-23 probe (encompassing surfactant protein A1 and A2) combined with centromeric 10; or a PI3 kinase probe. Other genetic markers may include, but are not limited to, centromeric 3, 7, 17, 9p21, 5p15.2, EGFR, C-myc8q22, 6p22-22, CMET, HTTRT, and AP2β. In particular embodiments, the probe is a UroVysion DNA probe set (Vysis/Abbott Molecular, Des Plaines, Ill.), which includes probes directed to centromeric 3, centromeric 7, centromeric 17, 9p21.3. In other embodiments, the probe set is a LaVysion DNA probe set (Vysis/Abbott Molecular, Des Plaines, Ill.), which includes probes to 7p12 (epidermal growth factor receptor); 8q24.12-q24.13 (MYC); 6p11.1-q11 (chromosome enumeration (Probe CEP 6); and 5p15.2 (encompassing the SEMA5A gene). In still further embodiments, the probe may be a centromeric 7/7p12 Epidermal Growth Factor (EGFR) probe. The probe set may be a combination of any of the probes listed above or any probes known to those of skill in the art. In particular embodiments, the combination of probes is a cep10/10q22.3 and a cep3/3p22.1. In further embodiments, the combination of probes is cep7/7p22.1, a cep17, and a 9p21.3. In further embodiments, the combination of probes is cep10, 10q22.3 and EGFR. In further embodiments, the combination of probes is centromeric 3, 3p22.1, and 9p21.

In other embodiments, the invention is directed to a method of determining the level of circulating tumor cells (CTCs) in a sample having blood cells from a patient by contacting said sample with a CD45 binding agent; selecting the cells based on staining for CD45; contacting the selected cells with a labeled nucleic acid probe, and detecting hybridized cells by fluorescence in situ hybridization; and analyzing a signal produced by the labels on the hybridized cells to determine the level of CTCs in the sample. In other embodiments, the invention is directed to a method of determining the level of CTCs in a sample having blood cells from a patient by contacting a sample having blood cells from a patient, wherein the sample has not been pre-sorted into CD45-positive and CD45-negative cells.

In some embodiments, the method is directed to a method of detecting cancer in a patient comprising determining the level of CTCs in a biological sample containing blood cells from the patient by the described method, wherein the presence of CTCs in the sample is indicative of cancer. In particular embodiments, the sample is a blood sample which is obtained by a minimally-invasive procedure, such as a finger prick.

In some embodiments, a biological sample is obtained from a patient. In other embodiments of the method, the entity evaluating the sample for CTC levels did not directly obtain the sample from the patient. Therefore, methods of the invention involve obtaining the sample indirectly or directly from the patient. To achieve these methods, a doctor, medical practitioner, or their staff may obtain a biological sample for evaluation. The sample may be analyzed by the practitioner or their staff, or it may be sent to an outside or independent laboratory. The medical practitioner may be cognizant of whether the test is providing information regarding a quantitative level of CTCs.

In any of these circumstances, the medical practitioner may know the relevant information that will allow him or her to determine whether the patient can be diagnosed as having an aggressive form of cancer and/or a poor cancer prognosis based on the level of CTCs. It is contemplated that, for example, a laboratory conducts the test to determine the level of CTCs. Laboratory personnel may report back to the practitioner with the specific result of the test performed.

In still further embodiments, the invention concerns a method of evaluating cancer in a patient comprising determining the level of CTCs in a biological sample containing blood cells from the patient by the described method, wherein high levels of CTCs in the sample as compared to a control is indicative of an aggressive form of cancer and/or a poor cancer prognosis. The control may be any sample that has a known CTC level. In particular embodiments, the control is a non-cancerous sample. In still further embodiments, the invention concerns a method of identifying a patient at high risk to develop certain cancers based on genetic abnormality present in PBMCs even if the patient has not manifested overt evidence of cancer.

In yet further embodiments, the invention provides a method of monitoring treatment of cancer in a patient comprising determining the level of CTCs in a first sample from the patient by the disclosed method; determining the level of CTCs in a second sample from the patient after treatment is effected by the described method; and comparing the level of CTCs in the first sample with the level of CTCs in the second sample to assess a change and monitor treatment. In particular embodiments, the method further comprises treating the cancer based on whether the level of CTCs is high. The treatment may be any treatment known to those of skill in the art, including but not limited to chemotherapy, radiotherapy, surgery, gene therapy, immunotherapy, targeted therapy, or hormonal therapy.

In still further embodiments, the invention provides a method of staging cancer in a patient comprising determining the level of CTC expression in a biological sample containing blood cells from the patient by the described method, wherein a higher level of CTC in the sample as compared to a control is indicative of a more advanced stage of cancer and a lower level of CTC in the sample as compared to a control is indicative of a less advanced stage of cancer. The control may be any known sample, including but not limited to a non-cancerous sample, a cancer stage 0 sample, a cancer stage I sample, a lung cancer stage 1A sample, a lung cancer stage 1B sample, a cancer stage 11 sample, a cancer stage III sample, or a cancer stage 1V sample. In particular embodiments, the method is used to refine the staging of cancer after treatment has started. In particular embodiments, the level of CTCs is at least 50% more, compared to the level in a control sample. In other embodiments, the level of CTCs is at least about or at most about 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-fold or times, or any range derivable therein, greater than the level of a control sample. In particular embodiments, the level of CTCs is at least 2-fold greater than the level of a control sample.

In yet further embodiments, the invention provides a method of staging cancer in a patient comprising determining the level of CTC expression in a biological sample containing blood cells from the patient by the described method, wherein a higher or lower level of expression of a gene of interest in the sample as compared to a control is indicative of a more advanced stage of cancer and a lower level of expression of the gene of interest in the sample as compared to a control is indicative of a less advanced stage of cancer.

It is contemplated that any embodiment discussed in this specification can be implemented with respect to any method or composition of the invention, and vice versa. Furthermore, compositions of the invention can be used to achieve methods of the invention.

The use of the word “a” or “an” in the claims and/or the specification may mean “one,” but it is also consistent with the meaning of “one or more,” “at least one,” and “one or more than one.”

The phrase “one or more” as found in the claims and/or the specification is defined as 1, 2, 3, 4, 5, 6, 7, 8, 9, 10, or more.

Throughout this application, the terms “about” and “approximately” indicate that a value includes the inherent variation of error for the device, the method being employed to determine the value, or the variation that exists among the study subjects. In one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The invention may be better understood by reference to one or more of these drawings in combination with the detailed description of specific embodiments presented herein.

FIGS. 1A-C: Comparison of FISH between CD45-positive and CD45-negative cells. FIG. 1A indicates the ratio with the FISH (3p) unenriched specimen before staining with CD45 looking at peripheral blood mononuclear cells in a patient with lung cancer; FIG. 1B indicates the ratio with the FISH 3p (CD45-positive) cells; and FIG. 1C indicates the ratio with the FISH 3p CD45-negative cells.

FIG. 2: Location of 3p21.3 BAC Probe on Chromosome 3.

FIG. 3: Location of 10q22 BAC Probe on Chromosome 10.

FIG. 4: Mechanism of respiratory stem cell carcinogenesis.

FIG. 5: Flow chart showing triage of prospectively collected blood and corresponding lung cancer tissue from a patient with lung cancer.

FIG. 6: Imprint from an adenocarcinoma of lung illustrating multiple deletions for 3p22.1 (green signal versus centromeric 3, red signal) and 10q22-23 (gene for Surfactant A protein, green signal versus red signal for centromeric 10) throughout the lung cancer and bronchial epithelial cells on the side of the tumor consistent with a field cancerization effect.

FIG. 7: Composite diagram of lung cancer resulting from 110 resections that have been mapped for these deletions by FISH, showing that the average level of deletion in the lung cancer is 16.4% for 3p22.1 and 11.2% for 10q22-23.

FIGS. 8A-D: Patient is a 64 year-old male with “limited small cell carcinoma.” Combined Immunohistochemistry for CD45 and two-color FISH on Ficoll purified mononuclear cells from peripheral blood showing clonal abnormalities for chromosome 10 (aqua) and Surfactant A gene (red) in CD45-negative cells. FIG. 8A shows CD45-positive and -negative fluorescent cells. FIG. 8B shows the same image as FIG. 3A, note three red signals (10q22-23) and one aqua signal in several cells consistent with clonal. FIG. 8C is the merged images from A and B. FIG. 8D shows normal control with two red signals (10q22-23) and two aqua signals (centromeric 10) in CD45-positive cells. Note that non-fluorescent cells corresponding to circulating tumour cells show clonal chromosomal abnormalities, monosomy ten (aqua) and trisomy 10q22-23. Note in this specimen, a percentage of CD45-negative cells were 30% of which clonal abnormalities were detected in 64% of cells abnormality of 10q22-23 and centromeric 10 95% of cells fluorescent CD45-positive cells.

FIGS. 9A-C: Patient is a 52 year-old lady with “limited small cell carcinoma.” Combined immunohistochemistry for CD45 and two-color FISH on Ficoll purified mononuclear cells from peripheral blood showing clonal abnormalities for chromosome 3 (red) and 3p22.1 (green) in CD45-negative cells. FIG. 9A demonstrates CD45-positive and negative cells, note kidney bean shaped cell. FIG. 9B shows the same image as FIG. 9A, note three red signals (centromeric 3) and two green signals (3p22.1) consistent with clonal abnormality for deletion of 3p22.1. FIG. 9C is the merged images from 9A and 9B. FIG. 9C demonstrates the normal control with two red signals (centromeric 3) and two green signals (3p22.1) in CD45-positive cells. Note 95% of cells are fluorescent CD45-positive cells. Also note that non-fluorescent cells corresponding to circulating tumor cells show clonal chromosomal abnormalities trisomy 3 (three red signals per cell and deletion for 3p22.1 (two green signals). Note that in this specimen, there were 41.5% of CD45-negative cells, of which 62% showed clonal abnormalities.

FIG. 10: Examples of trisomy 10q22-23 and 3p22.1 in both carcinoma of lung and in peripheral blood. Top panel: Arrows indicate touch imprint non-small cell lung cancer and blood with trisomy 10q22-23. Note that these are not from the same patient, however this abnormality in lung cancer is very common and occurs in an average of 11% of cells (see FIG. 7). Bottom panel: Imprint of non-small cell carcinoma trisomy chromosome 3 (three red signals per cell) with two green signals per cell (arrow) denoting deletion of 3p22.1 This deletion has been seen to be present in the majority of NSCLC, with a mean of 20% deletion in each primary tumor. Bottom right panel shows similar pattern of deletion for 3p22.1 as that seen in the lung imprint.

FIG. 11: Patient is a 66 year-old male with bulky but limited Stage 11 small cell lung cancer. Genetic abnormalities of Epidermal Growth Factor Receptor (EGFR) red signals bottom left panel showing both over-expression or amplification (multiple red signals) compared to centromeric 7 (green signals) in imprint of adenocarcinoma of lung similar to peripheral blood (×600). Top left hand panel shows deletion of EGFR (red signals) compared to centromeric 7 (green signals), top right, peripheral blood with whole chromosome deletion of centromeric 7 and EGFR, bottom right hand panel shows polysomy of chromosome 7.

FIGS. 12A-C: FIG. 12A: Peripheral blood after Ficoll-Hypaque enrichment from a patient with squamous carcinoma, stage 1V, of lung showing 30% of mononuclear cells to be CD45 negative and 77% to be CD45 positive. FIG. 12B: Same sample stained for cytokeratin showing 20% of CTCs demonstrating faintly positive membranous staining, indicating epithelial differentiation, consistent with history of primary squamous carcinoma of lung. Note “patchy” chromatin staining in both cancer cells from Band cancer cells from cell line C. FIG. 12C: Control cell line from non-small cell carcinoma of lung showing positive cytokeratin staining.

FIG. 13: Graph demonstrates the worse survival of patients were those having cep17_Uro gaine abnormalities—Group 0 were those samples having no abnormal cells detected; Group 1 were those samples where abnormal cells were detected.

FIG. 14: Example of the slide micro-array technique taken from Li et al. (2006).

FIG. 15: Example of the slide micro-array technique taken from Li et al. (2006).

FIG. 16: ROC Curve for risk model including combined 3p, combined 10q and CTC Uro_LaV.

FIGS. 17A-B: Error bar plots comparing biomarkers across pathological stage.

FIGS. 18A-H: Error Bars Plots comparing biomarkers: Clinical versus Pathological Staging (numbers are mean values).

FIGS. 19A-E: Survival plots.

FIGS. 20A-J: Recurrence plots.

FIG. 21: Error Bar Plots Showing Percentage Deletions and Gains of EGFR(Y-axis) with the LAV probe set in PBMCs Specimens Obtained from Controls and Patients by Disease Stage (X-axis).

FIG. 22: CTCs in controls and patients with NSCLC with chromosomal abnormalities of 3p22.1/CEP3, 10q22.3/CEP10, URO and LAV probe stratified by stage. Note the trend for numbers of CTCs for all chromosomal abnormalities to increase from low stage to high stage NSCLC.

FIGS. 23A-D: Select error bar plots for cytogenetically abnormal cells (CACs) expressing different genetic abnormalities that showed a significant trend (P<0.05) increasing across stage of disease (FIG. 23A) deletions 3p22.1/CEP3 and 10q22.3/CEP10; (FIG. 23B) UroVysion 9p22.1 deletions; (FIG. 23C) UroVysion single gain; and (D) UroVysion CEP7 gain.

FIGS. 24A-H: Stage 1A adenocarcinoma, FISH: (FIGS. 24A and B) deletions 3p22.1 (green) versus CEP3 (red) CTC and tumor; (FIG. 24C) deletions 10q22.3 (green) CTC versus CEP10 (red) (FIG. 24D) polysomy 10q22.3 (green) tumor versus CEP10 (red) (FIG. 24E) gain EGFR (red) CTC; (FIG. 24F) amplification EGFR, C-myc (yellow), 5p15.2 (green), 6p11-q12 (aqua) tumor; (FIG. 24G) trisomy CEP3 (red), monosomy CEP17 (aqua) CTC; and (FIG. 24H) polysomy CEP3, CEP7 (green), CEP17, and 9p21.3 (yellow) tumor.

FIG. 25: Select Kaplan-Meier curves of progression-free survival duration with biomarkers significant at 5% level (1) Monosomy 3p22.1, P=0.024; (2) 10q22.3/CEP10 abnormalities, P=0.017; (3) EGFR deletion, P=0.034; (4) 6p deletion, P=0.010; (5) URO single gain, P=0.003; and (6) 9p21 deletion, P=0.001.

FIG. 26: Select Kaplan-Meier curves for overall survival with biomarkers significant at 10% level; (1) EGFR deletion, P=0.053; (2) 9p21 deletion, P=0.054; (3) URO single gain, P=0.015; and (4) CEP3 gain, P=0.027.

FIGS. 27A-D: FIG. 27A: CTC Comparison AZI vs AZII; FIG. 27B: Urovysion Comparison AZI vs AZII; FIG. 27C: 10q Comparison AZI vs AZII; FIG. 27D: 3p Comparison AZI vs AZII

### DETAILED DESCRIPTION OF ILLUSTRATIVE EMBODIMENTS

Circulating tumor cells (CTCs) in patients with lung cancer will show genetic abnormalities similar to that seen in the primary lung cancer. These occur in CD45-negative/diminished peripheral blood mononuclear or tumor cells in patients with lung cancer at significantly higher levels in all stages of lung cancer compared to controls. Other investigators have used immunomagnetic capture or density gradient centrifugation with immunohistochemistry and FISH to detect aneuploidy in CTCs. However all studies, while demonstrating genetic abnormalities similar to those of the primary tumor, were limited by a low cell recovery and inability to detect chromosomal abnormalities in patients with CTCs<10 per 7.5 mL blood.

Genetically abnormal mononuclear cells (or circulating tumor cells) containing the same genetic abnormality as the primary tumor are present in peripheral blood of lung cancer patients, are associated with tumor stage and tumor burden, and occur at lower levels in patients with low stage versus high stage disease. Monitoring of these cells in the peripheral blood by combined immunocytochemistry and fluorescence in situ hybridization (FISH) at both at baseline and at follow up after therapy, provide a sensitive molecular marker of response to therapy if the number of cells bearing these chromosomal or genetic abnormalities decrease. Similarly, persistence or increased numbers of cells with these deletions will indicate stable or progressive disease. For example, deletions of chromosome 3p21.3 and 3p22.1 occur simultaneously and very early on in the pathogenesis of early lung neoplasia. There are numerous tumor suppressor genes located in this portion of the genome that are highly relevant to lung cancer neoplasia (Barkan et al., 2004; Goeze et al., 2002). Similarly deletions on chromosome 10q22-23 have been frequently reported in primary lung cancer and also in metastatic lung cancer, both for small cell and non-small cell carcinoma (NSCL). Deletions of 10q22-23 furthermore are associated with an aggressive clinical course, with high levels of deletions being strongly associated with poor prognosis Jiang et al., 2005; Goeze et al., 2002; Gough et al., 2002).

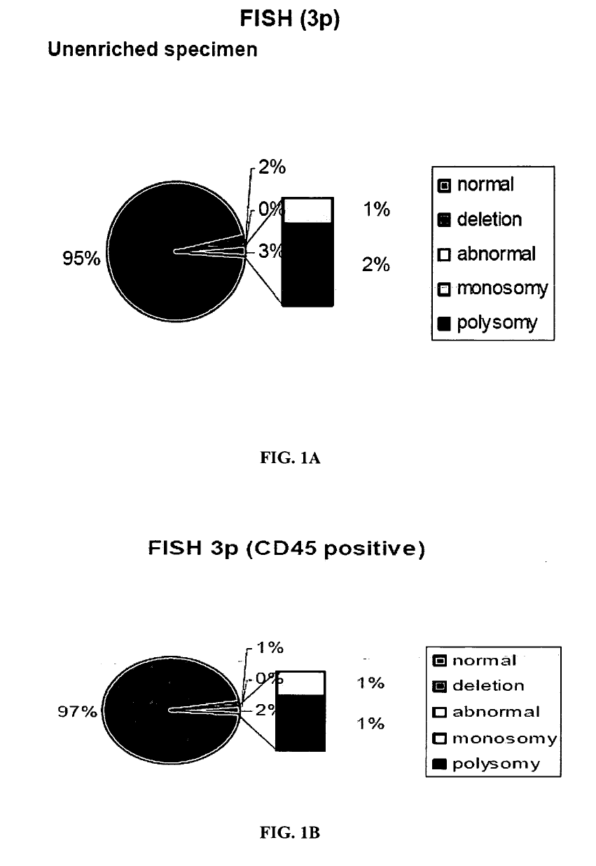

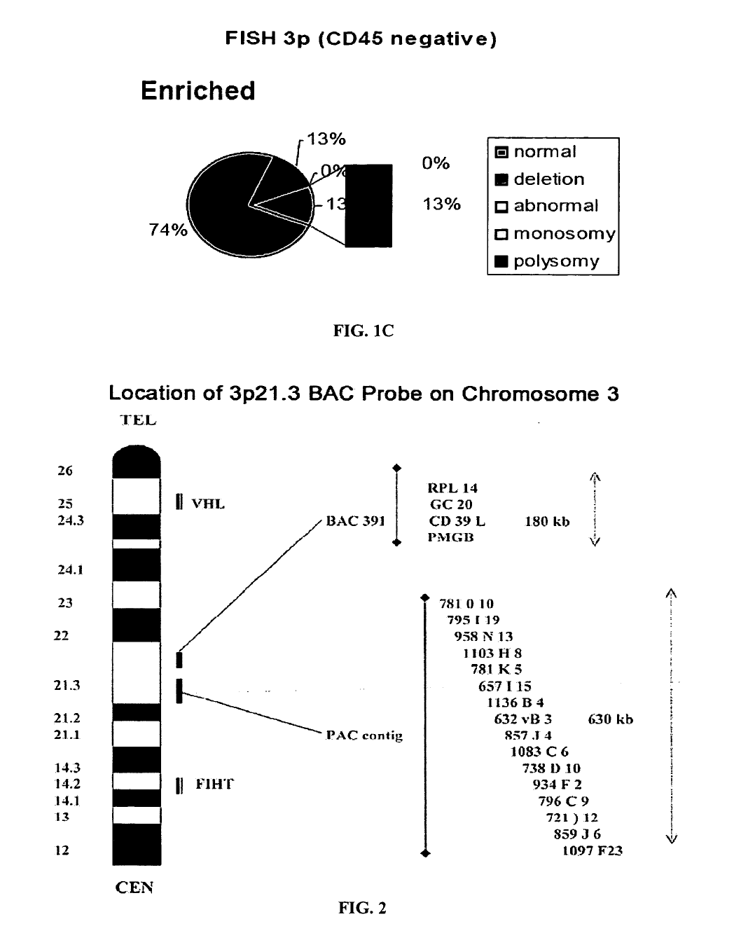

The currently disclosed approach employs a fluorescence in situ (FISH)-based assay to hybridize selected nucleic acid probes covering specific chromosomal regions or genes known to be abnormal in lung cancer to isolated mononuclear cells from the blood from subjects with lung cancer. In particular, using a gradient separation method, the tumor cells are isolated, and then sorted manually, by flow cytometry, or by image analysis into hematopoietic and non-hematopoietic cells based on CD45. The cells may be sorted based on positive or negative and diminished staining (FIGS. 1A-C). The selected cells are then subjected to multicolor FISH using a variety of different probe sets with different fluorochromes and several thousand cells are scanned and quantitated by image analysis. The scanning may be performed, for example, on an automated scanner with Fluorescence capabilities (Bioview System, Rehovoth, Israel). The results of the FISH tests in blood from subjects with cancer are analyzed compared to control subjects and compared to the FISH profile of the primary tumors of the patients. The control group includes patients who were at high risk to develop lung cancer as well as healthy subjects. The results of the CTCs prior to resection were also compared, and then these results were to imprints from the resected lung cancer using the identical set of FISH probes that were used for the CTCs.

The present invention therefore provides for methods of isolating the tumor cells from the peripheral blood, the detection of CD45-diminished and -negative non-hematopoietic cells that express abnormal FISH markers, the nucleic acid probe sets used, and methods of use, including but not limited to primary detection of cancer, follow-up after therapy and for longitudinal monitoring of disease status and response to different therapies. It has been shown that by the method of the present invention, cells with clonal genetic abnormalities could be found in peripheral blood at very high levels compared to previous methods.

This method has the benefits of 1) the ability to isolate much higher numbers of abnormal cells than had previously been described by other methods; 2) the ability to perform multicolor FISH using a variety of molecular DNA probes on a single specimen combined with immuno-fluorescence staining in order to obtain a phenotype of the CTCs and to demonstrate clonality; and 3) the ability to enrich the abnormal phenotype by “gating” only on the CD45-diminished or -negative cells.

In comparison with other methods, several orders of magnitude higher numbers of CTCs were observed than most other studies. Depending on the biomarker abnormality assayed, up to 45 CTCs per microliter were detected compared to <10 CTCs per 7.5 milliliter in most studies using immunomagnetic beads (Cristofanilli et al., 2004; Allard et al., 2004; Sieuwerts et al., 2009; Wu et al., 2009; Swennenhuis et al., 2009; Leversha et al., 2009). The percentages of CACs tended to increase significantly with disease stage, which consistently reflected tumor burden.

It should be noted that the methods described in this application are applicable for isolating circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc. In particular, it is possible for each set of tumors, to derive a set of genomic markers that are abnormal in a specific cancer subtype based on published genomic data or on genomic data generated by testing different tumors with comparative genomic hybridization (CGH) or single nucleotide polymorphisms (SNPS) and performing bioinformatics to determine over- or underexpression of different genes. Following the best choice of abnormal molecular regions to be tested, the optimal fluorescently labeled probes can be synthesized.

### I. CANCER

The present invention envisions the use of assays to detect cancer and predict its progression in conjunction with cancer therapies. In some cases, where patients are suspected to be at risk of cancer, prophylactic treatments may be employed. In other cancer subjects, diagnosis may permit early therapeutic intervention. In yet other situations, the result of the assays described herein may provide useful information regarding the need for repeated treatments, for example, where there is a likelihood of metastatic, recurrent or residual disease. Finally, the present invention may prove useful in demonstrating which therapies do and do not provide benefit to a particular patient.

Furthermore, the methods described in this application are able to be translated into a method for isolating circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc.

A. Tumorgenesis

The deletions of various genes in tumor tissue has been well studied in the art. However, there remains a need for probes that are significant for detecting early molecular events in the development of cancers, as well as molecular events that make patients susceptible to the development of cancer. Probes used for the staging of cancer are also of interest. The proposed sequence leading to tumorigenesis includes genetic instability at the cellular or submicroscopic level as demonstrated by loss or gain of chromosomes, leading to a hyperproliferative state due to theoretical acquisition of factors that confer a selective proliferative advantage. Further, at the genetic level, loss of function of cell cycle inhibitors and tumor suppressor genes (TSG), or amplification of oncogenes that drive cell proliferation, are implicated.

Following hyperplasia, a sequence of progressive degrees of dysplasia, carcinoma-in-situ and ultimately tumor invasion is recognized on histology. These histologic changes are both preceded and paralleled by a progressive accumulation of genetic damage. At the chromosomal level genetic instability is manifested by a loss or gain of chromosomes, as well as structural chromosomal changes such as translocation and inversions of chromosomes with evolution of marker chromosomes. In addition cells may undergo polyploidization. Single or multiple clones of neoplastic cells may evolve characterized in many cases by aneuploid cell populations. These can be quantitated by measuring the DNA content or ploidy relative to normal cells of the patient by techniques such as flow cytometry or image analysis.

B. Prognostic Factors and Staging

The stage of a cancer at diagnosis is an indication of how much the cancer is spread and can be one of the most important prognostic factors regarding patient survival. Staging systems are specific for each type of cancer. For example, at present the most important prognostic factor regarding the survival of patients with lung cancer of non-small cell type is the stage of disease at diagnosis. For example, the most important prognostic factor regarding the survival of patients with lung cancer of non-small cell type is the stage of disease at diagnosis. Conversely, small cell cancer usually presents with wide spread dissemination hence the staging system is less applicable. The staging system was devised based on the anatomic extent of cancer and is now know as the TNM (Tumor, Node, Metastasis) system based on anatomical size and spread within the lung and adjacent structures, regional lymph nodes and distant metastases. The only hope presently for a curative procedure lies in the operability of the tumor which can only be resected when the disease is at a low stage, that is confined to the organ of origination.

C. Grading of Tumors

The histological type and grade of lung cancers do have some prognostic impact within the stage of disease with the best prognosis being reported for stage I adenocarcinoma, with 5 year survival at 50% and 1-year survival at 65% and 59% for the bronchiolar-alveolar and papillary subtypes (Naruke et al., 1988; Travis et al., 1995; Carriaga et al., 1995). For squamous cell carcinoma and large cell carcinoma the 5 year survival is around 35%. Small cell cancer has the worst prognosis with a 5 year survival rate of only 12% for patients with localized disease (Carcy et al., 1980; Hirsh, 1983; Vallmer et al., 1985). For patients with distant metastases survival at 5 years is only 1-2% regardless of histological subtype (Naruke et al., 1988). In addition to histological subtype, it has been shown that histological grading of carcinomas within subtype is of prognostic value with well differentiated tumors having a longer overall survival than poorly differentiated neoplasms. Well differentiated localized adencarcinoma has a 69% overall survival compared to a survival rate of only 34% of patients with poorly differentiated adenocarcinoma (Hirsh, 1983). The 5 year survival rates of patients with localized squamous carcinoma have varied from 37% for well differentiated neoplasms to 25% for poorly differentiated squamous carcinomas (Ihde, 1991).

The histologic criteria for subtyping lung tumors is as follows: squamous cell carcinoma consists of a tumor with keratin formation, keratin pearl formation, and/or intercellular bridges. Adenocarcinomas consist of a tumor with definitive gland formation or mucin production in a solid tumor. Small cell carcinoma consists of a tumor composed of small cells with oval or fusiform nuclei, stippled chromatin, and indistinct nuclei. Large cell undifferentiated carcinoma consists of a tumor composed of large cells with vesicular nuclei and prominent nucleoli with no evidence of squamous or glandular differentiation. Poorly differentiated carcinoma includes tumors containing areas of both squamous and glandular differentiation.

D. Development of Carcinomas

The evolution of carcinoma of the lung is most likely representative of a field cancerization effect as a result of the entire aero-digestive system being subjected to a prolonged period of carcinogenic insults such as benzylpyrenes, asbestosis, air pollution and chemicals other carcinogenic substances in cigarette smoke or other environmental carcinogens. This concept was first proposed by Slaughter et al. (1953). Evidence for existence of a field effect is the common occurrence of multiple synchronous for metachronous second primary tumors (SPTs) that may develop throughout the aero-digestive tract in the oropharynx, upper esophagus or ipsilateral or contralateral lung.

Accompanying these molecular defects is the frequent manifestation of histologically abnormal epithelial changes including hyperplasia, metaplasia, dysplasia, and carcinoma-in-situ. It has been demonstrated in smokers that both the adjacent normal bronchial epithelium as well as the preneoplastic histological lesions may contain clones of genetically altered cells (Wistuba et al., 2000).

Licciardello et al. (1989) found a 10-40% incidence of metachronous tumors and a 9-14% incidence of synchronous SPTs in the upper and lower aero-digestive tract, mostly in patients with the earliest primary tumors SPTs may impose a higher risk than relapse from the original primary tumor and may prove to be the major threat to long term survival following successful therapy for early stage primary head, neck or lung tumors. Hence it is vitally important to follow these patients carefully for evidence of new SPTs in at risk sites for new malignancies specifically in the aero-digestive system.

In addition to chromosomal changes at the microscopic level, multiple blind bronchial biopsies may demonstrate various degrees of intraepithelial neoplasia at loci adjacent to the areas of lung cancer. Other investigators have shown that there are epithelial changes ranging from loss of cilia and basal cell hyperplasia to CIS in most light and heavy smokers and all lungs that have been surgically resected for cancer (Auerbach et al., 1961). Voravud et al. (1993) demonstrated by in-situ hybridization (ISH) studies using chromosome-specific probes for chromosomes 7 and 17 that 30-40% of histologically normal epithelium adjacent to tumor showed polysomies for these chromosomes. In addition there was a progressive increase in frequency of polysomies in the tissue closest to the carcinoma as compared to normal control oral epithelium from patients without evidence of carcinoma. The findings of genotypic abnormalities that increased closer to the area of the tumor support the concept of field cancerization. Interestingly, there was no increase in DNA content as measured in the normal appearing mucosa in a Feulgen stained section adjacent to the one where the chromosomes were measured, reflecting perhaps that insufficient DNA had been gained in order to alter the DNA index. Interestingly, a very similar increase in DNA content was noted both in dysplastic areas close to the cancer and in the cancerous areas suggesting that complex karyotypic abnormalities that are clonal have already been established in dysplastic epithelium adjacent to lung cancer. Others have also shown an increase in number of cells showing p53 mutations in dysplastic lesions closest to areas of cancer, which are invariably also p53 mutated. Other chromosomal abnormalities that have recently been demonstrated in tumors and dysplastic epithelium of smokers includes deletions of 3p, 17p, 9 p and 5q (Feder et al., 1998; Yanagisawa et al., 1996; Thiberville et al., 1995).

E. Chromosome Deletions in Lung Cancer

Small cell lung cancer (SCLC) and non-small cell lung cancer commonly display cytogenetically visible deletions on the short arm of chromosome 3 (Hirano et al., 1994; Valdivieso et al., 1994; Cheon et al., 1993; Pence et al., 1993). This 3p deletion occurs more frequently in the lung tumor tissues of patients who smoke than it does in those of nonsmoking patient. (Rice et al., 1993) Since approximately 85% lung cancer patients were heavy cigarette smokers (Mrkve et al., 1993), 3p might contain specific DNA loci related to the exposure of tobacco carcinogens. It also has been reported that 3p deletion occurs in the early stages of lung carcinogenesis, such as bronchial dysplasia (Pantel et al., 1993). In addition to cytogenetic visible deletions, loss of heterozygosity (LOH) studies have defined 3-21.3 as one of the distinct regions that undergo loss either singly or in combination (Fontanini et al., 1992; Liewald et al., 1992). Several other groups have found large homozygous deletions at 3p21.3 in lung cancer (Macchiarini et al., 1992; Miyamoto et al., 1991; Ichinose et al., 1991; Yamaoka et al., 1990). Transfer of DNA fragments from 3-21.3-3p21.2 into lung tumor cell lines could suppress the tumorigenesis (Sahin et al., 1990; Volm et al., 1989). These finding strongly suggest the presence of at least one tumor suppressor gene in this specific chromosome region whose loss will initiate lung carcinogenesis.

Cytogenetic observation of lung cancer has shown an unusual consistency in the deletion rate of chromosome 3p. In fact, small cell lung cancer (SCLC) demonstrates a 100% deletion rate within certain regions of chromosome 3p. Non small cell lung cancer (NSCLC) demonstrates a 70% deletion rate (Mitsudomi et al., 1996; Shiseki et al., 1996). Loss of heterozygosity and comparative genomic hybridization analysis have shown deletions between 3p14.2 and 3p21.3 to be the most common finding for lung carcinoma and is postulated to be the most crucial change in lung tumorigenesis (Wu et al., 1998). It has been hypothesized that band 3p21.3 is the location for lung cancer tumor suppressor genes. The hypothesis is supported by chromosome 3 transfer studies, which reduced tumorigenicity in lung adenocarcinoma.

Allelotype studies on non-small cell lung carcinoma indicated loss of genetic material on chromosome 10q in 27% of cases. Studies of chromosome 10 allelic loss have shown that there is a very high incidence of LOH in small cell lung cancer, up to 91%. (Alberola et al., 1995; Ayabe et al., 1994). A statistically significant LOH of alleles on 10q was noted in metastatic squamous cell carcinoma (SCC) in 56% of cases compared to non-metastatic SCC with LOH seen in only 14% of cases (Ayabe et al., 1994). No LOH was seen in other subtypes on NSCLC. Additionally, using micro-satellite polymorphism analysis, it was shown that a high incidence of loss exists between D10s677 and D10S1223. This region spans the long arm of chromosome 10 at bands q21-q24 and overlaps the region deleted in the a study of advanced stage high grade bladder cancers which demonstrated a high frequency of allele loss within a 2.5cM region at 10q22.3-10q23.1 (Kim et al., 1996).

### II. CD45 SELECTION

In some embodiments, the invention comprises contacting said sample with a CD45 binding agent and selecting the cells based on staining for CD45. The cells may be selected by any method known to those of skill in the art, including but not limited to standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy or a bright field examination using chromogen labeled probes such as DAB or AEC, and/or by measuring changes in gene expression using techniques well known in the art, such as PCR and gene expression profiling.

### III. GENE PROBES

The present invention comprises contacting the selected cells with a labeled nucleic acid probe, and detecting hybridized cells by fluorescence in situ hybridization. These probes may be specific for any genetic marker that is most frequently amplified or deleted in CTCs. In particular, the probes may be a 3p22.1 probe, which is a nucleic acid probe targeting RPL14, CD39L3, PMGM, or GC20, combined with centromeric 3; a 10q22-23 probe (encompassing surfactant protein A1 and A2) combined with centromeric 10; or a PI3 kinase probe. Other genetic markers may include, but are not limited to, centromeric 3, 7, 17, 9p21, 5p15.2, EGFR, C-myc8q22, and 6p22-22. For a further discussion of gene probes see U.S. Publication No. 2007/0218480, herein incorporated by reference in its entirety.

A. 3p22.1 Probe

A 3p22.1 probe is a nucleic acid probe targeting RPL14, CD39L3, PMGM, or GC20, combined with centromeric 3. The human ribosomal L14 (RPL14) gene (GenBank Accession NM_{—}003973), and the genes CD39L3 (GenBank Accession AAC39884 and AF039917), PMGM (GenBank Accession P15259 and J05073), and GC20 (GenBank Accession NM_{—}005875) were isolated from a BAC (GenBank Accession AC104186, herein incorporated by reference) and located in the 3p22.1 band within the smallest region of deletion overlap of various lung tumors (FIG. 2). The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. It is understood that the polyalanine tract plays a key role in the nonsense-mediated mRNA decay pathway that rids cells aberrant proteins and transcripts. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. See also RP11-391M1/AC104186.

Genes with a regulatory function such as the RPL14 gene, along with the genes CD39L3, PMGM, and GC20 and analogs thereof, are good candidates for diagnosis of tumorigenic events. It has been postulated that functional changes of the RPL14 protein can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

B. 10q22 Probe

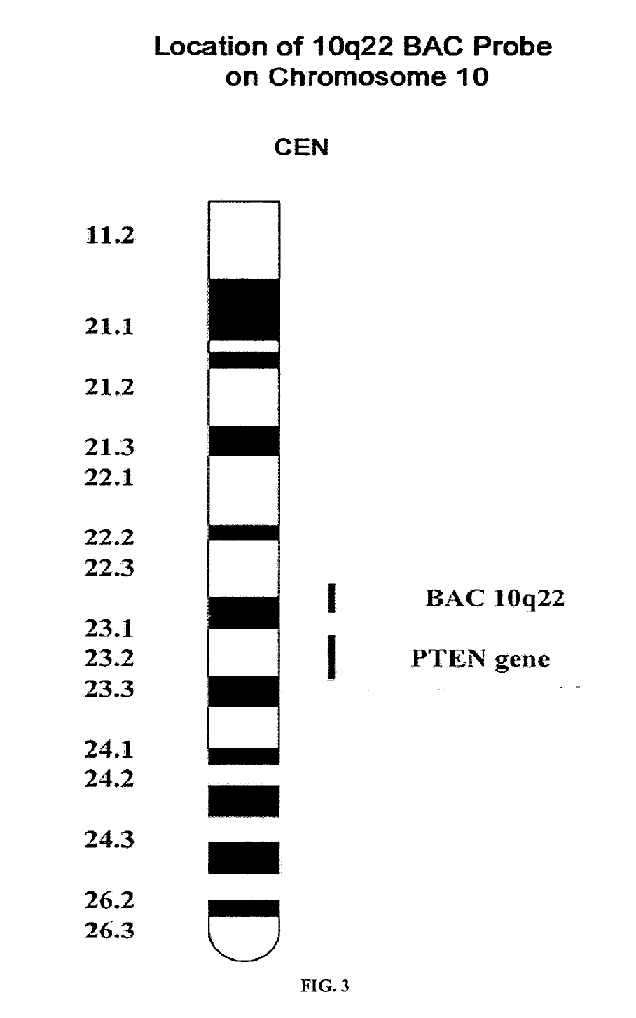

In other embodiments, the probe may be a 10q22-23 probe, which encompasses surfactant protein A1 and A2, combined with centromeric 10. The 10q22 BAC (46b12) is 200 Kb and is adjacent and centromeric to PTEN/MMAC1 (GenBank Accession AF067844), which is at 10q22-23 and can be purchased through Research Genetics (Huntsville, Ala.) (FIG. 3). Alterations to 10q22-25 has been associated with multiple tumors, including lung, prostate, renal, and endomentrial carcinomas, melanoma, and meningiomas, suggesting the possible suppressive locus affecting several cancers in this region. The PTEN/MMAC1 gene, encoding a dual-specificity phosphatase, is located in this region, and has been isolated as a tumor suppressor gene that is altered in several types of human tumors including brain, bladder, breast and prostate cancers. PTEN/MMAC1 mutations have been found in some cancer cell lines, xenografts, and hormone refractory cancer tissue specimens. Because the inventor's 10q22 BAC DNA sequence is adjacent to this region, the DNA sequences in the BAC 10q22 may be involved in the genesis and/or progression of human lung cancer. See also RP11-506M13/AC068139.6

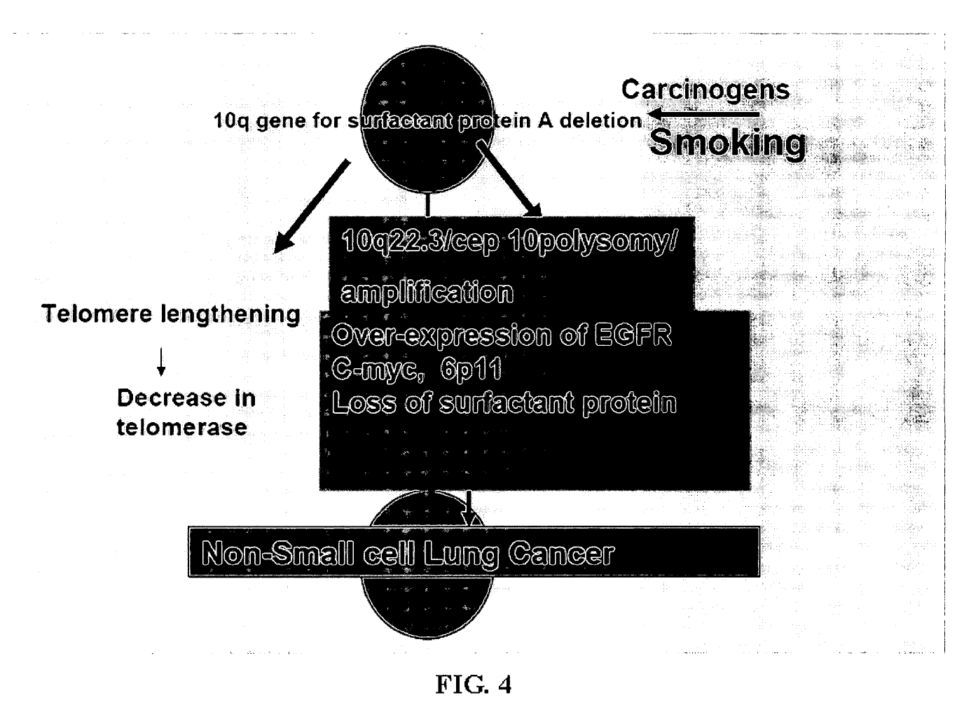

Pulmonary-associated surfactant protein A1(SP-A) is located at 10q22.3. Surfactant protein-A-phospholipid-protein complex lowers the surface tension in the alveoli of the lung and plays a major role in host defense in the lung. Surfactant protein-A1 is also present in alveolar type-2 cells, which are believed to be putative stem cells of the lung. It is known that type-2 cells participate in repair and regeneration after alveolar damage. Thus, it is possible that the type-2 cells express telomerase and C-MYC, which leads to the loss of the surfactant protein and the development of non-small cell lung cancer (FIG. 4). The 10q22 probe is useful in the further development of clinical biomarkers for the early detection of neoplastic events, for risk assessment and monitoring the efficacy of chemoprevention therapy.

C. PI3 kinase

Because of the high correlation between cancers and circulating cells, any other biomarker such as PI3 kinase could be used to monitor response to therapy if a PI3 kinase inhibitor were used.

D. Commercial Probe Sets

Any commercial probes or probe sets may also be used with the present invention. For example, the UroVysion DNA probe set (Vysis/Abbott Molecular, Des Plaines, Ill.) may be used, which includes probes directed to centromeric 3, centromeric 7, centromeric 17, 9p21.3. It has been established that UroVysion probes detect early changes of lung cancer. In other embodiments, the LaVysion DNA probe set (Vysis/Abbott Molecular, Des Plaines, Ill.), which includes probes to 7p12 (epidermal growth factor receptor); 8q24.12-q24.13 (MYC); 6p11.1-q11 (chromosome enumeration (Probe CEP 6); and 5p15.2 (encompassing the SEMA5A gene), may be used. It has been noted that the LaVysion probe set detects higher stages or more advanced stags of lung cancer. Furthermore, a single probe set directed to centromeric7/7p12 (epidermal growth factor receptor) may also be used with the present invention.

### IV. METHODS FOR ASSESSING GENE STRUCTURE

In accordance with the present invention, one will utilize various probes to examine the structure of genomic DNA from patient samples. A wide variety of methods may be employed to detect changes in the structure of various chromosomal regions. The following is a non-limiting discussion of such methods.

A. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout it's length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.) Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for loss of allelic heterozygosity is that the (a) FISH is easily and rapidly performed on cells of interest and can be used on paraffin-embedded, or fresh or frozen tissue allowing the use of micro-dissection (b) specific gene changes can be analyzed on a cell by cell basis in relationship to centomeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted) (c) PCR cannot identify amplification of genes d) FISH using bacterial artificial chromosomes (BACs) permits easy detection and localization on specific chromosomes of genes of interest which have been isolated using specific primer pairs.

Chromogenic in situ hybridzation (CISH) enables the gain genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference.

B. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present invention (Wu et al., 1989, incorporated herein by reference in its entirety).

C. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

D. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

E. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present invention provides methods by which any or all of these types of analyses may be used.

F. Kit Components

All the essential materials and reagents required for detecting changes in the chromosomal regions discussed above may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification, and optionally labeling agents such as those used in FISH. Such kits also generally will comprise, in suitable means, distinct containers for each individual reagent and enzyme as well as for each primer or probe.

G. Chip Technologies

Specifically contemplated by the present inventors are chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules using methods such as fluorescence, conductance, mass spectrometry, radiolabeling, optical scanning, or electrophoresis. See also Pease et al. (1994); Fodor et al. (1991).

Biologically active DNA probes may be directly or indirectly immobilized onto a surface to ensure optimal contact and maximum detection. When immobilized onto a substrate, the gene probes are stabilized and therefore may be used repetitively. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Immobilization of the gene probes may be achieved by a variety of methods involving either non-covalent or covalent interactions between the immobilized DNA comprising an anchorable moiety and an anchor. DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. Gene probe may be bound directly to membranes using ultraviolet radiation. With nitrocellous membranes, the probes are spotted onto the membranes. A UV light source is used to irradiate the spots and induce cross-linking. An alternative method for cross-linking involves baking the spotted membranes at 80° C. for two hours in vacuum.

Immobilization can consist of the non-covalent coating of a solid phase with streptavidin or avidin and the subsequent immobilization of a biotinylated polynucleotide (Holmstrom, 1993). Precoating a polystyrene or glass solid phase with poly-L-Lys or poly L-Lys, Phe, followed by the covalent attachment of either amino- or sulthydryl-modified polynucleotides using bifunctional crosslinking reagents (Running, 1990 and Newton, 1993) can also be used to immobilize the probe onto a surface.

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287) describes a method of non-covalently immobilizing nucleic acid molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the synthetic nucleic acid and the cationic detergent or salt. The support containing the immobilized nucleic acid may be washed with an aqueous solution containing a non-ionic detergent without removing the attached molecules.

There are two common variants of chip-based DNA technologies involving DNA microarrays with known sequence identity. For one, a probe cDNA (5005,000 bases long) is immobilized to a solid surface such as glass using robot spotting and exposed to a set of targets either separately or in a mixture. This method, “traditionally” called DNA microarray, is widely considered as developed at Stanford University. A recent article by Ekins and Chu (1999) provides some relevant details. The other variant includes an array of oligonucleotide (20˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its products under the GeneChip® trademark.

### V. NUCLEIC ACIDS

The inventors provides a method comprises a step of contacting the selected cells with a labeled nucleic acid probe forming hybridized cells, wherein hybridization of the labeled nucleic acid is indicative of a CTC. However, the present invention is not limited to the use of the specific nucleic acid segments disclosed herein. Rather, a variety of alternative probes that target the same regions/polymorphisms may be employed.

A. Probes and Primers

Naturally, the present invention encompasses DNA segments that are complementary, or essentially complementary, to target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a target nucleic acid segment under relatively stringent conditions such as those described herein. These probes may span hundreds or thousands of base pairs.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of about 8, 9, 10, 11, 12, 13, 14, 15, 16, 17, 18, 19, 20, 25, 30, 35, 40, 45, 50, 55, 60, 65, 70, 75, 80, 85, 90, 95, 100, 250, 500, 700, 722, 900, 992, 1000, 1500, 2000, 2500, 2800, 3000, 3500, 3800, 4000, 5000 or more base pairs will be used, although others are contemplated. As mentioned above, longer polynucleotides encoding 10,000, 50,000, 100,000, 150,00, 200,000, 250,000, 300,000 and 500,000 bases are contemplated. Such oligonucleotides and polynucleotides will find use, for example, as probes in FISH, Southern and Northern blots and as primers in amplification reactions.

It will be understood that this invention is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present invention are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

B. Labeling of Probes

In certain embodiments, it will be advantageous to employ nucleic acid sequences of the present invention in combination with an appropriate means, such as a label, for determining hybridization. A wide variety of appropriate indicator means are known in the art, including fluorescent, radioactive, chemiluminescent, electroluminescent, enzymatic tag or other ligands, such as avidin/biotin, antibodies, affinity labels, etc., which are capable of being detected. In preferred embodiments, one may desire to employ a fluorescent label such as digoxigenin, spectrum orange, fluorosein, eosin, an acridine dye, a rhodamine, Alexa 350, Alexa 430, AMCA, BODIPY 630/650, BODIPY 650/665, BODIPY-FL, BODIPY-R6G, BODIPY-TMR, BODIPY-TRX, cascade blue, Cyt, Cy3, Cy5,6-FAM, HEX, 6-JOE, Oregon green 488, Oregon green 500, Oregon green 514, pacific blue, REG, ROX, TAMRA, TET, or Texas red.

In the case of enzyme tags such as urease alkaline phosphatase or peroxidase, colorimetric indicator substrates are known which can be employed to provide a detection means visible to the human eye or spectrophotometrically, to identify specific hybridization with complementary nucleic acid-containing samples. Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

The indicator means may be attached directly to the probe, or it may be attached through antigen bonding. In preferred embodiments, digoxigenin is attached to the probe before denaturization and a fluorophore labeled anti-digoxigenin FAB fragment is added after hybridization.

C. Hybridization Conditions

Suitable hybridization conditions will be well known to those of skill in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

### VI. BIOMARKERS AND OTHER RISK FACTORS

Various biomarkers of prognostic significance can be used in conjunction with the specific nucleic acid probes discussed above. These biomarkers could aid in predicting the survival in low stage cancers and the progression from preneoplastic lesions to invasive lung cancer. These markers can include proliferation activity as measured by Ki-67 (MIB1), angiogenesis as quantitated by expression of VEGF and microvessels using CD34, oncogene expression as measured by erb B2, and loss of tumor suppresser genes as measured by p53 expression.

Multiple biomarker candidates have been implicated in the evolution of neoplastic lung lesions. Bio-markers that have been studies include general genomic markers including chromosomal alterations, specific genomic markers such as alterations in proto-oncogenes such as K-Ras, Erbβ1/EGFR, Cyclin D; proliferation markers such as Ki67 or PCNA, squamous differentiation markers, and nuclear retinoid receptors (Papadimitrakopoulou et al., 1996) The latter are particularly interesting as they may be modulated by specific chemopreventive drugs such as 13-cis-retinoic acid or 4HPR and culminate in apoptosis of the defective cells with restoration of a normally differentiated mucosa (Zou et al., 1998).

A. Tumor Angiogenesis by Microvessel Counts

Tumor angiogenesis can be quantitated by microvessel density and is a viable prognostic factor in stage 1 NSCLC. Tumor microvessel density appears to be a good predictor of survival in stage 1 NSCLC.

B. Vascular Endothelial Growth Factor (VEGF)

VEGF (3, 6-8 ch 4) an endothelial cell specific mitogen is an important regulator of tumor angiogenesis who's expression correlates well with lymph node metastases and is a good indirect indicator of tumor agniogenesis. VEGF in turn is upregulated by P53 protein accumulation in NSCLC.

C. p53

The role of p53 mutations in predicting progression and survival of patients with NSCLC is widely debated. Although few studies imply a negligible role, the majority of the studies provide compelling evidence regarding the role of p53 as one of the prognostic factors in NSCLC. The important role of p53 in the biology of NSCLC has been the basis for adenovirus mediated p53 gene transfer in patients with advanced NSCLC (Carcy et al., 1980). In addition p53 has also been shown to be an independent predictor of chemotherapy response in NSCLC. In a recent study (Vallmer et al., 1985), the importance of p53 accumulation in preinvasive bronchial lesions from patients with lung cancer and those who did not progress to cancer were studied. It was demonstrated that p53 accumulation in preneoplastic lesions had a higher rate of progression to invasion than did p53 negative lesions.

D. c-erb-B2

Similar to p53, c-erg-B2 (Her2/neu) expression has also been shown to be a good marker of metastatic propensity and an indicator of survival in these tumors.

E. Ki-67 Proliferation Marker

In addition to the above markers, tumor proliferation index as measured by the extent of labeling of tumor cells for Ki-67, a nuclear antigen expressed throughout cell cycle correlates significantly with clinical outcome in Stage 1 NSCLC (Feinstein et al., 1970). The higher the tumor proliferation index the poorer is the disease free survival labeling indices provides significant complementary, if not independent prognostic information in Stage 1 NSCLC, and helps in the identification of a subset of patients with Stage 1 NSCLC who may need more aggressive therapy.

Alterations in the 3p21.3 and 10q22 loci are known to be associated with a number of cancers. More specifically, point mutations, deletions, insertions or regulatory perturbations relating to the 3p21.3 and 10q22 loci may cause cancer or promote cancer development, cause or promoter tumor progression at a primary site, and/or cause or promote metastasis. Other phenomena at the 3p21.3 and 10q22 loci include angiogenesis and tissue invasion. Thus, the present inventors have demonstrated that deletions at 3p21.3 and 10q22 can be used not only as a diagnostic or prognostic indicator of cancer, but to predict specific events in cancer development, progression and therapy.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited.

F. Surfactant Protein A

There are four main surfactant proteins: SP-A, B, C, and D. SP-A and D are hydrophilic, while SP-B and C are hydrophobic. The proteins are very sensitive to experimental conditions (temperature, pH, concentration, substances such as calcium, and so on). Moreover, their effects tend to overlap and thus it is difficult to pinpoint the specific role of each protein.

SP-A was the first surfactant protein to be identified, and is also the most abundant (Ingenito et al., 1999). Its molecular mass varies from 26-38 kDa. (Perez-Gil et al., 1998). The protein has a “bouquet” structure of six trimers (Haagsman and Diemel, 2001), and can be found in an open or closed form depending on the other substances present in the system. Calcium ions produce the closed-bouquet form. (Palaniyar et al., 1998).

SP-A plays a role in immune defense. It is also involved in surfactant transport/adsorption (with other proteins). SP-A is necessary for the production of tubular myelin, a lipid transport structure unique to the lungs. Tubular myelin consists of square tubes of lipid lined with protein (Palaniyar et al., 2001). Mice genetically engineered to lack SP-A have normal lung structure and surfactant function, and it is possible that SP-A's beneficial surfactant properties are only evident under situations of stress (Korfhagen et al., 1996).

G. Patient Interview and Other Risk Factors

In addition to analyzing the presence or absence of polymorphisms, as discussed above, it my be desirable to evaluate additional factors in a patient. For example, a patient interview, which would include a smoking history (years smoking, pack/day, etc.) is highly relevant to the diagnosis/prognosis. Also, the presence or absence of morphologic changes in sputum cells (squamous metaplasia, dysplasia, etc.) and a genetic instability score (genetic instability=composing the sum of abnormalities from various combinations in epithelial and neutrophils in sputum and/or peripheral blood cells or bone marrow cells or stem cells isolated from blood or bone marrow) may be used.

### VII. SAMPLES

In accordance with the present invention, one will obtain a biological sample that contains blood cells. Various embodiments include paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, colon, skin, stomach, spleen, lymph node, bone marrow or kidney. Other embodiments include fluid samples such as blood samples.

In some embodiments of the invention, a biological sample is obtained from a patient. The biological sample will contain blood cells from the patient. Typically, the sample is isolated from a biological sample taken from the individual, such as a blood sample or tissue sample using standard techniques such as disclosed in Jones (1963) which is hereby incorporated by reference. Collection of the samples may be by any suitable method, although in some aspects collection is by needle, catheter, syringe, scrapings, and so forth.

The sample may be prepared in any manner known to those of skill in the art. For example, the circulating epithelial cells from peripheral blood may be isolated from buffy layer following Ficoll-Hypaque gradient separation, allowing for enrichment of mononuclear cells (lymphocytes and epithelial cells). Other methods known to those of skill in the art may also be used to prepare the sample.

Nucleic acids are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. Depending on the format, the specific nucleic acid of interest is identified in the sample directly using amplification or with a second, known nucleic acid following amplification.

Following detection, one may compare the results seen in a given sample with a statistically significant reference group of samples from normal patients and patients that have or lack alterations in the various chromosome loci and control regions. In this way, one then correlates the amount or kind of alterations detected with various clinical states and treatment options.

### VIII. CANCER TREATMENTS

In some embodiments, the invention provides compositions and methods for the diagnosis and treatment of breast cancer. In one embodiment, the invention provides a method of determining the treatment of cancer based on whether the level of CTCs is high in comparison to a control. The treatment may be a conventional cancer treatment. One of skill in the art will be aware of many treatments that may be combined with the methods of the present invention, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

B. Treatments

In some embodiments, the method further comprises treating a patient with breast cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis, a novel targeted therapy such as a tyrosine kinase inhibitor, or an anti-VEGF antibody, or other therapeutic intervention. It also is conceivable that more than one administration of the treatment will be desired.

### 1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gene silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Gene Therapy

In yet another embodiment, the secondary treatment is a gene therapy in which a therapeutic polynucleotide is administered before, after, or at the same time as a H2A.Z targeting agent is administered. Delivery of a H2A.Z targeting agent in conjunction with a vector encoding one of the following gene products may have a combined anti-hyperproliferative effect on target tissues. A variety of proteins are encompassed within the invention, some of which are described below.

### a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

### b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/H2A.Z, DBCCR-1, FCC, rsk-3, p27, p27/p16 fusions, p21/p27 fusions, anti-thrombotic genes (e.g., COX-1, TFPI), PGS, Dp, E2F, vas, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

### c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

### d. RNA Interference (RNAi)

In certain embodiments, the H2A.Z inhibitor is a double-stranded RNA (dsRNA) directed to an mRNA for H2A.Z.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

### e. siRNA

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

### f. Production of Inhibitory Nucleic Acids

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ˜21 nt siRNA-like molecules (Brummelkamp et al., 2002). The siRNA-like molecules can, in turn, bring about gene-specific silencing in the transfected mammalian cells.

### g. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### 5. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### IX. EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Chromosomal Abnormalities

Lung cancer specimens and sputa evaluated with genome specific probes for lung cancer demonstrated that the unique DNA probes, 3p22.1 and 10q22-23 are both early markers of neoplasia and are associated with poor prognosis. Abnormalities of these biomarkers are present in cancer cells, and morphologically benign epithelial cells in the cancer field, as well as neutrophils and macrophages from sputum. Genetic abnormalities involving 3p22.1 and 10q22-23 also occur in CD45-negative peripheral blood mononuclear cells or circulating tumor cells (CTCs) in patients with lung cancer, who have significantly higher genetic abnormalities in these cells, compared to control bloods from high risk patients.

### Methods

### Methods of Testing Specimens for Chromosomal Abnormalities

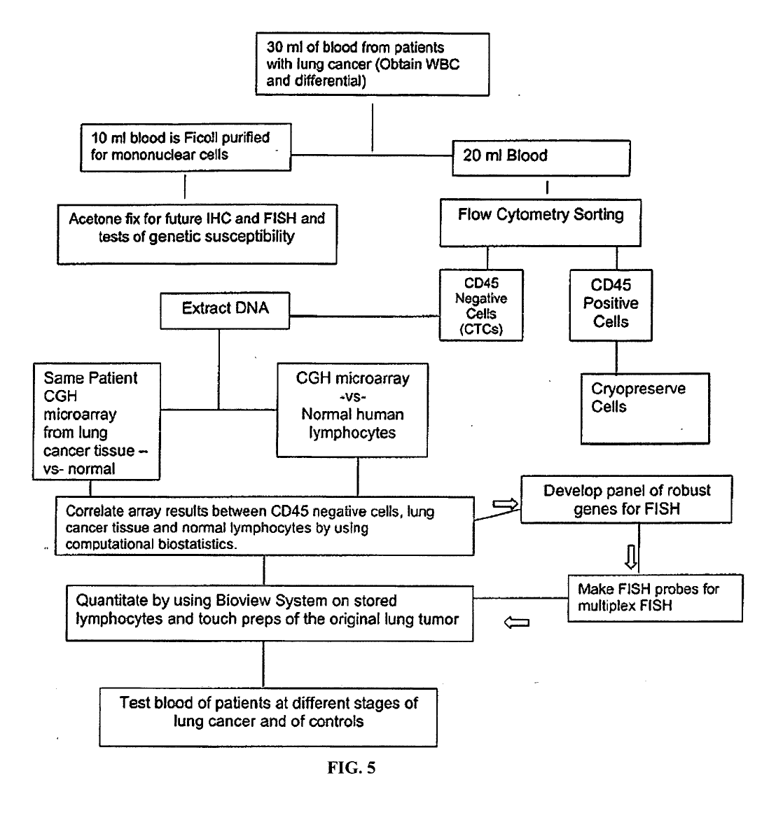

The specimens were tested as follows (see FIG. 5). First, the circulating epithelial cells from 30 ml peripheral blood of 50 patients with established lung carcinoma were isolated from buffy layer following Ficoll-Hypaque gradient separation allowing for enrichment of mononuclear cells (lymphocytes and epithelial cells). Then, mononuclear cells were counted on a Coulter counter and stained with an antibody to CD45. For 20 patients (see statistical considerations below for reasoning behind choice of number of 20 specimens). CD45-negative cells were subjected to a flow cytometry sort to produce a specimen composed predominantly of CD45-negative cells or CTCs. The CD45-positive cells may alternatively or additionally be selected. From this specimen of CD45-negative cells, DNA are isolated and subjected to the Agilent CGH microarray platform of 40,000 genes paired together with normal pooled human lymphocytes. Twenty corresponding lung tumors derived from the same patients and also paired with normal pooled human lymphocytes are similarly tested.

Based on the results of the lung cancer and CTC micro-array, a panel of lung cancer specific genetic markers that accurately predicts for metastases or CTCs and poor clinical outcome is derived. A limited set of the most promising FISH probes based on the DNA sequences from the microarray gene-data banks will be constructed. From 50 lung cancer patients of different clinical stages and fifty controls (25 smokers, 25 non-smokers) up to three hundred mononuclear cells concentrated by Ficoll gradient were subjected to immunocytochemistry for CD45 labeled with F1TC followed by two-color or four color FISH assays using a combination of centromeric probes and locus specific probes developed in house or commercially purchased.

The cells were analyzed using a high-through put fluorescent image analyzer (Bioview Duet, Rehovoth, Israel) with an existing custom-made software program specific for these probe sets. First all the CD45-positive and -negative cells are canned and their “address” recorded. Slides are then stained for FISH and again scanned on the Bioview instrument. The final display shows two side-by-side images of the same cell: the initial CD45-positive (cell membrane fluorescent) or -negative (cell membrane negative for fluorescence) cell and the same cell with the nuclear fluorescent signals. Up to four fluorescent signals, each representing a different genetic locus may be imaged in hundreds of cells and the results recorded on a per cell basis. Overall results are expressed as a pie-chart showing percentage of total chromosomal abnormalities (percentage deletions, or polysomies or aneusomies) for each genetic locus tested in CD45-negative cells. Before accepting these results, each case is quality controlled by an technologist who may accept or reject each cell scored, depending on degree of cell preservation, good quality FISH signals and appropriate negative and positive controls.

### Flow Cytometry

A FACSVantage SE Turbo Sorting Flow Cytometer (Becton Dickinson) can analyze and sort fluorochrome-labeled cells using three-beam excitation, including UV. Sorting delivers fluorochrome-labeled cells at a high purity from rare subpopulations and at a high speed (up to 25,000 cells/second) The FACSVantage SE Turbo Sorting Flow Cytometer is used to isolate CD45-negative cells at a high purity from mononuclear blood cells obtained from cancer patients The highly enriched CD45-negative cells can then be analyzed by fluorescence in-situ hybridization (FISH) for the presence/absence of a specific molecular phenotype.

### Immunofluoresence Staining for CD45

Antigen retrieval was done by incubating the slide (cytospin prepared from peripheral blood processed by Ficoll-Hypaque technique and fixed in acetone) for 10 minutes in citrate buffer in the steamer Blocking serum (bovine serum albumin) was applied to the slides for 30 minutes at room temperature, and then slides were incubated with mouse monoclonal antibody against CD45 (leukocyte common antigen) clone PD7/2 and 281 1 (Dako Corporation, Carpenteria, Calif.) at a dilution of 1:40 for 1 hour Slides were washed in 1×PBS for 5 minutes and F1TC dye conjugated Affinity Pure Donkey Anti-Mouse IgG (Jackson Immuno Research Laboratories, INC, West Grove, Pa.) at a dilution of 1:200 was applied for 1 hour, washed in 1×PBS for 5 minutes Slides were then counterstained with 10 I of 14 g/ml 4,6-diaminidino-2-phenylidole (DAPI) in Vectashield antifade solution (Vector Laboratories) and coverslipped. Slides were imaged at magnification 63× for 10 fields and x and y coordinates were noted.

### Fluorescence In Situ Hybridization (FISH)

CD45 slide was washed in 1×PBS for 5 minutes and fixed in FISH fixative (methanoVacetic acid in a 3:1 ratio) for 30 minutes. Slide was then pretreated with 2× sodium saline citrate (SSC) for 2 minutes at 74° C. and digested with 0.5 g/ml Protease (Vysis Inc, Downers Grove, Ill.) in 0.02N HCL, pH 2.0 at 37° C. for 10 minutes, washed with water, rinsed in I×PBS for 5 minutes, fixed in 1% Formaldehyde for 5 minutes and again rinsed in I×PBS, finally dehydrated through series of graded alcohol and air-dried The 2-color probe mixture for chromosomes centromeric 3 (Vysis Inc., Downers Grove, Ill.), 3p22 1, and chromosome centromeric 10 (Vysis), 10q23 (home brewed) was applied to the slides, coverslipped, sealed with rubber cement, and co-denatured on HYBRITE machine at 74° C. for 5 minutes and incubated in a humid chamber overnight at 37° C. for hybridization. After hybridization for 16 hours, slides were washed in 0 4×SSC/O 3% Nonidet P-40 for 2 minutes at 74OC, transferred to 2×SSC/O 1% Nonidet P-40 at room temperature for 1 minute, and drained. Slides were then counterstained and mounted with 10 I of 14 g/ml 4,6-diaminidino-2-phenylidole (DAPI) in Vectashield antifade solution (Vector Laboratories) and coverslipped.

The slides were scanned under a fluorescent microscope (Leica DMLB) equipped with an epi-illumination system, 100 watt mercury lamp, and Vysis filter set DAPI single band pass (DAPI counterstain), Spectrum Red/Green dual band pass, Spectrum Green single band pass, Aqua single band pass and yellow single band pass at 100× magnification Fields were matched to corresponding CD45 immunofluorescent images by x and y coordinates and imaged for DAPI, red, green, aqua and gold signals for different chromosomes. One hundred nonoverlapping cells and nuclei with distinct signals were counted, for chromosomes 3, 10, 3p21 and 10q23.

### Immunofluorescence Staining for Cytokeratin

Coverslip was removed and washed in 1×PBS for 5 minutes. Blocking serum (bovine serum albumin) was applied to the slide for 30 minutes at room temperature, and slide was then incubated with primary wide spectrum cytokeratin of polyclonal rabbit anti-human antibody (Abcam), for 1 hour at room temperature. Slide was washed in 1×PBS for 5 minutes and Texas Red dye conjugated Affinity Pure Donkey Anti-Mouse IgG (Jackson Immuno Research Laboratories, INC, West Grove, Pa.) at a dilution of 1:200 was applied for 1 hour, washed in 1×PBS for 5 minutes. Slide was then counterstained with 10 I of 14 g/ml 4,6-diaminidino-2-phenylidole (DAPI) in Vectashield antifade solution (Vector Laboratories) and coverslipped. Fields were matched with previous fluorescent CD45 and 4 color FISH images and were again imaged for fluorescent cytokeratin staining

### DNA Extraction

To isolate genomic DNA, CD45-negative sorted lymphocytes (1×10⁸ lymphocytes), are treated with cell lysis solution. Cell nuclei and mitochondria are pelleted by centrifugation The pellet is resuspended in protease solution to denature the protein, excess protease digests the denatured proteins into smaller fragments and strip the genomic DNA of all bound proteins, facilitating efficient removal during purification DNA is precipitated by addition of isopropanol, recovered by centrifugation, washed in 70% ethanol, dried, and resuspended in hydration buffer (10 mM Tris CI, pH 8 5). DNA yield is determined from the concentration of DNA in eluate, measured by absorbance at 260 nm and 280 nm. Purity is determined by calculating the ratio of absorbance at 260 nm to absorbance at 280 nm Pure DNA has an A260/A280 ratio of 1.7-1.9. The precise length of genomic DNA is determined by pulsed-field gel electrophoresis (PFGE) through an agrose gel.

### The Agilent Human Genome CGH Microarray

The Agilent Human Genome CGH Microarray (G2519A) provides genome-wide coverage with an emphasis on the most commonly studied genomic coding regions and cancer-related genes. It includes 40,000 probes that span the human genome with an average spatial resolution of approximately 75 kb, including coding and noncoding sequences It includes one probe per gene for RefSeq and Genbank Known Genes and three probes for each of approximately 1,100 known cancer genes of importance. The remaining probes are distributed to cover the rest of the genome, with an emphasis on less well known and predicted gene sequences from public databases. Designed specifically for CGH experiments, this microarray delivers CGH performance superior to microarrays designed for gene expression. Using 60-mer oligonucleotide probes, the microarray provides very high sensitivity; enabling researchers to reliably identify both highly localized and broadly extended single copy deletions, homozygous gene deletions and amplicons

The gene-focused content of the Agilent CGH array facilitates comparison of CGH and gene expression data so that researchers can correlate genomic copy number changes with gene expression changes. Agilent's array-CGH solution requires only 25 nanograms of total genomic DNA to detect chromosomal changes across the entire genome. By comparison, scientists using other oligo microarrays have typically needed to use 10 times more DNA and significantly reduce the complexity of their genomic samples, usually by amplifying only specific DNA regions. The use of total genomic DNA improves experimental design and ease of use

### Tests of Genetic Susceptibility

The CBMN test was performed using the cytochalasin B technique described by Fenech and Morley and following recommendations from The International Collaborative Project on Micronucleus Frequency in Human Populations (HUMN Project) (22) to measure MN, NPBs and NBUDs in untreated cells and NNK-treated cells. Duplicate lymphocyte cultures were prepared for each study subject. Each culture contained 2.0×10⁶ cells in 5 mL RPM1 1640 medium supplemented with 100 U/mL penicillin, 100 μg/mL streptomycin, 10% fetal bovine serum, and 2 mM L-glutamine (Gibco-Invitrogen, Carlsbad, Calif.) and 1% phytohemagglutinin (Remel, Lenexa, Kans.). For the cultures treated with NNK, 24 hours after initiation, the PBLs were centrifuged and the supernatant growth medium was removed and reserved. The PBLs were resuspended in 5 mL of serum-free RPM1 1640 medium supplemented with 0.24 mM NNK (CAS No 64091-91-4, National Cancer Institute, Midwest Carcinogen Repository, Kansas City, Mo.) and incubated at 37° C. in the presence of 5% COz for 2 hours. Next, the PBLs were washed twice with serum-free RPM1 1640, transferred to clean tubes and re-incubated for 48 hours in the reserved supernatant At 44 hours after initiation, cells were blocked in cytokinesis by adding cytochalasin B (Sigma, St Louis, Mo.; final concentration 4 μg/mL). Similarly, cultures for the determination of spontaneous damage (untreated cells) were handled in the same manner, with the exception of treatment with NNK. The total incubation time for all cultures was 72 hours. After incubation, the cells were fixed in 3:1 methanol:glacial acetic acid, dropped onto clean microscopic slides, air-dried and stained with Giemsa stain. For each sample, 1000 binucleated cells were scored blindly using a Nikon E-400 light optical microscope following the scoring criteria outlined by HUMN Project (2,11,23); the numbers of MN, NPBs, and NBUDs per 1000 binucleated cells were recorded For quality control, 20% of the slides were randomly selected and blindly rescored and the results compared with the original scoring

### Statistical Considerations

Confirming correct identification of CTCs

For an individual mutation, the probability of detecting it with array-based comparative genomic hybridization (aCGH) depends on its length; longer abnormalities are more likely to be detected successfully. If an abnormality is truly present in both the primary tumor and the CTCs, and if the probability of detecting that abnormality on a single array is p, then the probability of detecting it in both places is p² (Table 1). Fortunately, the same phenomenon affects the false positive rate. Assuming that false detections occur independently in the primary tumor and the CTCs, the chance of falsely detecting the same abnormality also goes down as the square of the probability. If the inventors allow for a false positive rate of 1%, the inventors will only say that an abnormality falsely occurs in both sites in 0.01% of each patient's loci. Since the Agilent aCGH platform includes 44,000 probes, using this cutoff is expected to produce around 4.4 false positive loci.

TABLE 1
Probability of detecting an abnormality jointly in tumor and CTCs as a
function of the probability of detecting it in one site.

| | | | | | | | | |
|---|---|---|---|---|---|---|---|---|
| Single | 0.99 | 0.95 | 0.90 | 0.80 | 0.70 | 0.60 | 0.50 | 0.01 |
| Detection | | | | | | | | |
| Joint | 0.98 | 0.90 | 0.81 | 0.64 | 0.49 | 0.36 | 0.25 | 0.0001 |
| Detection | | | | | | | | |

Selecting Markers that are Consistently Present in CTCs and Primary Tumors

For this analysis, the inventors looked at loci that had been detected by aCGH as amplified or deleted in both the primary tumor and CTCs of the same lung cancer patient. If the inventors sample N patients, then the number X of times the inventors see a particular abnormality (marker) will be a binomial random variable, where the probability of success is the product of the penetrance of the marker and of the (joint) detection probability described aboe (Table 2). As noted above, the probability of seeing the same marker by chance in both the primary tumor and the CTCs of an individual is 0.0001. The probability of seeing this same abnormality incorrectly in 2 different individuals is less than 10⁻⁶, when N is between 10 and 1000. So, as long as the inventors set a cutoff greater than 2 for a repeated abnormality, the inventors have adequate statistical significance.

TABLE 2
Probability of successfully detecting an abnormality
in tumor and CTC in an individual

| Detection Probability | Penetrance = 0.30 | 0.60 | 0.90 |
|---|---|---|---|
| 0.50 | 0.15 | 0.30 | 0.45 |
| 0.60 | 0.18 | 0.36 | 0.54 |
| 0.70 | 0.21 | 0.42 | 0.63 |
| 0.80 | 0.24 | 0.48 | 0.72 |
| 0.90 | 0.27 | 0.54 | 0.81 |
| 0.95 | 0.29 | 0.57 | 0.86 |

From this, the power is computed as a function of the sample size N and the fraction of individuals in which an abnormality is detected. To illustrate, an abnormality is “recurrent” if it is observed in at least 30% of samples. The power to detect a recurrent abnormality with N=10, 15, or 20 samples is listed in Table 3. Here, 20 samples have adequate power to detect markers with a penetrance of at least 60%.

TABLE 3
Power to detect a recurrent abnormality in at least 30% of samples as a
function of penetrance, detection probability, and sample size

| | N = 10 | | | N = 15 | | | N = 20 | | |
|---|---|---|---|---|---|---|---|---|---|
| Detec- | Penetrance = | | | | | | | | |
| tion | 0.30 | 0.60 | 0.90 | 0.30 | 0.60 | 0.90 | 0.30 | 0.60 | 0.90 |
| 0.50 | 0 | 1 | 10 | 4 | 13 | 55 | 2 | 39 | 87 |
| 0.60 | 0 | 3 | 24 | 1 | 27 | 80 | 5 | 62 | 97 |
| 0.70 | 0 | 7 | 46 | 2 | 45 | 94 | 11 | 80 | 99 |
| 0.80 | 0 | 14 | 70 | 5 | 64 | 99 | 18 | 92 | 100 |
| 0.90 | 0 | 24 | 90 | 8 | 80 | 100 | 28 | 97 | 100 |
| 0.95 | 1 | 31 | 96 | 11 | 86 | 100 | 33 | 99 | 100 |

In order to select a panel of markers, the marker with largest observed penetrance is the first. Markers are then added one at a time to maximize the additional independent information that they provide. Where multiple choices exist, markers that provide the easiest development of FISH assays were selected.

### Estimate Frequencies of Specific Abnormalities by FISH

For each abnormality, its presence or absence is treated as a binomial experiment. Bayesian methods were used, putting a uniform prior distribution on the frequency. Thus, if the abnormality is observed to be present in K out of N individuals, the posterior distribution on the frequency has a Beta (K+1, N−K+1) distribution. The mean of this distribution is μ=(K+1)/(N+2) and the standard deviation is σ=μ(1-μ/(N+3). An appropriate sample size was determined based on the desired accuracy of the estimated frequency.

TABLE 4
Posterior standard deviation of frequency estimates as a function
of the sample size N and the estimated frequency

| | Frequency | | | | |
|---|---|---|---|---|---|
| N | 0.5 | 0.6 | 0.7 | 0.8 | 0.9 |
| 10 | 0.139 | 0.136 | 0.127 | 0.111 | 0.083 |
| 20 | 0.104 | 0.102 | 0.096 | 0.083 | 0.063 |
| 30 | 0.087 | 0.085 | 0.080 | 0.070 | 0.052 |
| 40 | 0.076 | 0.075 | 0.070 | 0.061 | 0.046 |
| 50 | 0.069 | 0.067 | 0.063 | 0.055 | 0.041 |
| 60 | 0.063 | 0.062 | 0.058 | 0.050 | 0.038 |

### To Determine if Levels of the Markers are Different in Case/Control and in Different Stages of Lung Cancer.

The statistical analysis used two-sample t-tests (for case versus control) and analysis of variance (ANOVA) to compare different stages of lung cancer for each marker. Based on the data, the percentages of cells testing positive for a marker in the case and controls had means between roughly 1 and 10 and standard deviations roughly between 1 and 5. In order to have 80% power to detect, at the 5% significance level, a difference in percentages of at least 2 under these circumstances requires 17 samples in each group of a case/control design.

### Results

### 1. Develop and Validate a Sensitive FISH Biomarker Panel

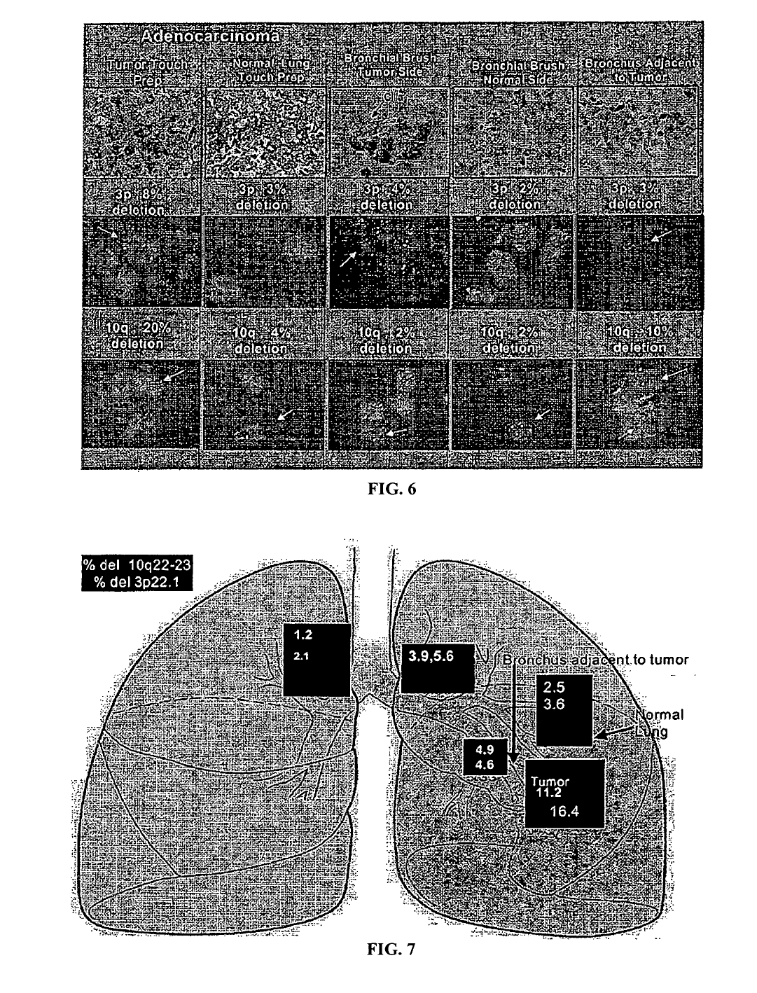

Using a cDNA comparative genomic hybridization (CGH) microarrays derived from a surgically resected set of 14 primary non-small cell lung cancers (6 adenocarcinomas and 8 squamous carcinomas), a set of novel DNA probes were developed for 3p22.1 (GC20 or Sui homolog) and 10q22-23 (surfactant protein A) that are deleted in the majority of lung cancers tested (Jiang et al., 2004). The inventors demonstrated by FISH using an extensive mapping strategy on lung tissues from patients with resected early stage lung cancer, that these probes demonstrated a field cancerization effect involving both morphologically normal and malignant tissues in both smokers and non-smokers who develop lung cancer (Li et al., 2006; Jiang and Katz, 2002; Barkan et al., 2004); Bubendorf et al., 2005) (FIG. 7). The inventors further demonstrated these same molecular changes exist to a much higher degree in the corresponding primary lung cancers (FIG. 6). The inventors also showed that deletion of these biomarkers in tumors and adjacent bronchial tissue is significantly associated with the presence of lymph node metastases as well as decreased survival in early stage lung cancer. Additionally, extensive studies in sputum from patients with early lung cancer and high-risk controls have demonstrated these same FISH marker to be deleted in both normal and dysplastic epithelial cells, neutrophils and macrophages. Compared to the controls, patients with lung cancer have significantly higher levels of deletions of these markers (P<0.0001). The inventors have further demonstrated in a pilot chemo-prevention study in sputum from patients at high risk to develop lung cancer, that the proposed markers are highly sensitive surrogate biomarkers to monitor progressive eradication of neoplastic clones (both in epithelial cells and neutrophils) in the sputum by FISH.

In a pilot blinded study of blood received from patients with lung cancer and patients at high risk for lung cancer, the inventors demonstrated, in mononuclear cells, using Ficoll-Hypaque purified blood, a high degree of clonally related chromosomal abnormalities for the probes 3p22.1 and 10q22-23. These occurred at significantly higher levels predominantly in non-fluorescent or CD45-negative cells in cancer patients as compared to controls (Tables 5 and 6, below).

TABLE 5

| | | | | | Std. |
|---|---|---|---|---|---|
| | | | % Mean | Std. | Error |
| | Diagnosis | N | deletions | Deviation | Mean |
| 10q Del | No Cancer | 8 | 1.88 | 1.126 | .398 |
| | Cancer | 8 | 4.63 | 1.847 | .653 |
| Cen 10 Del | No Cancer | 8 | 1.25 | .886 | .313 |
| | Cancer | 8 | 4.13 | 3.137 | 1.109 |
| Neutrophils | No Cancer | 8 | .38 | .518 | .183 |
| 10q Del | Cancer | 8 | 13 | .354 | .125 |
| Polysomies | No Cancer | 8 | 1.25 | .707 | .250 |
| 10 | Cancer | 8 | 2.50 | 2.507 | .886 |
| Total | No Cancer | 8 | 3.50 | 1.927 | .681 |
| Cen10/10q | Cancer | 8 | 8.88 | 2.997 | 1.060 |
| Del | | | | | |
| Cen10/10q | No Cancer | 4 | 8.00 | 5.477 | 2.739 |
| Del | | | | | |
| 3p Del | No Cancer | 8 | 5.00 | 1.604 | .567 |
| | Cancer | 7 | 7.43 | 1.902 | .719 |
| Cen 3 Del | No Cancer | 8 | .38 | .744 | .263 |
| | Cancer | 7 | .71 | .951 | .360 |
| Neutrophils | No Cancer | 8 | 1.63 | 2.066 | .730 |
| 3p Del | Cancer | 7 | .43 | .787 | .297 |
| Polysomies 3 | No Cancer | 8 | 6.25 | 3.012 | 1.065 |
| | Cancer | 7 | 6.00 | 2.708 | 1.024 |
| Total Cen | No Cancer | 8 | 7.00 | 4.071 | 1.439 |
| 3/3p Del | Cancer | 7 | 8.57 | 2.370 | .896 |
| Cen 3/3p | No Cancer | 2 | 20.50 | 6.364 | 4.500 |
| Cen 3 | No Cancer | 5 | 2.60 | 1.140 | .510 |
| | Cancer | 6 | 2.50 | 3.017 | 1.232 |
| Cen 7 | No Cancer | 5 | .20 | .447 | 200 |
| | Cancer | 6 | .67 | .816 | .333 |
| Cen 17 | No Cancer | 5 | 1.00 | .707 | .316 |
| | Cancer | 6 | 1.17 | 2.041 | .833 |
| LSI 9p21 | No Cancer | 5 | .80 | .837 | .374 |
| | Cancer | 6 | 2.33 | 2.805 | 1.145 |

Abbreviations:
10qdel = deletions of 10q22-23,
cen10 = centromeric 10,
3p del = deletions of 3p22.1,
cen 3 = centromeric 3,
cen 7 = centromeric 7,
cen 17 = centromeric 17,
LSI = locus specific identifer.

TABLE 6
Significant differences found in percentage of deletions for 3p and 10 in cases with lung cancer compared
to controls

| | | | | t-test for Equality of Means | | | | | | |
|---|---|---|---|---|---|---|---|---|---|---|
| | | Levene's Test | | | | | | | 95% Confidence | |
| | | for Equality of | | | | Sig. | | Std. | Interval of the | |
| | | Variances | | | | (2- | Mean | Error | Difference | |
| | | F | Sig. | t | df | tailed) | Difference | Difference | Lower | Upper |
| 10q | Equal | 1.217 | 288 | −3.596 | 14 | .003 | −2.750 | 765 | −4.390 | −1.110 |
| Del | variances | | | | | | | | | |
| | assumed | | | | | | | | | |
| | Equal | | | −3.596 | 11.572 | .044 | −2.750 | .765 | −4.423 | −1.077 |
| | variances | | | | | | | | | |
| | not | | | | | | | | | |
| Cen | Equal | 13.347 | .003 | −2.495 | 14 | .026 | −2.875 | 1.152 | −5.347 | −.403 |
| 10 | variances | | | | | | | | | |
| Del | assumed | | | | | | | | | |
| | Equal | | | −2.495 | 8.111 | .037 | −2.875 | 1.152 | −5.526 | −.224 |
| | variances | | | | | | | | | |
| | not | | | | | | | | | |
| Total | Equal | 716 | .412 | −4.267 | 14 | 001 | −5.375 | 1.260 | −8.077 | −2.673 |
| 10q/ | variances | | | | | | | | | |
| 10cent | assumed | | | | | | | | | |
| Del | | | | | | | | | | |
| | Equal | | | −4.267 | 11.944 | .001 | −5.375 | 1.260 | −8.121 | −2.629 |
| | variances | | | | | | | | | |
| | not | | | | | | | | | |
| 3p | Equal | .031 | 862 | −2.685 | 13 | .019 | −2.429 | .905 | −4.383 | −474 |
| Del | variances | | | | | | | | | |
| | assumed | | | | | | | | | |
| | Equal | | | −2.652 | 11.853 | .021 | −2.429 | .916 | −4.426 | −.431 |
| | variances | | | | | | | | | |
| | not | | | | | | | | | |

The inventors had previously shown that these same abnormalities commonly exist in the majority of non-small cell carcinomas in lung and adjacent morphologically normal tissue on the same side but not the contra-lateral side of the carcinoma (FIG. 6). These probes (3p and 10q) moreover were tested in conjunction with commercially available probes for chromosomes 3, 7, 17 and 9p21.3. The inventors showed that none of these commercial probes manifested significant abnormalities and the levels of these abnormalities were not significantly different from control blood samples (Table 5).

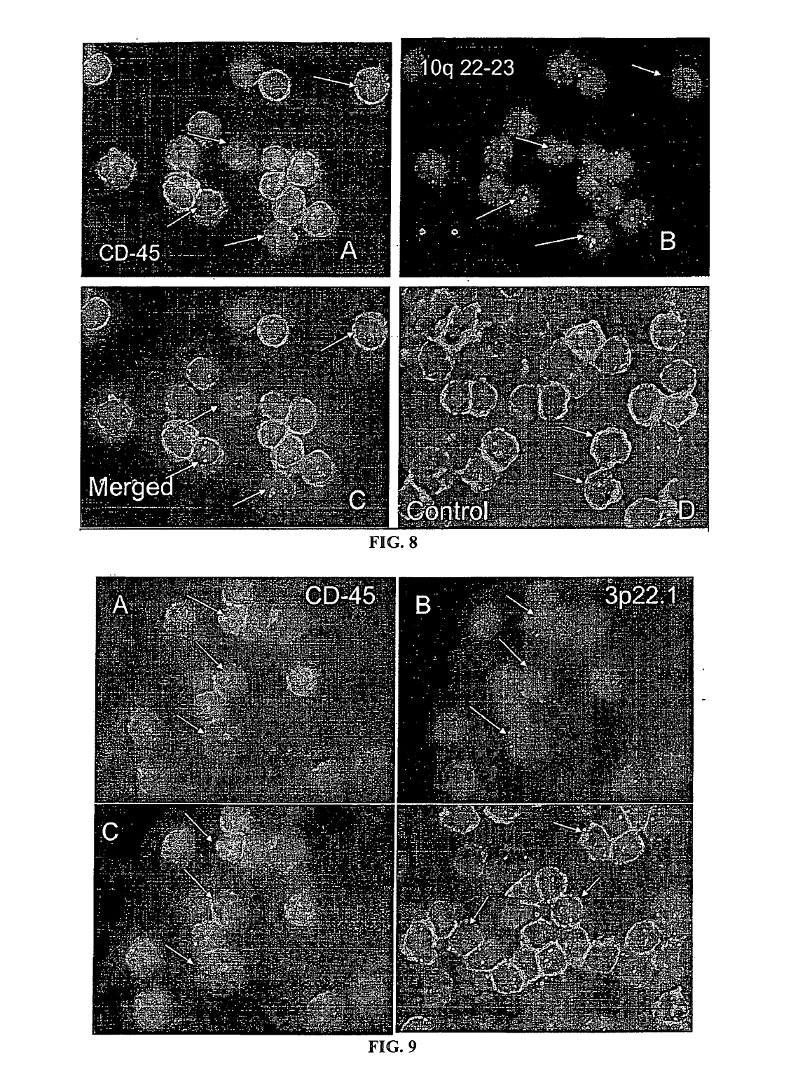

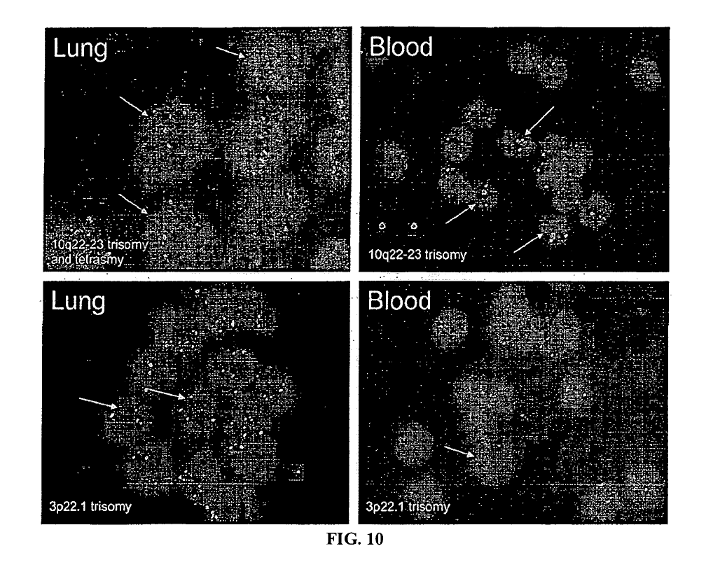

Quantitatively the inventors found that the blood samples collected from lung patients contained large numbers of CD45-negative cells with clonal abnormalities (FIG. 8), Morphologically these cells had slightly larger nuclei (larger than mature lymphocytes), with less condensed cytoplasm, however on a regular Romanowsky stained specimen of blood these cells closely resemble normal lymphocytes or monocytes. Some of these cells have nuclear indentations or coffee bean shaped indentations and more abundant cytoplasm (FIGS. 8 and 9). Compared to reports of isolating tumor cells with magnetic immuno-coated cytokeratin beads, it appears that the inventors have discovered much larger quantities of abnormal cells. Staining with pan-cytokeratin fluorescent antibodies revealed 30% positive cells in the peripheral blood of the lung cancer patients similar to the percentage of CD45-negative cells. This finding is well illustrated in FIG. 12, which depicts around 30% of CTCs, which were stained positive for cytokeratin (FIG. 12B) and negative for CD45 (FIG. 12A). This blood specimen was obtained from a 69 year-old male, who at the time that his blood was procured had Stage 1V non-small cell (squamous) carcinoma, which was progressing.

In two blood specimens from patients, both with limited small cell lung cancer (stageT1N1MO and stage T2N2MO) CD45, negative cells comprised 30% and 41 5% respectively of the total mononuclear cell population, of which chromosomal abnormalities (demonstrated by the FISH, probes for 10q22-23 and 3p22.1) were noted in 62-64% of CD45-negative cells (FIGS. 8 and 9). If the inventors assume that there were a total of 1 million isolated mononuclear cells from 1 ml of whole blood (conservative estimate), then between 180,000 to 240,000 of non-fluorescent cells with clonally related chromosomal abnormalities (CTCs) were present in 1 ml of peripheral blood and 1,800,000 and 2,400,000 CTCs were present in 10 ml of blood.

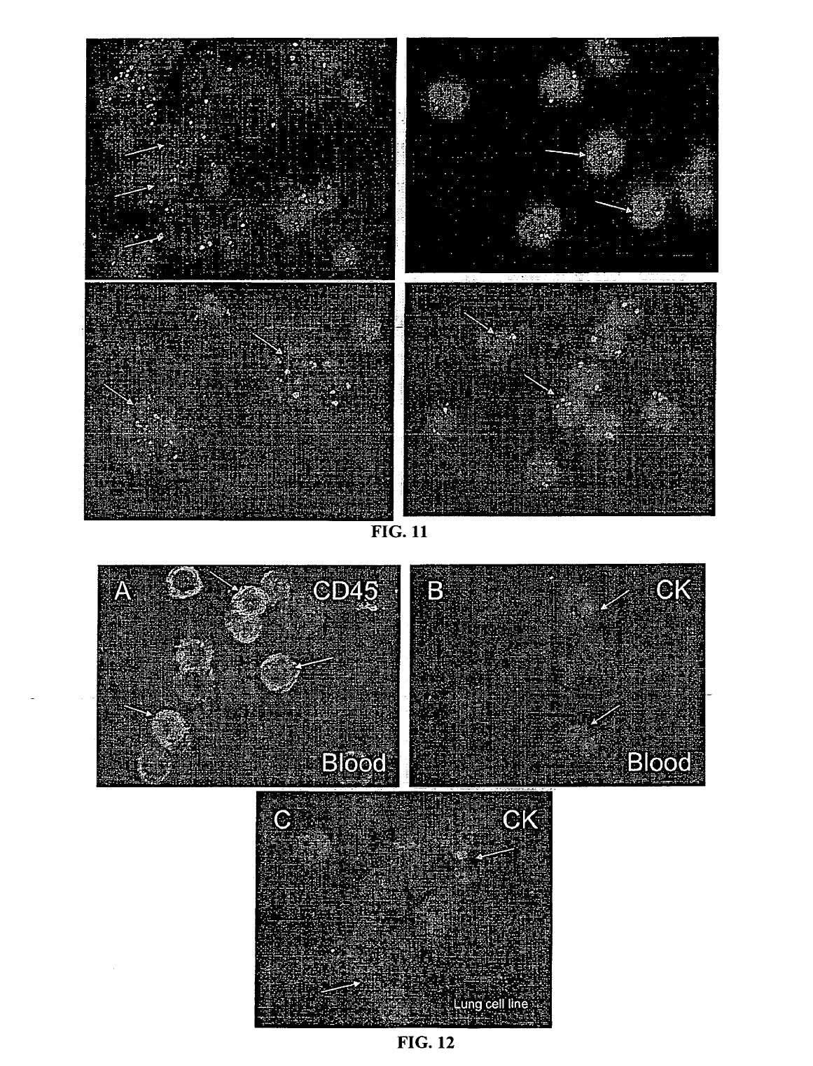

Similarly in FIG. 11, top right panel, also from another patient with limited small cell carcinoma (T2N2MO) clonally abnormal cells with deletion of the epidermal growth factor receptor (EGFR) gene, which were present in 10% of total mononuclear cells, are depicted, while the bottom right panel shows some CTCs with extra copies of EGFR. Similar EGFR abnormalities by FISH can occur in lung cancer and the top and bottom left panels depict a range of EGFR abnormalities in touch imprints of other patients with non-small cell or adenocarcinoma of lung.

These findings are in sharp contrast to the report by Christofanilli et al. (2004) who used a system which enriches 10 ml of blood for cells expressing an epithelial-cell adhesion molecule with antibody-coated magnetic beads (Cellsearch System, Veridex) and who defined CTC as nucleated cells lacking CD45 and expressing cytokeratin. The prevalence of CTCs as defined above, in the breast cancer population with metastatic disease at baseline ranged between 2 and >1000 per 10 ml of blood, with 94% of these patients having equal to or less than 50 CTC per 10 ml of blood. The large differences in levels of CTCs noted in the patients of the current study versus the breast cancer patients may be attributed to different cancer sub types with different biology (breast versus lung cancer), different stage of disease and most importantly, different methodology in assaying for levels of CTCs.

Most interesting are the findings of patients with “limited” small cell carcinomas of lung, who have such high levels of CTCs, yet had not at the time of blood collection, manifested metastases. It would appear that such patients would benefit enormously from systemic therapies that should be continued until CTCs have cleared.

### 2. Tests of Genetic Susceptibility

Tobacco smoke carcinogens and individual susceptibility play key roles in determining risk of lung cancer. There are a variety of biomarkers evaluating susceptibility to the carcinogenic effects of benzo[a]pyrene; however, no assays specifically evaluate susceptibility to the nicotine-derived nitrosamine 4-(methylnitrosamino)-1-(3-pyridyl)-1-butanone (NNK), a potent inducer of lung adenocarcinoma. In this case-control study, the inventors modified the cytokinesis-block micronucleus (CBMN) assay, an established biomarker for DNA damage and genomic instability, to evaluate susceptibility to NNK by measuring the frequency of NNK-induced chromosomal damage endpoints (micronuclei [MN]; nucleoplasmic bridges [NPBs] and nuclear buds “BUDS]) per 1000 binucleated peripheral blood lymphocytes. Levels of both spontaneous and NNK-induced chromosomal damage were significantly higher in lymphocytes from 139 lung cancer patients than those from 130 matched controls. Forty-seven percent of the cases (compared with 12% of the controls) had ≧4 spontaneous MN, 66% of the cases (and no controls) had ≧4 spontaneous NPBs, and 25% of cases (versus 5% of controls) had ≧1 spontaneous NBUDs (P<001). Similarly, 40% of the cases (versus 6% of the controls) had ≧5 NNK-induced MN, 89% of the cases (and no controls) had ≧6 induced NPBs, and 23% of the cases (vs 2% of the controls had 2 induced NBUDs (P<001). As continuous variables, spontaneous MN, NPBs and NBUDs were associated with 2-, 29-, and 6-fold increases in risk for cancer. Similarly, NNK-induced 2 3-, 45.5-, and 10-fold increases in risk for cancer. The inventors also evaluated the use of results from the CBMN assay to predict cancer risk based on the numbers of MN, NPBs, and NBUDs defined by percentile cut-points in control data. The probability of being a cancer patient were 96%, 98% and 100% when using the 95^{th} percentiles of spontaneous and NNK-induced MN, NPBs and NBUDs, respectively, in combination. The study indicates that the CBMN assay is extremely sensitive to NNK-induced genetic damage and that the results provide a strong predictor of lung cancer risk. Table 5 indicates the mean and standard deviations of percentage deletions of genes compared to an internal reference in all peripheral blood mononuclear cells in cancer patients and high risk control patients. It is noted that counts were performed on all mononuclear cells in a simple buffy coat, and not enriched for CD45-negative cells by a Ficoll. Table 2 indicates the statistically significant differences identified.

### Example 2

### Staging by Circulating Tumor Cells

Biomarker Abnormalities Associated with Tumor Stage

To investigate if any of the markers can be used to differentiate between the different pathological stages of disease compared to controls, a univariate multinomial logistic regression (with controls as the reference group) was fit for each marker, separately. Table 7 displays those stages that are significantly associated (P<0.05) with each marker (denoted by X).

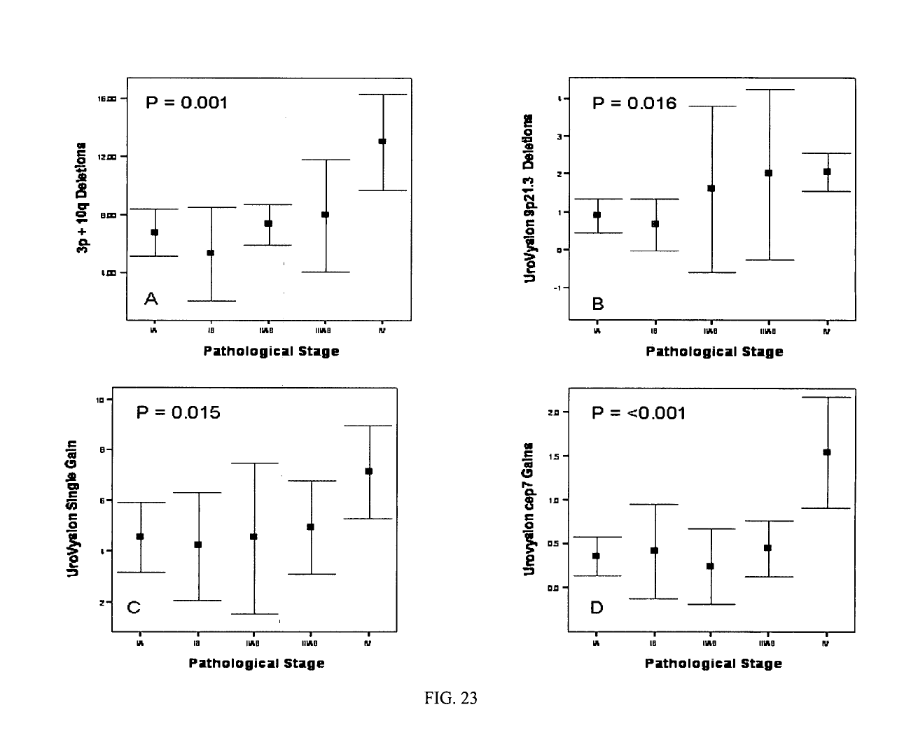

Some of the CACs were significantly associated with early-stage (IA) and/or late-stage (IIIA, IIIB, or IV) NSCLC (P<0.05). Table 7. Most notable were CACs containing abnormalities of 3p22.1/CEP3 and 10q22.3/CEP10, and gain or loss of biomarkers in the URO set (FIG. 23). CTCs with at least two abnormalities of URO or LAV, or abnormalities of 3p22.1/CEP3 or gains of 10q22.3/CEP10 correlated with late disease stage.

TABLE 7
Markers Associated (P < 0.05) with Pathological Stage of Disease
Compared to Controls

| Markers | | IA | IB | II | III | IV |
|---|---|---|---|---|---|---|
| 3p22.1 | Deletions | X | — | X | X | X |
| | Abnormalities/CEP3 | X | — | X | X | X |
| | Mono CEP | — | — | — | — | X |
| | Poly CEP | X | X | X | X | X |
| | Combined | X | X | X | X | X |
| 10q22.3 | Deletions | X | X | X | X | X |
| | Abnormalities/CEP10 | X | — | X | X | X |
| | Mono CEP | X | X | — | X | X |
| | Poly CEP | X | X | X | X | X |
| | Combined | X | X | X | X | X |
| 3p22.1/10q22.3 | Deletions | X | X | X | X | X |
| LaVysion Deletions | Single | X | X | X | X | X |
| | EGFR | X | — | — | X | X |
| | 5p15.2 | X | X | X | X | X |
| | C-myc | X | — | — | X | X |
| | 6p11-q11 | — | — | — | — | — |
| LaVysion Gains | Single | X | — | — | X | X |
| | EGFR | X | — | — | X | X |
| | 5p15.2 | — | — | — | — | X |
| | C-myc | — | — | — | — | — |
| | 6p11-q11 | — | — | — | — | X |
| LaVysion % Abnormalities | | X | X | | X | X |
| UroVysion Deletions | Single | X | X | X | X | X |
| | CEP3 | — | — | — | — | — |
| | CEP7 | X | X | X | X | X |
| | 9p21.3 | — | — | — | X | X |
| | CEP 17 | — | — | X | — | — |
| UroVysion Gains | Single | X | X | X | X | X |
| | CEP3 | X | — | X | X | X |
| | CEP7 | — | — | — | — | X |
| | 9p21.3 | — | — | X | X | — |
| | CEP17 | X | — | — | — | X |
| UroVysion | | — | X | X | X | X |
| % Abnormalities | | | | | | |
| URO + LAV Abnormalities | | x | x | x | x | x |
| Circulating Tumor Cells | | X | X | — | X | X |
| Urovysion | | X | X | X | X | X |
| | LaVysion | X | — | — | X | X |
| | Deletions 3p22.1 | X | X | X | X | X |
| | Gains 3p22.1/CEP3 | X | X | X | X | X |
| | Deletions 10q22.3 | — | — | — | X | X |
| | Gains 10q22.3/CEP10 | | | | | |

### CACs and CTCs by Case, Control Status and NSCLC Stage

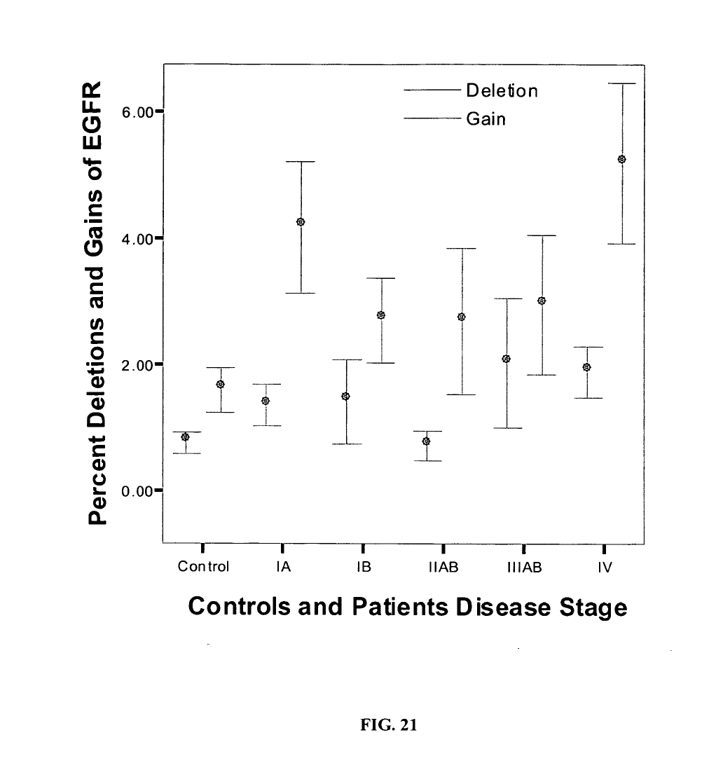

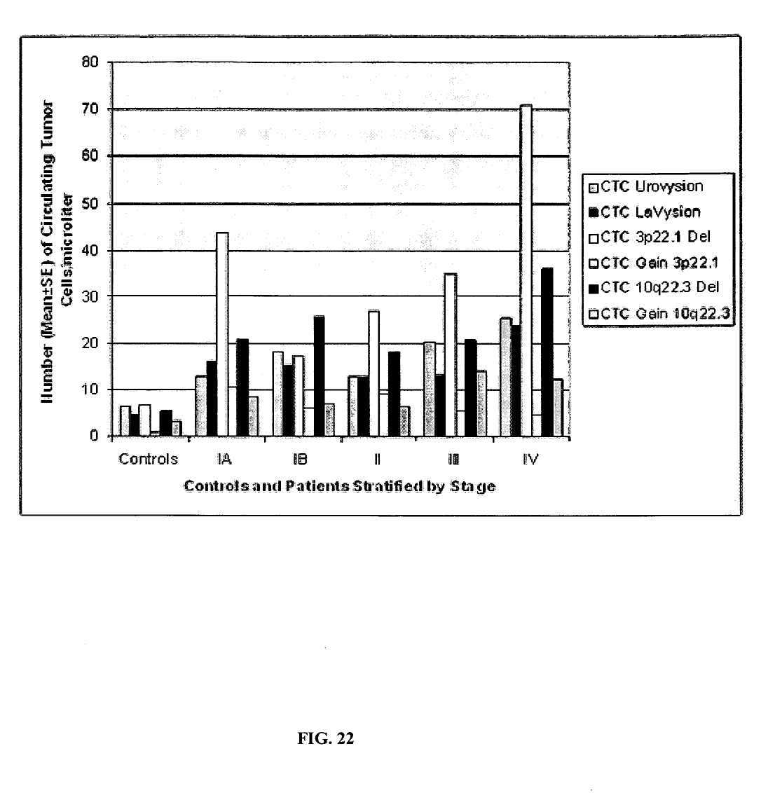

The mean percentage of circulating cytogenetically abnormal cells (CACs) in patients and controls were recorded and stratified by pathological stage (following tumor resection) and clinical stage in cases that were inoperable because of a high stage (IIIB or IV). There was a significant trend for percentages of CACs for all biomarkers, except the LAV probe set, to increase from low stage to high stage disease. Highly significant differences were noted in the biomarker distribution between the patients and controls (Table 8). For example, the mean±standard error of the mean [SEM] percentage of CACs in the controls ranged from 0.17±0.07 for 3p22.1/CEP3 gains to 3.05±0.46 for combined 3p22.1/CEP3 chromosomal abnormalities. In comparison, the mean±SEM percentage of CACs for patients with stage 1A NSCLC ranged from 1.11±0.30 for 3p22.1/CEP3 gains to 7.00±0.93 for combined 3p22.1/CEP3 abnormalities. Both EGFR deletions and gains were significantly different between cases and controls (FIG. 21 and Table 8).

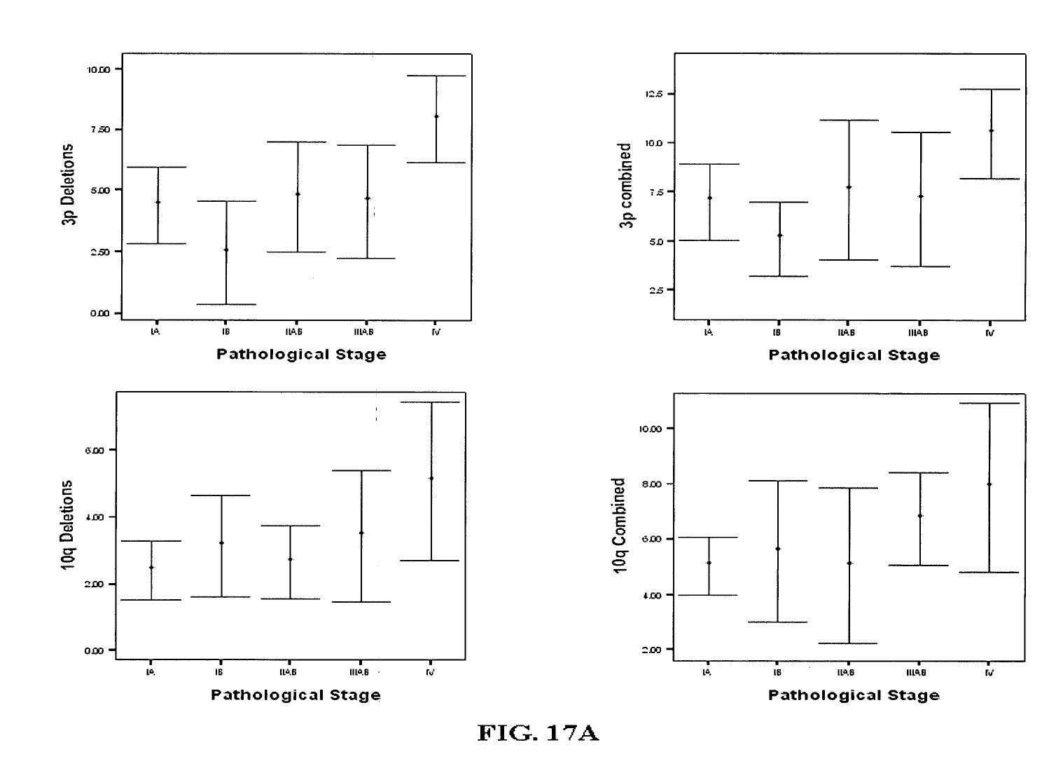

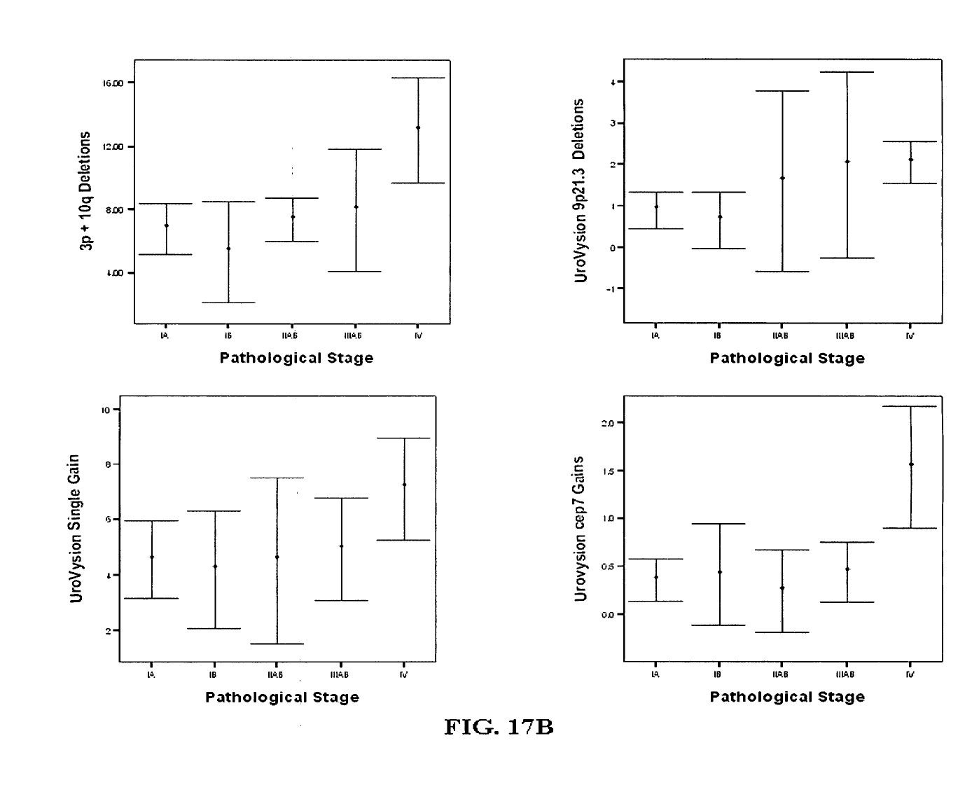

Similarly the mean numbers of CTCs per microliter (derived from the percentages of CACs) were significantly different compared to controls for all biomarker abnormalities recorded. Expressed per milliliter the mean number of CTCs for all cases of NSCLC ranged from 7,230±1320 for deletions of 10q22.3/CEP10 to 45,520±7490 for deletions of 3p22.1/CEP3, while for URO and LAV abnormalities mean CTCs were 18,790±3160 and 17570±2820 respectively (FIGS. 17A-B, 18A-H, and 22; Table 9).

TABLE 8
Distribution of Biomarkers in PBMCs (CACs) Stratified by Case/Control Status and Tumor Stage

| | | | Cases Stratified by Pathological Stage | | | | | | |
|---|---|---|---|---|---|---|---|---|---|
| Marker % | Controls | Cases | IA | IB | II | III | IV | P | |
| (Mean ± SE) | N = 24 | N = 59 | N = 16 | N = 8 | N = 7 | N = 10 | N = 18 | MW^{a} | trend^{b} |
| deletion 3p22.1/CEP3 | 2.25 ± 0.40 | 5.33 ± 0.46 | 4.40 ± 0.73 | 2.48 ± 0.85 | 4.73 ± 0.91 | 4.55 ± 1.02 | 7.95 ± 0.85 | <0.001 | 0.001 |
| gains 3p22.1/CEP3 | 0.17 ± 0.07 | 0.79 ± 0.11 | 1.11 ± 0.30 | 0.57 ± 0.22 | 0.84 ± 0.30 | 0.78 ± 0.17 | 0.58 ± 0.18 | <0.001 | 0.124 |
| mono 3p22.1/CEP3 | 0.36 ± 0.13 | 0.66 ± 0.09 | 0.50 ± 0.11 | 0.69 ± 0.44 | 0.78 ± 0.17 | 0.57 ± 0.18 | 0.89 ± 0.15 | 0.023 | 0.162 |
| poly 3p22.1/CEP3 | 0.28 ± 0.12 | 1.18 ± 0.17 | 0.99 ± 0.23 | 1.37 ± 0.41 | 0.54 ± 0.38 | 1.27 ± 0.45 | 1.10 ± 0.24 | <0.001 | 0.868 |
| Combined abnormalities | 3.05 ± 0.46 | 7.96 ± 4.37 | 7.00 ± 0.93 | 5.10 ± 0.78 | 7.62 ± 1.48 | 7.17 ± 1.51 | 10.52 ± 1.09 | <0.001 | 0.011 |
| deletion 10q22.3/CEP10 | 0.76 ± 0.21 | 3.52 ± 0.42 | 2.30 ± 0.41 | 3.13 ± 0.64 | 2.65 ± 0.45 | 3.44 ± 0.88 | 5.08 ± 1.13 | <0.001 | 0.017 |
| gains 10q22.3/CEP10 | 0.43 ± 0.11 | 1.20 ± 0.18 | 1.00 ± 0.22 | 0.87 ± 0.27 | 1.53 ± 0.83 | 1.34 ± 0.51 | 1.30 ± 0.39 | 0.003 | 0.420 |
| mono 10q22.3CEP10 | 0.47 ± 0.16 | 1.11 ± 0.14 | 1.23 ± 0.25 | 1.18 ± 0.40 | 0.39 ± 0.20 | 1.23 ± 0.36 | 1.18 ± 0.31 | 0.005 | 0.959 |
| pol 10q22.3/CEP 10 | 0.01 ± 0.01 | 0.44 ± 0.07 | 0.41 ± 0.10 | 0.46 ± 0.18 | 0.46 ± 0.18 | 0.73 ± 0.27 | 0.32 ± 0.12 | <0.001 | 0.995 |
| Combined abnormalities | 1.67 ± 0.24 | 6.27 ± 0.53 | 5.03 ± 0.49 | 5.58 ± 1.08 | 2.04 ± 1.14 | 3.74 ± 0.72 | 7.89 ± 1.44 | <0.001 | 0.030 |
| 3p22.1 and 10q22.3 | 3.01 ± 0.52 | 8.83 ± 0.72 | 6.79 ± 0.76 | 5.34 ± 1.29 | 7.39 ± 0.57 | 7.99 ± 1.69 | 13.03 ± 1.57 | <0.001 | <0.001 |
| Deletions | | | | | | | | | |
| LaVysion Deletions | | | | | | | | | |
| Single | 4.41 ± 0.63 | 7.31 ± 0.51 | 6.30 ± 0.67 | 7.54 ± 1.88 | 7.08 ± 1.06 | 9.24 ± 1.59 | 7.08 ± 0.87 | 0.001 | 0.409 |
| EGFR | 0.77 ± 0.12 | 1.54 ± 0.18 | 1.35 ± 0.23 | 1.34 ± 0.45 | 0.57 ± 0.20 | 2.32 ± 0.67 | 1.75 ± 0.30 | 0.007 | 0.178 |
| 5p15.2 | 0.45 ± 0.17 | 1.85 ± 0.27 | 1.55 ± 0.72 | 1.91 ± 0.83 | 1.76 ± 0.41 | 2.10 ± 0.71 | 1.95 ± 0.38 | <0.001 | 0.563 |
| C-myc | 0.15 ± 0.06 | 0.64 ± 0.10 | 0.60 ± 0.16 | 0.46 ± 0.15 | 0.23 ± 0.15 | 0.85 ± 0.36 | 0.78 ± 0.21 | 0.002 | 0.294 |
| 6p11-q11 | 2.99 ± 0.66 | 3.41 ± 0.35 | 2.83 ± 0.51 | 3.83 ± 1.00 | 4.54 ± 1.13 | 3.94 ± 0.79 | 2.96 ± 0.73 | 0.256 | 0.930 |
| LaVysion Gains | | | | | | | | | |
| Single | 2.66 ± 0.37 | 5.85 ± 0.50 | 5.83 ± 0.75 | 4.49 ± 0.66 | 3.77 ± 0.79 | 4.42 ± 0.83 | 8.34 ± 1.22 | <0.001 | 0.065 |
| EGFR | 1.60 ± 0.26 | 3.86 ± 0.39 | 4.26 ± 0.71 | 2.50 ± 0.42 | 2.34 ± 0.63 | 3.32 ± 0.86 | 5.01 ± 0.89 | <0.001 | 0.341 |
| 5p15.2 | 0.35 ± 0.12 | 0.68 ± 0.12 | 0.52 ± 0.12 | 0.70 ± 0.38 | 0.66 ± 0.39 | 0.25 ± 0.11 | 1.06 ± 0.27 | 0.100 | 0.207 |
| C-myc | 0.14 ± 0.08 | 0.22 ± 0.06 | 0.09 ± 0.05 | 0.47 ± 0.32 | 0.14 ± 0.12 | 0.10 ± 0.07 | 0.32 ± 0.12 | 0.247 | 0.433 |
| 6p11-q11 | 0.65 ± 0.16 | 1.02 ± 0.14 | 0.97 ± 0.35 | 0.45 ± 0.25 | 0.77 ± 0.40 | 0.94 ± 0.39 | 1.45 ± 0.21 | 0.180 | 0.121 |
| All LaVysion | 0.65 ± 0.22 | 2.18 ± 0.26 | 1.63 ± 0.25 | 2.12 ± 0.13 | 1.26 ± 0.34 | 2.33 ± 0.65 | 2.92 ± 0.65 | <0.001 | 0.057 |
| Abnormalities | | | | | | | | | |
| UroVysion Deletions | | | | | | | | | |
| Single | 5.36 ± 0.75 | 8.31 ± 0.43 | 7.35 ± 0.69 | 8.06 ± 1.62 | 10.2 ± 0.90 | 8.60 ± 1.13 | 8.3.8 ± 0.77 | <0.001 | 0.355 |
| CEP3 | 0.20 ± 0.06 | 0.21 ± 0.06 | 0.13 ± 0.10 | 0.05 ± 0.05 | 0.11 ± 0.11 | 0.19 ± 0.17 | 0.39 ± 0.12 | 0.547 | 0.051 |
| CEP7 | 0.15 ± 0.06 | 1.06 ± 0.18 | 0.69 ± 0.20 | 1.47 ± 0.93 | 1.05 ± 0.24 | 1.22 ± 0.30 | 1.13 ± 0.37 | <0.001 | 0.474 |
| 9p21.3 | 0.89 ± 0.21 | 1.49 ± 0.23 | 0.90 ± 0.21 | 0.66 ± 0.29 | 1.60 ± 0.90 | 2.00 ± 0.99 | 2.06 ± 0.23 | 0.093 | 0.016 |
| CEP 17 | 4.17 ± 0.33 | 5.54 ± 0.33 | 5.63 ± 0.55 | 5.88 ± 1.61 | 7.31 ± 0.90 | 5.21 ± 0.37 | 4.82 ± 0.48 | 0.008 | 0.266 |
| UroVysion Gains | | | | | | | | | |
| Single | 2.23 ± 0.31 | 5.35 ± 0.42 | 4.54 ± 0.65 | 4.20 ± 0.90 | 4.52 ± 1.22 | 4.92 ± 0.82 | 7.13 ± 0.88 | <0.001 | 0.015 |
| CEP3 | 1.22 ± 0.19 | 3.26 ± 0.32 | 2.99 ± 0.51 | 1.97 ± 0.59 | 3.35 ± 1.16 | 3.27 ± 0.83 | 4.02 ± 0.65 | <0.001 | 0.130 |
| CEP7 | 0.52 ± 0.14 | 0.73 ± 0.13 | 0.35 ± 0.10 | 0.41 ± 0.22 | 0.24 ± 0.18 | 0.44 ± 0.14 | 1.54 ± 0.30 | 0.466 | <0.001 |
| 9p21.3 | 0.23 ± 0.10 | 0.64 ± 0.10 | 0.56 ± 0.15 | 0.82 ± 0.54 | 0.85 ± 0.41 | 0.61 ± 0.19 | 0.58 ± 0.11 | 0.003 | 0.889 |
| CEP17 | 0.26 ± 0.07 | 0.64 ± 0.10 | 0.65 ± 0.16 | 0.52 ± 0.29 | 0.08 ± 0.07 | 0.44 ± 0.17 | 1.02 ± 0.22 | 0.047 | 0.196 |
| All UroVysion | 0.77 ± 0.13 | 2.11 ± 0.19 | 1.32 ± 0.32 | 2.27 ± 0.44 | 2.33 ± 0.56 | 2.35 ± 0.42 | 2.55 ± 0.38 | <0.001 | 0.018 |
| Abnormalities | | | | | | | | | |
| Uro and LaV % | 1.42 ± 0.291 | 4.29 ± 0.37 | 2.86 ± 0.47 | 4.38 ± 0.50 | 3.60 ± 0.65 | 4.69 ± 0.86 | 5.48 ± 0.88 | <0.001 | 0.008 |
| Abnormal | | | | | | | | | |

P-value derived from Mann-Whitney test (all cases vs. controls);
^{b}P-value for trend of abnormality across tumor stage;
all P-values are two-sided per *microliter.

TABLE 9

| | Cases [symbol] by Pathological Stage | | | | | | | | |
|---|---|---|---|---|---|---|---|---|---|
| Circulating | Controls | IA | IB | II | III | IV | Cases | P | |
| Tumor Cells | N = 24 | N = 16 | N = 8 | N = 7 | N = 10 | N = 18 | N = 59 | MW^{a} | trend^{b} |
| UroVysion | 6.31 ± 1.19 | 13.04 ± 3.30 | 18.37 ± 5.39 | 13.10 ± 5.86 | 20.44 ± 7.00 | 25.37 ± 6.58 | 18.79 ± 3.16 | 0.006 | 0.144 |
| LaVysion | 4. [symbol] ± 1.37 | 18.19 ± 3.43 | 15.10 ± 3.76 | 12.00 ± 7.50 | 13.48 ± 3.03 | 23.89 ± 7.82 | 17.57 ± 2.62 | <0.001 | 0.349 |
| 3p22.1 Del/cep 3 | 7.04 ± 2.81 | 43.75 ± 10.58 | 17.28 ± 6.87 | 27.09 ± 11.95 | 34.94 ± 10.35 | 71.12 ± 19.82 | 45.52 ± 7.49 | 0.011 | 0.13 |
| Gain 3p22.1/cep 3 | 1.12 ± 0.50 | 10.65 ± 3.11 | 6.06 ± 2.84 | 9.30 ± 5.09 | 5.56 ± 1.61 | 4.76 ± 2.13 | 7.23 ± 1.32 | <0.001 | 0.094 |
| 10q22.3/cep 10 Del | 5.58 ± 1.44 | 20.72 ± 16.25 | 25.74 ± 7.90 | 18.15 ± 6.87 | 20. [symbol] ± 4.93 | 35.98 ± 8.52 | 25.78 ± 3.28 | <0.001 | 0.124 |
| Gain 10q22.3/ | 3.44 ± 0.95 | 6.45 ± 1.70 | 7.08 ± 3.18 | 6.52 ± 2.33 | 14.22 ± 7.92 | 12.38 ± 5.06 | 10.21 ± 2.12 | 0.013 | 0.328 |
| cep 10 | | | | | | | | | |

P-value derived from Mann-Whitney test (all cases vs. controls);
^{b}P-value for trend of abnormality across tumor stage;
all P-values are two sided per microliter (To express number per [symbol] , multiply by 1000).
Abbreviations:
NSCLC—Non-Small Cell Lung Cancer,
CTC—Circulating Tumor Cells;
URO - UroVysion;
LAV - LAVysion;
del—Deletion [symbol]
indicates data missing or illegible when filed

There was a highly significant difference between biomarkers for cases versus controls based on numbers of CTCs detected by 4 sets of biomarkers. Most significant biomarkers were CTC/μl according to UroVysion, abnormalities of 3p/cep3, deletion 10q and polysomies 10 and 10q. Moreover with only two probe sets, 10q22.3/10 deletions and polysomies, and number of abnormal cells detected by UroVysion, it was possible to predict case versus control status, with an ROC of 0.97. This translates to a sensitivity and specificity of 97% to predict cancer status.

When comparing controls to early stage lung cancer stage 1A, the most significant biomarkers were CTC/μl according to UroVysion and LaVysion probe sets, ND abnormalities of 3p/cep3, deletion 10q and polysomies 10 and 10q. Similar findings were discovered comparing controls versus stage HA and IIB.

The results also demonstrated that different markers can be used to differentiate different stages from controls (Tables 7 and 8). For example “9p21.3_Uro_del” is the top predictor to tell apart stage 4 from controls and in the same time it is the last in the list of predictors of the stage 1A. Other markers, e.g. “EGFR_Lav_gain” are important both for stage 1A and stage 1V.

TABLE 10
Original Stage
Circulating Cell Count

| | Number of Cases | UroVysion | LaVysion |
|---|---|---|---|
| Controls | 15 | 4.02 | 4.2 |
| Stage IA | 13 | 15.56 | 11.00 |
| Stage IB | 7 | 17.53 | 16.74 |
| Stage IIA-IIB | 8 | 23.32 | 17.97 |
| Stage IIIA-IIIB | 9 | 11.67 | 7.79 |
| Stage IV | 9 | 31.75 | 43.60 |

TABLE 11
Molecular Staging by Circulating Tumor Cells*

| | Original Stage | | | Reclassified Stage After | | |
|---|---|---|---|---|---|---|
| | Prior to Surgery | | | Surgery | | |
| | Mean Circulating | | | Mean Circulating | | |
| | Cell Count/μl | | | Cell Count/μl | | |
| | Number of | | | Number of | | |
| | Cases | UroVys | LaVys | Cases | UroVys | LaVys |
| Controls | 15 | 4.02 | 4.59 | 20 | 5.07 | 4.55 |
| Stage IA | 13 | 15.56 | 11.00 | 15 | 13.04 | 16.19 |
| Stage IB | 7 | 17.53 | 16.74 | 7 | 23.78 | 16.53 |
| Stage | 8 | 23.32 | 17.97 | 7 | 12.95 | 13.13 |
| IIA-IIB | | | | | | |
| Stage | 9 | 11.67 | 7.79 | 10 | 20.55 | 12.37 |
| IIIA-IIIB | | | | | | |
| Stage IV | 9 | 31.75 | 43.60 | 18 | 23.82 | 32.95 |

**Tumor cell defined as having 2 or > chromosomal abnormalities

It is possible that lung cancer is initiated by a set of genes (3p22.1, 10q22.3) that are different from a set of genes that maintain tumor progression (LaVysion). To address this possibility, binary logistic regression models were run with individual stages and controls as binary outcome (Tables 10 and 11).

TABLE 12
Markers in univariate binary logistic regression model (Controls vs. stage 1A)

| Marker | B | S.E. | Wald | df | Sig. | OR | 0% C.I. Lo | 0% C.I. Up |
|---|---|---|---|---|---|---|---|---|
| cep3_Uro_gain | −1.45 | 0.64 | 5.22 | 1 | 0.022 | 0.23 | 0.07 | 0.81 |
| EGFR_Lav_gain | −1.67 | 0.74 | 5.09 | 1 | 0.024 | 0.19 | 0.04 | 0.80 |
| Cmyc_Lav_del | −2.51 | 1.12 | 4.98 | 1 | 0.026 | 0.08 | 0.01 | 0.74 |
| mono_cep_10 | −1.15 | 0.64 | 4.50 | 1 | 0.034 | 0.32 | 0.11 | 0.92 |
| del_10q | −0.81 | 0.39 | 4.24 | 1 | 0.039 | 0.44 | 0.21 | 0.96 |
| cep7_Uro_del | −2.27 | 1.14 | 3.97 | 1 | 0.046 | 0.10 | 0.01 | 0.96 |
| poly_cep_10 | −4.18 | 2.24 | 3.49 | 1 | 0.062 | 0.02 | 0.00 | 1.23 |
| cep17_Uro_gain | −2.08 | 1.17 | 3.17 | 1 | 0.075 | 0.12 | 0.01 | 1.24 |
| 9p21.3_Uro_gain | −2.56 | 1.51 | 3.07 | 1 | 0.080 | 0.07 | 0.00 | 1.37 |
| EGFR_Lav_del | −1.00 | 0.50 | 2.76 | 1 | 0.097 | 0.37 | 0.11 | 1.20 |
| del_3p | −0.24 | 0.16 | 2.15 | 1 | 0.143 | 0.79 | 0.57 | 1.08 |
| 5p15.2_Lav_del | −0.49 | 0.48 | 1.05 | 1 | 0.305 | 0.61 | 0.24 | 1.56 |
| mono_cep_3 | −0.63 | 0.80 | 0.62 | 1 | 0.432 | 0.53 | 0.11 | 2.57 |
| Cmyc_Lav_gain | −0.64 | 1.59 | 0.27 | 1 | 0.600 | 0.43 | 0.02 | 9.87 |
| poly_cep_3 | −0.19 | 0.56 | 0.11 | 1 | 0.735 | 0.83 | 0.28 | 2.48 |
| 5p15.2_Lav_gain | −0.25 | 0.74 | 0.11 | 1 | 0.738 | 0.78 | 0.18 | 3.34 |
| cep7_Uro_gain | −0.11 | 0.76 | 0.02 | 1 | 0.862 | 0.89 | 0.20 | 3.97 |
| 6p11q11_Lav_gain | 0.06 | 0.46 | 0.02 | 1 | 0.892 | 1.06 | 0.43 | 2.63 |
| 6p11q11_Lav_del | −0.02 | 0.15 | 0.01 | 1 | 0.917 | 0.98 | 0.74 | 1.31 |
| cep3_Uro_del | −0.09 | 1.17 | 0.01 | 1 | 0.937 | 0.91 | 0.09 | 9.08 |
| cep17_Uro_del | −0.01 | 0.15 | 0.01 | 1 | 0.940 | 0.99 | 0.73 | 1.34 |
| 9p21.3_Uro_del | 0.03 | 0.51 | 0.00 | 1 | 0.959 | 1.03 | 0.38 | 2.80 |

TABLE 13
Markers in univariate binary logistic regression model (Controls vs. stage IV)

| Marker | B | S.E. | Wald | df | Sig. | OR | 0% C.I. Lo | 0% C.I. Up |
|---|---|---|---|---|---|---|---|---|
| 9p21.3_Uro_del | −1.67 | 0.66 | 6.45 | 1 | 0.011 | 0.19 | 0.05 | 0.88 |
| 5p15.2_Lav_del | −1.18 | 0.48 | 6.01 | 1 | 0.014 | 0.31 | 0.12 | 0.79 |
| EGFR_Lav_gain | −1.70 | 0.72 | 5.58 | 1 | 0.018 | 0.18 | 0.04 | 0.75 |
| del_3p | −0.35 | 0.15 | 5.56 | 1 | 0.019 | 0.70 | 0.53 | 0.94 |
| cep3_Uro_gain | −1.78 | 0.76 | 5.44 | 1 | 0.020 | 0.17 | 0.04 | 0.75 |
| EGFR_Lav_del | −2.30 | 1.01 | 5.19 | 1 | 0.023 | 0.10 | 0.01 | 0.72 |
| del_10q | −1.06 | 0.51 | 4.28 | 1 | 0.038 | 0.35 | 0.13 | 0.95 |
| 9p21.3_Uro_gain | −2.97 | 1.55 | 3.66 | 1 | 0.056 | 0.05 | 0.00 | 1.07 |
| Cmyc_Lav_del | −1.75 | 0.94 | 3.49 | 1 | 0.062 | 0.17 | 0.03 | 1.09 |
| cep7_Uro_del | −1.60 | 0.88 | 3.33 | 1 | 0.068 | 0.20 | 0.04 | 1.12 |
| poly_cep_3 | −0.88 | 0.52 | 2.83 | 1 | 0.092 | 0.41 | 0.15 | 1.16 |
| cep17_Uro_gain | −2.04 | 1.24 | 2.72 | 1 | 0.099 | 0.13 | 0.01 | 1.47 |
| mono_cep_10 | −0.70 | 0.43 | 2.70 | 1 | 0.100 | 0.50 | 0.22 | 1.14 |
| mono_cep_3 | −1.45 | 0.90 | 2.62 | 1 | 0.105 | 0.23 | 0.04 | 1.36 |
| cep7_Uro_gain | −1.01 | 0.64 | 2.48 | 1 | 0.115 | 0.36 | 0.10 | 1.28 |
| 5p15.2_Lav_gain | −0.83 | 0.54 | 2.31 | 1 | 0.128 | 0.44 | 0.15 | 1.27 |
| cep3_Uro_del | −2.64 | 1.76 | 2.25 | 1 | 0.133 | 0.07 | 0.00 | 2.24 |
| 6p11q11_Lav_gain | −0.56 | 0.52 | 1.19 | 1 | 0.275 | 0.57 | 0.21 | 1.57 |
| Cmyc_Lav_gain | −1.22 | 1.19 | 1.06 | 1 | 0.303 | 0.29 | 0.03 | 3.01 |
| cep17_Uro_del | −0.13 | 0.14 | 0.80 | 1 | 0.372 | 0.88 | 0.66 | 1.17 |
| poly_cep_10 | −0.37 | 1.36 | 0.07 | 1 | 0.784 | 0.69 | 0.05 | 9.91 |
| 6p11q11_Lav_del | 0.01 | 0.12 | 0.01 | 1 | 0.925 | 1.01 | 0.81 | 1.27 |

When using the LaVysion probe set as well as deletions of 10/10q22.3 and 3/3p22.1, there was a progressive increase in CTC/μ from controls through low to high stages of non-small cell lung cancer with the highest numbers of CTCs expressing these panels of markers in stage 1V disease (Table 14). However, using the UroVysion probe set and polysomies of 10/10q there was an increase in abnormalities up to stages III and II respectively, and thereafter there was loss of expression of these markers. There was also a significant deletion of the gene for EGFR in high stage cases versus low stages.

The latter finding reflects the biology of the disease in that different genetic profiles are more commonly expressed early in disease, during tumor initiation, and at intermediate stages and are lost in higher stages and vice versa. This may be very important at a therapeutic level to treat the phenotype of the CTC and not the primary tumor.

TABLE 14
Number or CTC in Blood according to updated staging and marker profile

| | | CTC/ul | CTC/ul | CTC/ul | CTC/ul | CTC/ul | CTC/ul |
|---|---|---|---|---|---|---|---|
| Stage Code | | according to | according to | according to | according to | according to | according to |
| Revised 1/31/08 | | UroVysion | LaVysion | 3p | abn3p/cep3 | 10q | abn10q/cep10 |
| Controls | Mean | 4.02 | 4.20 | 20.64 | 1.06 | 9.62 | 1.90 |
| | Median | 1.82 | .00 | 12.41 | .00 | 6.65 | .00 |
| | Std. Deviation | 5.00 | 8.47 | 22.17 | 1.87 | 9.02 | 4.56 |
| | Minimum | .00 | .00 | .00 | .00 | .00 | .00 |
| | Maximum | 13.96 | 30.83 | 84.40 | 5.62 | 27.45 | 14.47 |
| | N | 15.00 | 14.00 | 16.00 | 16.00 | 16.00 | 16.00 |
| IA | Mean | 15.60 | 12.87 | 58.70 | 8.62 | 19.45 | 15.93 |
| | Median | 19.59 | 7.86 | 30.41 | 6.66 | 12.02 | 11.17 |
| | Std. Deviation | 13.29 | 11.46 | 48.38 | 8.61 | 15.15 | 21.32 |
| | Minimum | .00 | .00 | 3.44 | .00 | 1.21 | .00 |
| | Maximum | 34.75 | 33.99 | 154.76 | 26.30 | 48.63 | 79.63 |
| | N | 14.00 | 13.00 | 12.00 | 12.00 | 13.00 | 13.00 |
| IB | Mean | 13.47 | 18.19 | 32.91 | 10.93 | 23.21 | 6.86 |
| | Median | 11.49 | 14.80 | 25.11 | 6.44 | 19.47 | 8.00 |
| | Std. Deviation | 9.46 | 10.26 | 28.00 | 10.40 | 21.58 | 3.19 |
| | Minimum | .00 | 8.01 | 3.18 | .00 | 2.48 | 2.22 |
| | Maximum | 27.42 | 31.95 | 67.61 | 24.14 | 51.42 | 9.24 |
| | N | 6.00 | 6.00 | 5.00 | 5.00 | 4.00 | 4.00 |
| IIA-IIB | Mean | 22.94 | 20.11 | 40.13 | 15.30 | 26.33 | 19.85 |
| | Median | 10.65 | 15.87 | 54.13 | 12.56 | 24.40 | 18.13 |
| | Std. Deviation | 23.90 | 17.66 | 33.94 | 14.71 | 13.67 | 23.05 |
| | Minimum | 1.03 | 2.48 | 3.80 | .00 | 5.31 | .00 |
| | Maximum | 73.73 | 54.89 | 109.00 | 43.27 | 49.07 | 79.92 |
| | N | 10.00 | 10.00 | 10.00 | 10.00 | 10.00 | 10.00 |
| IIIA-IIIB | Mean | 24.75 | 19.18 | 22.59 | 6.40 | 37.11 | 5.90 |
| | Median | 8.29 | 11.13 | 5.02 | 3.81 | 5.72 | 3.52 |
| | Std. Deviation | 35.29 | 32.37 | 25.85 | 7.92 | 65.48 | 10.32 |
| | Minimum | 1.79 | .57 | 1.89 | .00 | 1.44 | .00 |
| | Maximum | 95.77 | 108.41 | 77.38 | 23.18 | 214.71 | 34.29 |
| | N | 10.00 | 10.00 | 9.00 | 9.00 | 10.00 | 10.00 |
| IV | Mean | 16.86 | 33.65 | 76.76 | 3.15 | 44.63 | 7.68 |
| | Median | 12.00 | 9.52 | 39.51 | .01 | 23.20 | 3.13 |
| | Std. Deviation | 25.14 | 82.82 | 116.57 | 6.24 | 58.15 | 14.40 |
| | Minimum | .04 | .00 | .00 | .00 | .13 | .00 |
| | Maximum | 109.94 | 340.58 | 492.88 | 25.10 | 231.31 | 52.60 |
| | N | 17.00 | 16.00 | 17.00 | 17.00 | 16.00 | 16.00 |
| Total | Mean | 15.60 | 10.36 | 44.76 | 6.37 | 26.80 | 9.30 |
| | Median | 9.34 | 8.44 | 29.32 | 2.19 | 15.81 | 3.25 |
| | Std. Deviation | 21.56 | 42.90 | 66.63 | 9.36 | 39.86 | 15.99 |
| | Minimum | .00 | .00 | .00 | .00 | .00 | .00 |
| | Maximum | 109.94 | 340.58 | 492.88 | 43.27 | 231.31 | 79.92 |
| | N | 72.00 | 69.00 | 69.00 | 69.00 | 69.00 | 69.00 |

It has also been shown that CTC's may be a more reliable way of estimating tumor burden ab initio than clinical staging, as demonstrated in the table below, where clinical staging was revised following surgery, and concurrently numbers of CTCs were shown to increase according to pathological stage. This is especially notable for stage IIA-B and stage IIIA IIIB (see highlighted areas in Table 15). For stage 1V, CTCs by LaVysion increase compared to the UroVysion panel due to most likely to biological expression of different markers from metastatic sites.

TABLE 15
Numbers of circulating tumor cells associated with clinical stage versus
pathological stage.

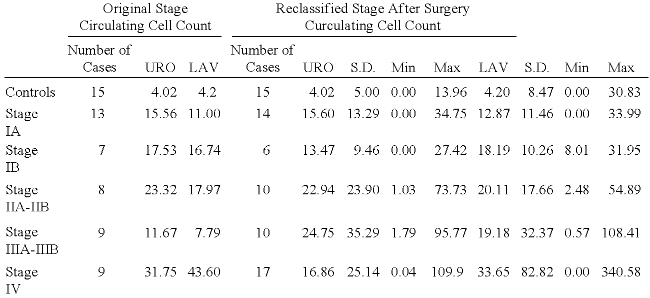

The finding for instance of higher numbers of CTCs than expected in a patient with low stage lung cancer, with a molecular phenotype more consistent with high stage disease, might be useful. For instance, it might place this patient more intensive surveillance regarding follow up (see Tables 10 and 11 (above) and Table 16, showing molecular staging before and after surgical resection).

TABLE 16
Molecular Staging by Circulating Tumor Cells*

| | Original Stage | | | | | | | | | | | |
|---|---|---|---|---|---|---|---|---|---|---|---|---|
| | Circulating | | | Reclasified Stage After Surgery | | | | | | | | |
| | Cell Count | | | Circulating Cell Count | | | | | | | | |
| | No. | Uro | LaV | No | Uro | S.D. | Min | Max | LaV | S.D. | Min | Max |
| Controls | 15 | 4.02 | 4.2 | 20 | 5.07 | 5.20 | 0.00 | 15.05 | 4.55 | 7.69 | 0.00 | 30.83 |
| Stage IA | 13 | 15.56 | 11.00 | 15 | 13.04 | 13.18 | 0.00 | 34.75 | 16.19 | 13.30 | 0.00 | 45.45 |
| Stage IB | 7 | 17.53 | 16.74 | 7 | 23.78 | 28.16 | 1.03 | 84.78 | 16.53 | 11.34 | 2.48 | 31.95 |
| IIA-IIB | 8 | 23.32 | 17.97 | 7 | 12.95 | 15.60 | 1.79 | 41.85 | 13.13 | 19.71 | 0.57 | 54.89 |
| IIIA-IIIB | 9 | 11.67 | 7.79 | 10 | 20.55 | 22.06 | 1.53 | 73.73 | 12.73 | 8.17 | 2.19 | 25.26 |
| Stage IV | 9 | 31.75 | 43.60 | 18 | 21.75 | 30.80 | 0.00 | 109.94 | 37.06 | 82.54 | 0.00 | 340.58 |

** Tumor cell defined as having 2 or > chromosomal abnormalities

### Example 3

### Correlation Between Blood and Corresponding Lung Cancer Tissue

It was also shown that there was a high correlation between biomarkers on CTCs compared to biomarkers from the primary lung tumors from the same subjects. See Table 17. This finding is important clinically as for example, tumors over- or under-expressing EGFR will have similar EGFR genetic abnormalities in the CTCs and can be used as a marker for anti-EGFR therapy.

TABLE 17
Correlations of circulating tumor cells in blood (CTC) and tumor washes
(TW)

| Correlations | p-value |
|---|---|
| Lavysion (EGFR, 5p15.2, C-myc, 6p11-q11) | 0.0002 |
| CTC correlated with TW EGFR Lav Gain | |
| Lavysion CTC (EGFR, 5p15.2, C-myc, 6p11-q11) | 0.001 |
| correlated with TW Cep7 Urov Gain | |
| Lavysion (EGFR, 5p15.2, C-myc, 6p11-q11) | 0.005 |
| CTC correlated with TW Single Gain Lav | |
| UroVysion (Cep3, Cep7 Cep17, 9p21.2) | 0.028 |
| CTC correlated with TW poly cep3/3p | |
| Lavysion (EGFR, 5p15.2, C-myc, 6p11-q11) | 0.029 |
| CTC correlated with TW mono cep3/3p | |
| Lavysion (EGFR, 5p15.2, C-myc, 6p11-q11) | 0.032 |
| CTC correlated with TW 5p15.2 Lav Del | |
| UroVysion (Cep3, Cep7 Cep17, 9p21.2) | 0.051 |
| CTC correlated with TW poly cep10/10q | |

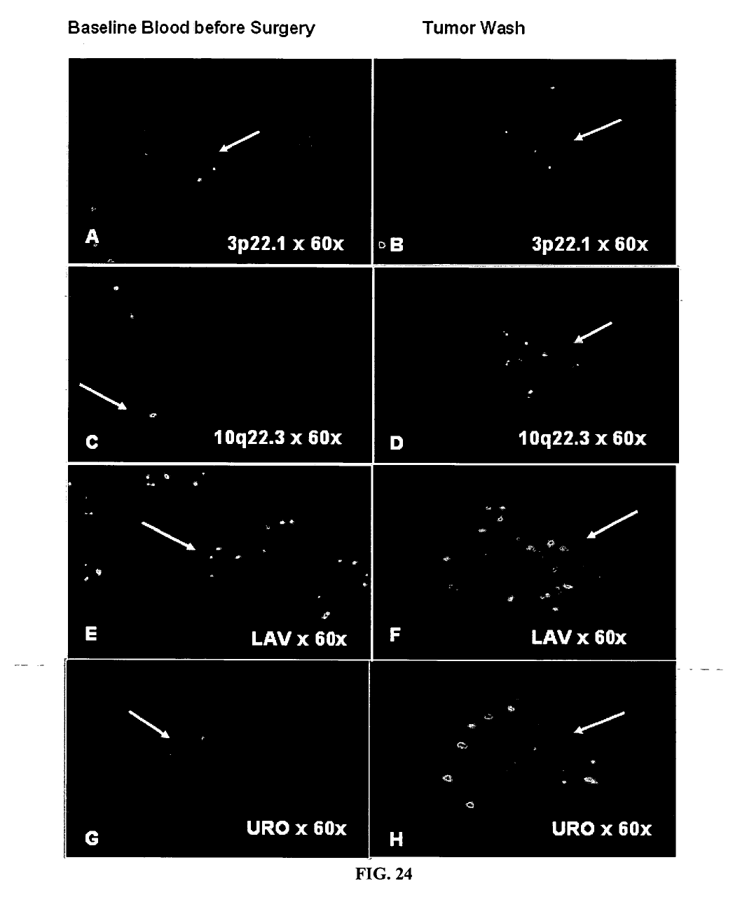

Paired sets of peripheral blood and tumor tissue were obtained from 21 patients who underwent surgical resection of their lung tumors. The same set of FISH probes was used in both the PBMCs and tumor specimens. A strong overall correlation between eight biomarker abnormalities in PMBCs and corresponding biomarkers in the tumor washes was observed; specifically, six were positively correlated and included gains of EGFR, C-Myc, 6p11-q11, 3p22.1 and different abnormalities in the URO set. See Table 18. EGFR gain in CACs was significantly correlated with EGFR gains in tumor washes for all disease stages, especially high stages (P≦0.01). Positively correlated chromosomal abnormalities were observed in the CTCs and those in the tumor cells by the URO probe set. In contrast, the genetic abnormalities in the LAV set in CTCs were negatively correlated with those in the tumor washes. An example of CTCs and corresponding tumor is shown (FIG. 24).

TABLE 18
Correlation between Chromosome Abnormalities as Measured in Blood versus Tumor

| | | | Spearman's Correlation (rho) between | | | |
|---|---|---|---|---|---|---|
| | | | blood and tumor wash measures | | | |
| | | Tumor | | | Stage I/ | Stage |
| % Chromosomal | Blood | Wash | | | II | III/IV |
| Abnormalities | Mean ± SD | Mean ± SD | N pairs | Overall | (N = 17) | (N = 4) |
| Gain | 1.03 ± 0.79 | 0.93 ± 1.43 | 20 | 0.416* | | |
| 3p22.1/CEP3 | | | | | | |
| EGFR gain | 2.89 ± 1.84 | 4.53 ± 5.20 | 18 | 0.602*** | 0.481* | 1.00*** |
| C-myc gain | 0.25 ± 0.59 | 1.09 ± 3.74 | 18 | 0.481** | | |
| 6p11-q11gain | 0.83 ± 1.16 | 1.69 ± 1.44 | 18 | 0.521** | 0.528* | −0.949* |
| Abn LAV | 1.74 ± 1.16 | 33.92 ± 26.56 | 18 | −0.416* | | |
| 9p21.3 gain | 0.57 ± 0.99 | 0.66 ± 1.05 | 21 | −0.445** | | |
| abn URO | 1.85 ± 1.03 | 30.63 ± 24.52 | 21 | 0.580** | 0.624*** | |
| URO CTC vs. | 19.23 ± 17.85 | 30.63 ± 24.52 | 20 | 0.579** | 0.617*** | |
| TW % abn | | | | | | |
| URO | | | | | | |
| LaV CTC vs | 16.13 ± 13.23 | 33.92 ± 26.56 | 17 | −0.302 | −0.600** | |
| TW | | | | | | |
| % abn LAV | | | | | | |

*= P < 0.10;
**= P < 0.05;
***= P < 0.01
Abbreviations:
CTC = Circulating Tumor Cells;
Abn = abnormality;
LAV = LAVysion;
URO = UroVysion;
TW = Tumor Wash

Eight of the DNA biomarkers in the PBMCs were significantly correlated with the resected lung tumors. The percentages of genomically altered cells for all 12 biomarkers tested correlated with the stage of NSCLC, with the lowest levels detected in patients with stage I disease and the highest detected in patients with stage III and IV disease. Further, the controls had significantly fewer genetically abnormal cells for all the biomarkers.

Certain chromosomal abnormalities were present in patients with early and late-stage NSCLC and were correlated with relapse and poor survival. Cytogenetically abnormal cells may have both deletions and gains of EGFR; however, only EGFR deletions correlated with relapse and poor survival. In view of the significant association of EGFR gain in CACs with those in primary tumors, blood may be used as a valuable non-invasive surrogate biomarker for EGFR overexpression. Other biomarkers were represented at lower levels in CTCs than in primary tumor cells suggesting that cells that enter the bloodstream from a tumor may have a genotype considerably different from that of the primary site. In general, CTCs in the study had fewer chromosomes and less genetic material than did primary tumors cells.

### Example 4

### Biomarker Abnormalities Associated with Disease Prediction

In Table 8 (above), some variables highlighted were significantly different among the cases and controls. Further evaluation of the role that these variables played in the risk of lung cancer was studied. However, the correlations among the outcomes within each abnormality group were high (and statistically significant). Therefore, the following variables were chosen from each group for further analyses. Note that these variables are representative of the other outcomes in their abnormality group and there was a statistically significant difference between cases and controls for these variable.

For each variable, a dichotomy was constructed using the 75^{th} percentile of the controls. Each dichotomized variable was then fit in a logistic regression and the odds ratio (OR) and 95% CI for each dichotomy adjusted by age and sex was calculated (Table 19).

TABLE 19
Distributions and risk estimates of lung cancer for selected CTCs

| | | Case | Control | | |
|---|---|---|---|---|---|
| | | patients | subjects | | |
| Marker | | N (%) | N (%) | OR (95% CI)* | PPV |
| 3p combined | <4.36 | 14 (24.1) | 19 (76.0) | 25.95 (4.95-136.06) | 93.1 |
| | ≧4.36 | 44 (75.9) | 6 (24.0) | | |
| 10q combined | <2.64 | 9 (15.3) | 19 (76.0) | 25.25 (6.06-105.33) | 89.8 |
| | ≧2.64 | 50 (84.7) | 6 (24.0) | | |
| LaVysion % Abnormalities | <0.90 | 13 (22.4) | 18 (75.0) | 9.78 (2.79-34.37) | 91.4 |
| | ≧0.90 | 45 (77.6) | 7 (25.0) | | |
| UroVysion % Abnormalities | <1.25 | 18 (30.5) | 19 (76.0) | 6.35 (1.97-20.54) | 91.5 |
| | ≧1.25 | 41 (69.5) | 6 (24.0) | | |
| CTC UroV + LaV | <1.98 | 10 (17.2) | 19 (76.0) | 16.12 (4.26-60.99) | 93.1 |
| | ≧1.98 | 48 (82.8) | 6 (24.0) | | |
| CTC 3p | <27.0 | 27 (46.6) | 19 (76.0) | 8.92 (2.1-37.89) | 89.7 |
| | ≧27.03 | 31 (53.4) | 6 (24.0) | | |
| CTC Abnormal 3p | <1.84 | 26 (44.8) | 19 (76.0) | 4.70 (1.37-16.10) | 93.1 |
| | ≧1.84 | 32 (55.2) | 6 (24.0) | | |
| CTC 10q | <9.69 | 18 (30.5) | 19 (76.0) | 12.00 (3.01-47.90) | 89.8 |
| | ≧9.69 | 41 (69.5) | 6 (24.0) | | |
| CTC Abnormal 10q | <4.78 | 28 (47.5) | 19 (76.0) | 3.30 (1.03-10.61) | 98.3 |
| | ≧4.78 | 31 (52.5) | 6 (24.0) | | |

*Adjusted by age and sex;
Dichotomy for all variables at 75^{th} percentile of controls;
PPV = Positive predictive value.

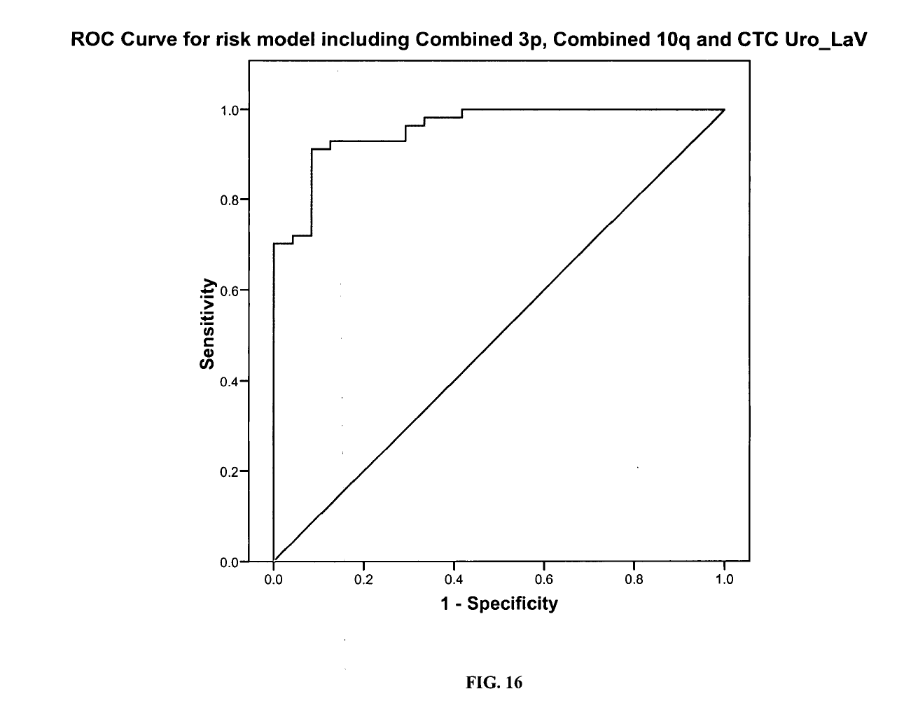

To obtain the final risk model, all of the above dichotomized variables were included in a forward logistic regression. The final model and risk estimates are given below. This final model has an AUC of 95.8% as displayed in FIG. 16.

TABLE 20
Final risk model

| | OR (95% CI)* | AUC |
|---|---|---|
| 3p combined | 6.18 (0.97-48.36) | 95.8% |
| 10q combined | 13.72 (2.32-81.20) | |
| CTC UroV + LaV | 7.68 (1.40-41.01) | |

*Adjusted by age and sex;
AUC = Area under the ROC curve.

The risk model in Table 20 can be used to calculate the probability of developing lung cancer based on a risk profile.

Profile 1: Male participant, 60 years of age with low combined 3p, low combined 10q and low CTC (measured by UroV+LaV). This individual has a 7% chance of developing lung cancer.

Profile 2: In contrast, another male participant of the same age with high combined 3p, high combined 10q and high CTC (measured by UroV+LaV) would have an 80% chance of developing lung cancer.

This shows that individuals who have high values for the biomarkers are at higher risk for lung cancer.

### Example 5

### Biomarker Abnormalities Associated with Disease Recurrence

Thirty-four NSCLC patients experienced no relapse and twenty-two patients experienced persistent or relapsed disease. Using Levene's t test for equality of variances, it was shown that combined deletions 3p and 10q were the most significant biomarkers (p<0.0002) for relapse or persistent disease, followed by deletions of 3p. Other significant biomarkers that were significantly correlated with relapse or persistent disease included abnormalities of 3p/cep3, deletion 10, deletions and polysomies of cep3/3p22.1 and cep 10/10q, gain of cep7, a single gain of any probe in the LaVysion and Urovysion set, cep 17, LAV+URO abnormal cells, and CTCs/μl according to 10q deletion or cep3/abnormality 3p22.1 (see Tables 21, 22, 25, and 26).

TABLE 21
T-test comparing relapse versus no relapse; means of CTCs for
relapse/persistent disease patients (24) versus no relapse (32)
Group Statistics

| | No | | | | |
|---|---|---|---|---|---|
| | Relapse/Persistent | | | | |
| | Disease/Relapse | | | Std. | |
| | Code | N | Mean | Deviation | Std. Error Mean |
| del 3p | No Relapse | 32 | 4.287 | 2.946 | 0.521 |
| | Relapse | 24 | 8.348 | 4.879 | 0.996 |
| abn 3p/cep3 | No Relapse | 32 | 1.132 | 1.137 | 0.201 |
| | Relapse | 24 | 0.519 | 0.585 | 0.119 |
| mono cep 3/3p | No Relapse | 32 | 0.685 | 0.782 | 0.138 |
| | Relapse | 24 | 0.868 | 0.579 | 0.118 |
| poly cep 3/3p | No Relapse | 32 | 1.249 | 1.428 | 0.252 |
| | Relapse | 24 | 1.461 | 1.200 | 0.245 |
| del 10q | No Relapse | 34 | 2.843 | 2.501 | 0.429 |
| | Relapse | 22 | 6.505 | 5.198 | 1.108 |
| abn 10q/cep 10 | No Relapse | 34 | 1.425 | 1.426 | 0.244 |
| | Relapse | 22 | 1.121 | 1.573 | 0.335 |
| mono cep 10/10q | No Relapse | 34 | 1.206 | 1.252 | 0.215 |
| | Relapse | 22 | 1.606 | 1.361 | 0.290 |
| poly cep 10/10q | No Relapse | 34 | 0.584 | 0.571 | 0.098 |
| | Relapse | 22 | 0.582 | 0.850 | 0.181 |
| del 3p + del 10q | No Relapse | 31 | 7.425 | 4.041 | 0.726 |
| | Relapse | 22 | 15.135 | 7.711 | 1.644 |
| del + abn + mono + | No Relapse | 32 | 7.353 | 3.526 | 0.623 |
| poly 3p | Relapse | 24 | 11.196 | 5.729 | 1.169 |
| del + abn + mono + | No Relapse | 34 | 6.058 | 3.441 | 0.590 |
| poly 10q | Relapse | 22 | 9.814 | 5.912 | 1.260 |
| single del Lav | No Relapse | 35 | 7.442 | 4.075 | 0.689 |
| | Relapse | 23 | 8.290 | 6.596 | 1.375 |
| EGFR Lav del | No Relapse | 35 | 1.333 | 1.421 | 0.240 |
| | Relapse | 23 | 1.984 | 1.441 | 0.300 |
| 5p15.2 Lav del | No Relapse | 35 | 1.764 | 2.375 | 0.401 |
| | Relapse | 23 | 2.343 | 2.493 | 0.520 |
| C-myc Lav del | No Relapse | 35 | 0.549 | 0.677 | 0.114 |
| | Relapse | 23 | 0.919 | 1.061 | 0.221 |
| 6p11-q11 Lav del | No Relapse | 35 | 3.811 | 2.362 | 0.399 |
| | Relapse | 23 | 3.000 | 4.132 | 0.862 |
| single gain Lav | No Relapse | 35 | 4.649 | 2.610 | 0.441 |
| | Relapse | 22 | 7.214 | 4.319 | 0.921 |
| EGFR Lav gain | No Relapse | 35 | 3.201 | 2.438 | 0.412 |
| | Relapse | 23 | 4.465 | 2.964 | 0.618 |
| 5p15.2 Lav gain | No Relapse | 35 | 0.519 | 0.755 | 0.128 |
| | Relapse | 23 | 0.642 | 1.063 | 0.222 |
| C-myc Lav gain | No Relapse | 35 | 0.162 | 0.461 | 0.078 |
| | Relapse | 23 | 0.374 | 0.626 | 0.131 |
| 6p11-q11 Lav gain | No Relapse | 35 | 0.819 | 1.177 | 0.199 |
| | Relapse | 23 | 1.402 | 0.962 | 0.201 |
| % abn Lav | No Relapse | 35 | 1.811 | 1.280 | 0.216 |
| | Relapse | 23 | 3.144 | 2.979 | 0.621 |
| single del Uro | No Relapse | 36 | 8.679 | 3.423 | 0.571 |
| | Relapse | 24 | 9.058 | 3.219 | 0.657 |
| cep3 Uro del | No Relapse | 36 | 0.246 | 0.566 | 0.094 |
| | Relapse | 24 | 0.325 | 0.449 | 0.092 |
| cep7 Uro del | No Relapse | 36 | 1.322 | 1.997 | 0.333 |
| | Relapse | 24 | 1.152 | 1.565 | 0.320 |
| 9p21.3 Uro del | No Relapse | 36 | 1.268 | 1.862 | 0.310 |
| | Relapse | 24 | 2.301 | 1.483 | 0.303 |
| cep17 Uro del | No Relapse | 36 | 5.821 | 2.744 | 0.457 |
| | Relapse | 24 | 5.483 | 2.102 | 0.429 |
| single gain Uro | No Relapse | 36 | 4.488 | 3.097 | 0.516 |
| | Relapse | 24 | 6.565 | 3.869 | 0.790 |
| cep3 Uro gain | No Relapse | 36 | 3.139 | 2.568 | 0.428 |
| | Relapse | 24 | 4.041 | 3.419 | 0.698 |
| cep7 Uro gain | No Relapse | 35 | 0.298 | 0.417 | 0.070 |
| | Relapse | 24 | 0.883 | 0.883 | 0.180 |
| 9p21.3 Uro gain | No Relapse | 36 | 0.529 | 0.701 | 0.117 |
| | Relapse | 24 | 0.725 | 0.674 | 0.138 |
| cep17 Uro gain | No Relapse | 36 | 0.473 | 0.693 | 0.116 |
| | Relapse | 24 | 1.018 | 1.339 | 0.273 |
| % abn Uro | No Relapse | 36 | 1.886 | 1.710 | 0.285 |
| | Relapse | 24 | 2.476 | 1.314 | 0.268 |
| CTC/ul according to | No Relapse | 35 | 16.681 | 20.002 | 3.381 |
| UroVysion | Relapse | 23 | 21.712 | 27.101 | 5.651 |
| CTC/ul according to | No Relapse | 34 | 15.313 | 13.546 | 2.323 |
| LaVysion | Relapse | 22 | 31.792 | 72.734 | 15.507 |
| Lav + UroV Abnormal | No Relapse | 35 | 3.675 | 2.236 | 0.378 |
| Cells | Relapse | 23 | 5.641 | 3.827 | 0.798 |
| CTC/ul according to 3p | No Relapse | 31 | 39.617 | 37.621 | 6.757 |
| | Relapse | 23 | 71.095 | 102.160 | 21.302 |
| CTC/ul according to | No Relapse | 31 | 10.512 | 11.177 | 2.008 |
| abn3p/cep3 | Relapse | 23 | 4.220 | 7.110 | 1.483 |
| CTC/ul according to | No Relapse | 33 | 20.466 | 16.228 | 2.825 |
| 10q | Relapse | 21 | 54.151 | 65.136 | 14.214 |
| CTC/ul according to | No Relapse | 33 | 11.480 | 14.954 | 2.603 |
| abn10q/cep10 | Relapse | 21 | 11.273 | 21.096 | 4.604 |

TABLE 22
Factors associated with relapse or persistent disease; independent samples
test
Independent Samples Test

| | t-test for Equality of Means | | |
|---|---|---|---|
| | | 95% Confidence | |
| | | Interval of | |
| | | the Difference | |
| | Sig (2-tailed) | Lower | Upper |
| CTC/ul according to 10q | 0.030 | −63.772 | −3.597 |
| del 3p + del 10q | 0.0002 | −11.385 | −4.036 |
| del 10q | 0.005 | −6.099 | −1.225 |
| cep7 Uro gain | 0.005 | −0.981 | −0.190 |
| Lav + UroV Abnormal Cells | 0.033 | −3.765 | −0.167 |
| del + abn + mono + poly 3p | 0.006 | −6.531 | −1.154 |
| CTC/ul according to abn3p/cep3 | 0.015 | 1.283 | 11.303 |
| abn 3p/cep3 | 0.012 | 0.143 | 1.083 |
| del 3p | 0.001 | −6.341 | −1.779 |
| del + abn + mono + poly 10q | 0.011 | −6.597 | −0.915 |
| single gain Lav | 0.007 | −4.401 | −0.730 |
| single gain Uro | 0.025 | −3.884 | −0.272 |
| 9p21.3 Uro del | 0.027 | −1.941 | −0.125 |

RECURRENCE: The variables within the same abnormality group were highly correlated, therefore, a representative (most significant) abnormality was chosen from each group for further analyses, namely 3p deletions, 10q deletions, UroVysion 9p21.3 Deletions, UroVysion Cep7 gains, and Uro+Lav % Abnormaltities. The stage was recorded as follows (IA=1, IB=2, IIAB=3, IIIAB=4 and IV=5) and regressed stage onto the previously listed predictor variables. This model resulted in an R-squared of 55.9% (P=0.002).

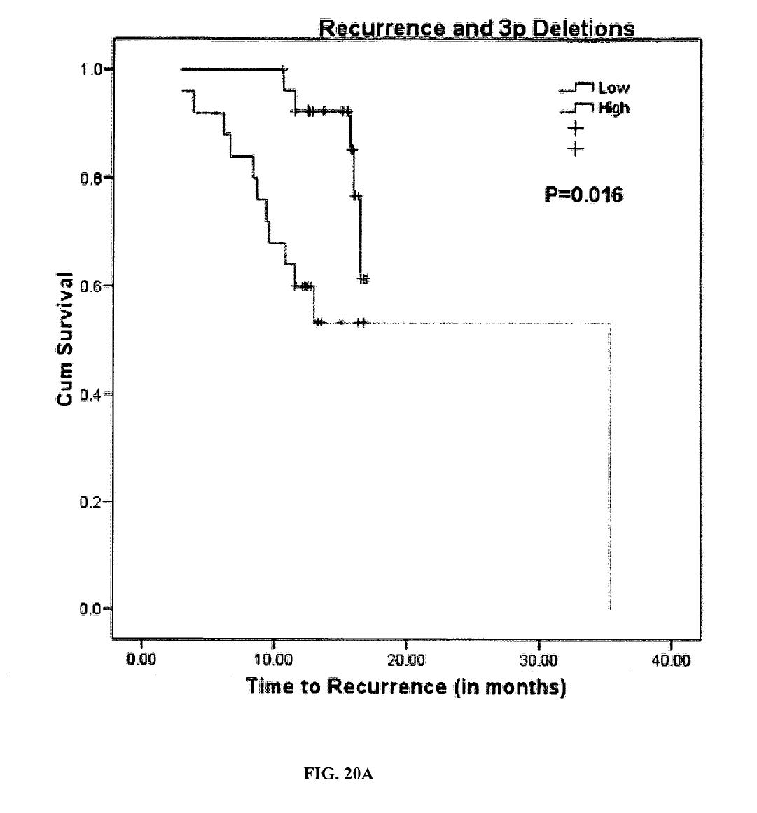

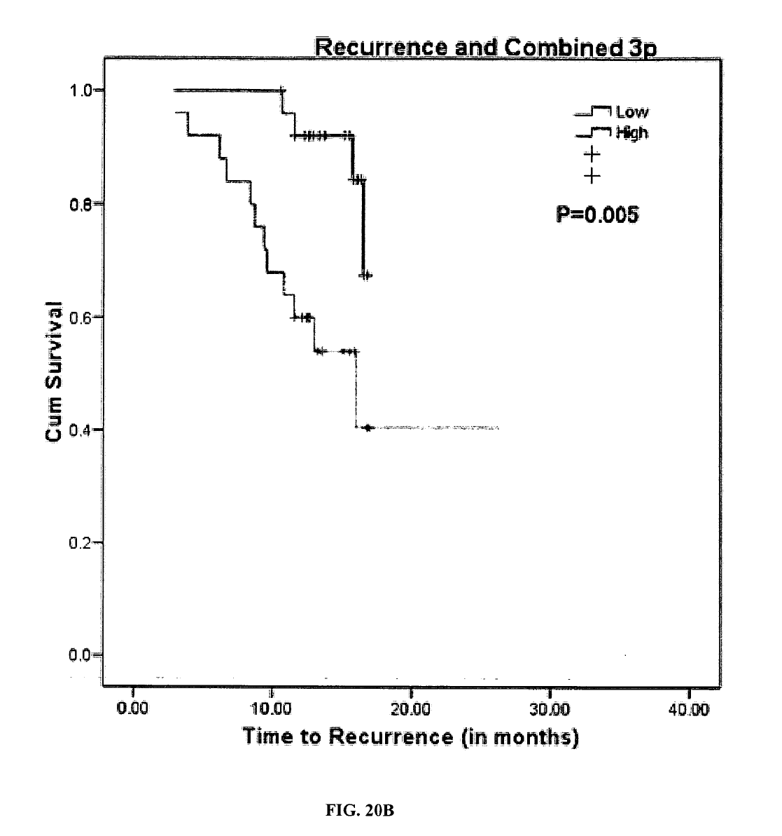

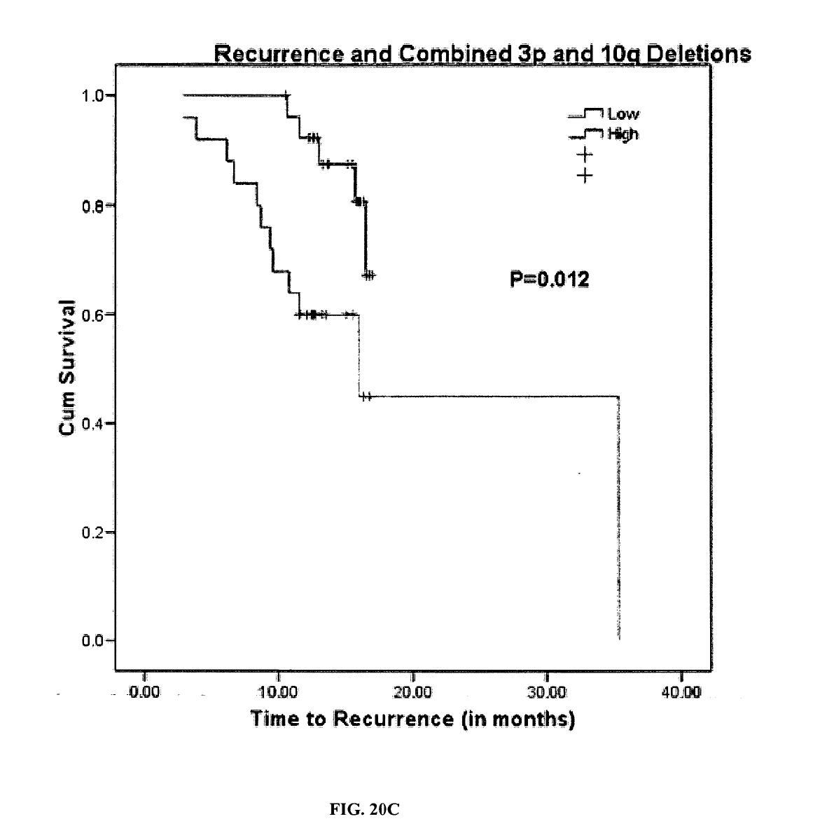

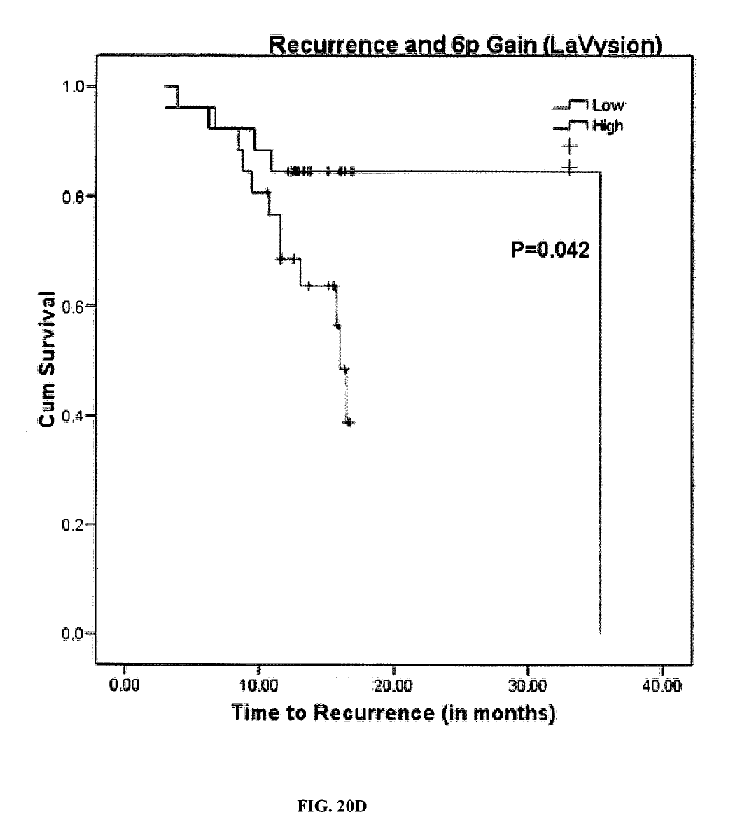

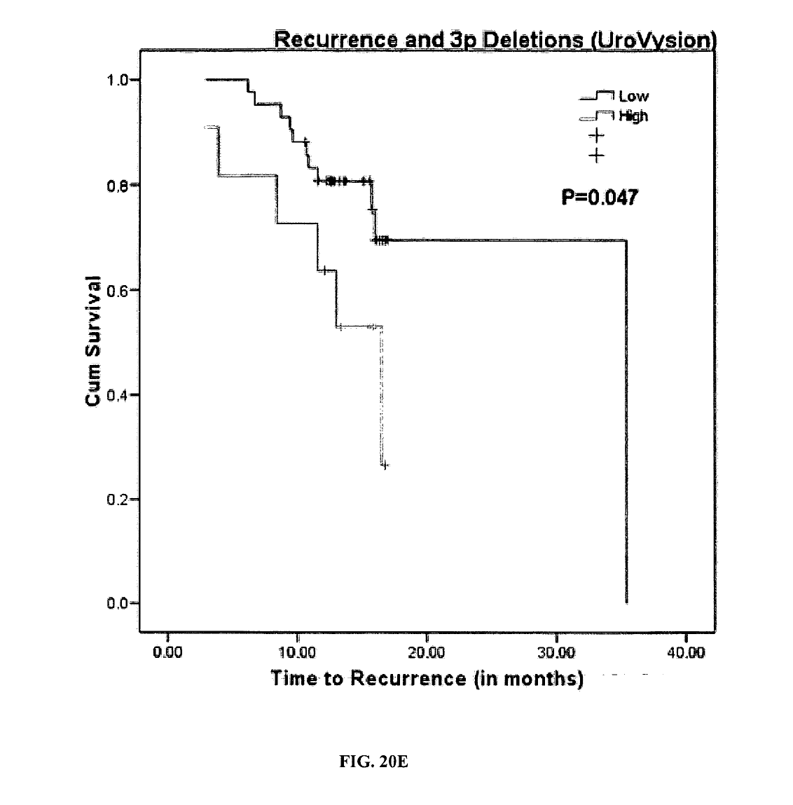

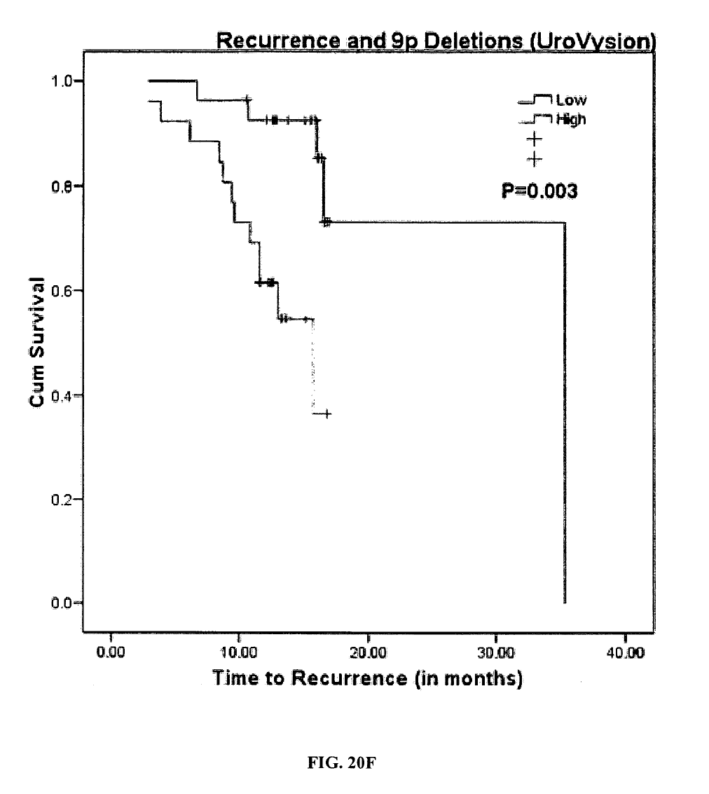

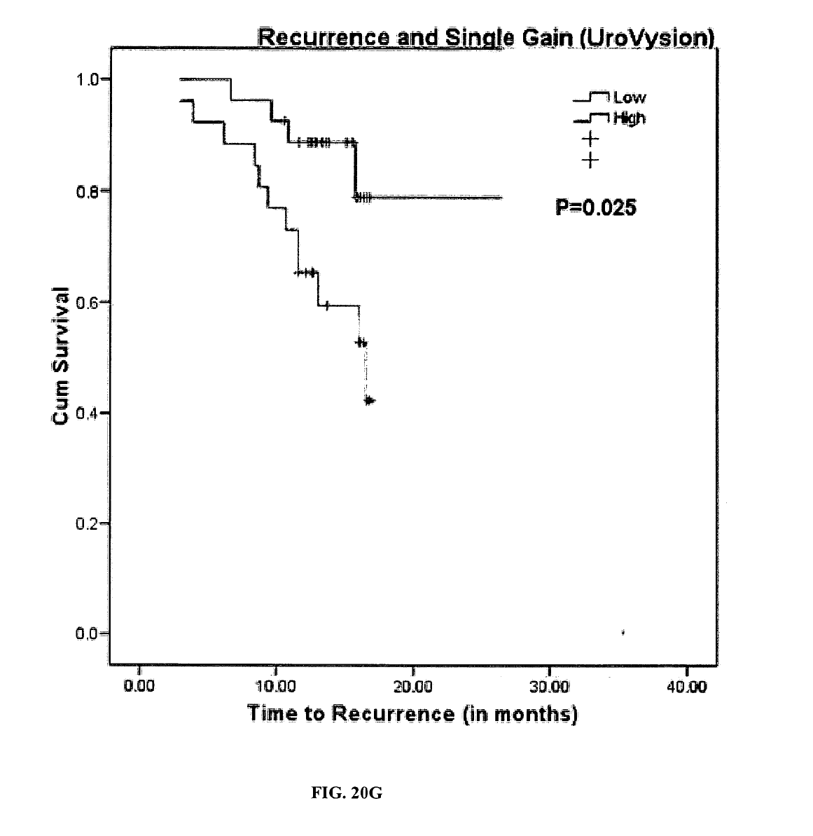

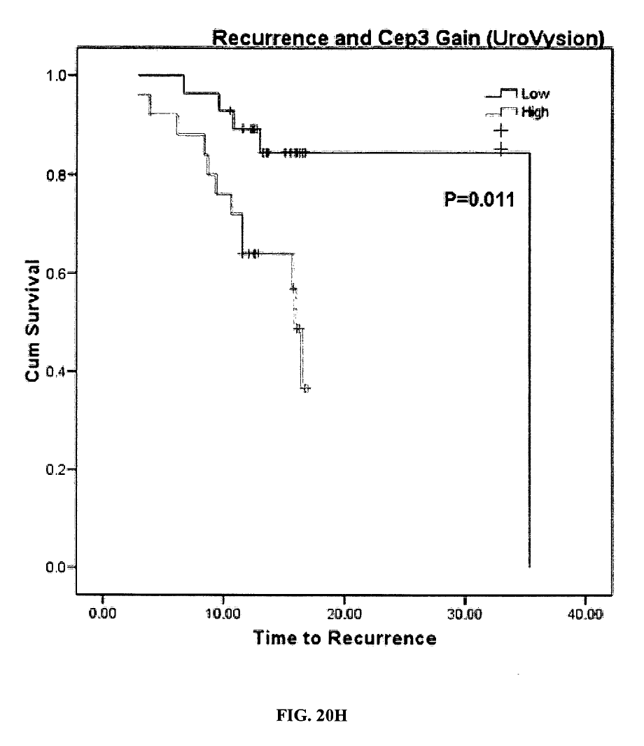

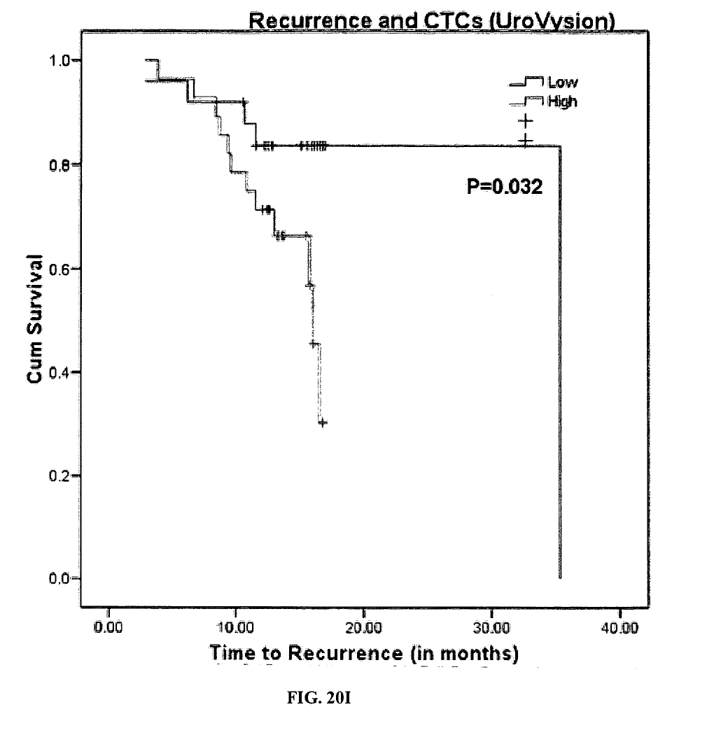

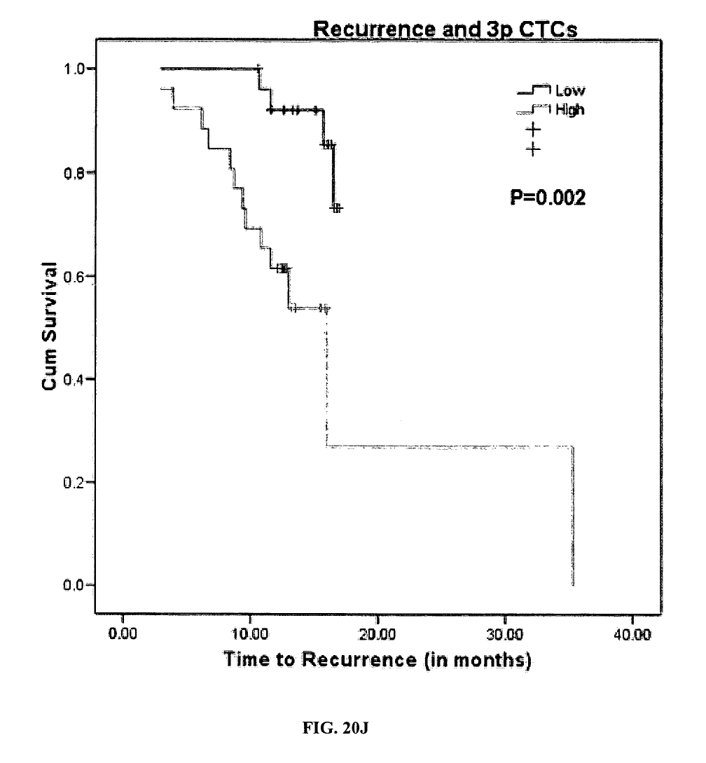

To evaluate the role of the biomarkers in recurrence, each marker was dichotomized at the median value (for all cases). Each dichotomy was evaluated using the Kaplan-Meier method (Table 23)(FIGS. 20A-J). The following markers are significantly associated for recurrence at the P=0.10 level: 10q Mono, Combined 10q, 6p Deletions (LaVysion), 5p Gain (LaVysion), % Abnormalities (LaVysion), Cep7 Gain (UroVysion).

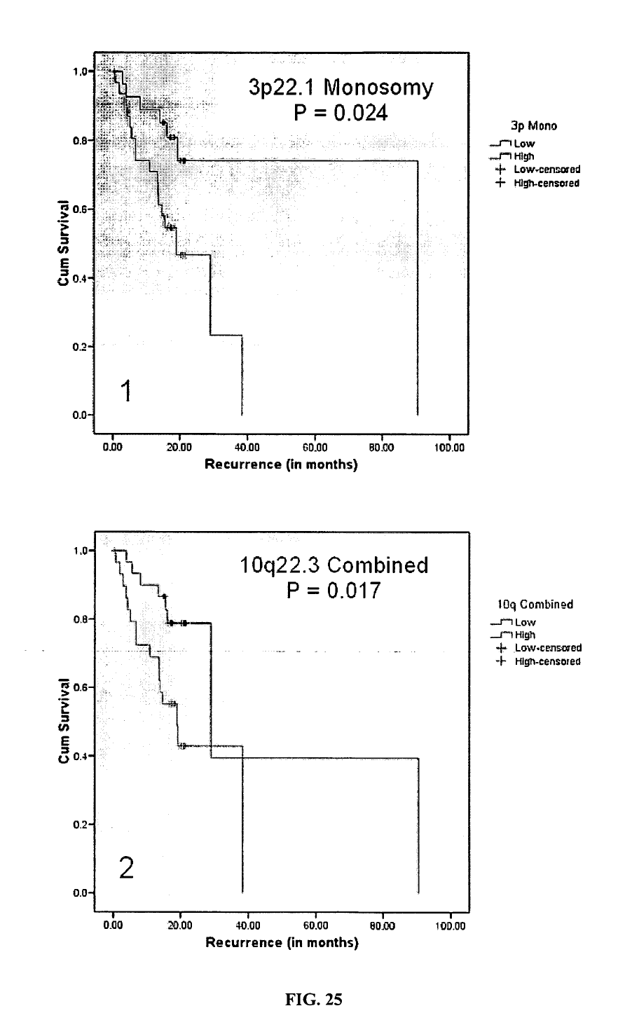

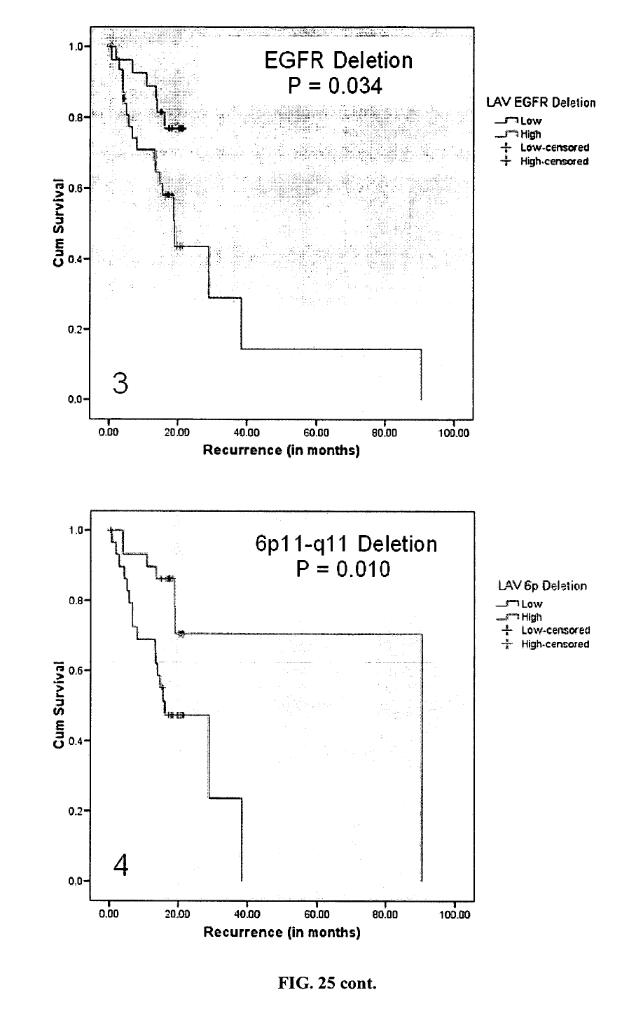

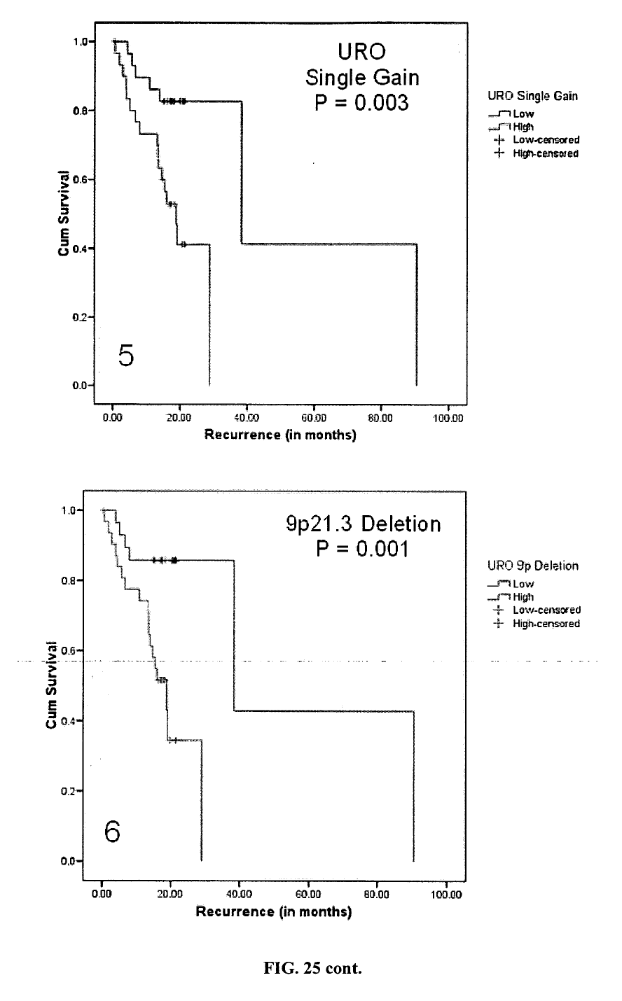

Twenty-three (39%) patients had disease recurrence. The median time to recurrence was 29 months [95% confidence interval (CI), 15.49 to 42.51 months]. Twenty biomarkers were significant at the 10% level, of which, twelve were significant at 5% level in Kaplan-Meier analyses for disease recurrence (FIG. 25A and Table 23). Of these, three were significant at the univariate level using the Cox proportional hazards model: namely 5p15.2 gain, 3p22.1 deletion, and a single URO gain. However, these biomarkers were not significant for disease recurrence after adjustment for age, sex, and disease stage.

TABLE 23
Markers associated with disease recurrence

| | | Median Survival (in months) | | | Cox Model | Cox Model |
|---|---|---|---|---|---|---|
| | | 95% CI | | | Unadj HR | Adjusted HR^{b} |
| Marker | | Low | High | P^{a} | (95% CI) | (95% CI) |
| 3p22.1 | Deletions | — | 18.8 (14.2-23.5) | 0.071 | — | — |
| | Monosomy | 90.5 (—) | 18.9 (10.8-26.9) | 0.024 | — | — |
| | Combined Abnormalities | — | 18.9 (9.5-28.2) | 0.030 | — | — |
| 3p/10q | Deletions | 29.0 (—) | 15.5 (9.5-21.5) | 0.019 | — | — |
| 10q.22.3 | Monosomy | 90.5 (—) | 16.0 (9.7-22.3) | 0.066 | — | — |
| | Combined Abnormalities | 29.0 (10.7-47.3) | 18.9 (9.9-27.8) | 0.017 | — | — |
| LaVysion | EGFR del | — | 18.9 (10.2-27.6) | 0.034 | — | — |
| | 6p del | 15.5 (12.3-18.7) | 90.4 (—) | 0.010 | — | — |
| | Single gain | 90.4 (—) | 18.7 (14.0-23.7) | 0.072 | — | — |
| | 5p gain | 29.0 (10.0-48.0) | 16.0 (9.5-22.5) | 0.046 | 3.04 (1.15-8.01) | 1.57 (0.55-4.51) |
| | 6p gain | 19.2 (7.6-30.8) | 16.0 (8.9-23.1) | 0.039 | — | — |
| | Abnormal | 29.0 (14.8-43.2) | 18.9 (10.9-26.8) | 0.085 | — | — |
| UroVysion | CEP3 | 38.4 (—) | 16.0 (0.0-34.1) | 0.072 | 4.03 (1.39-11.64) | 1.87 (0.64-5.51) |
| | Deletions | 37.4 (0.0-80.8) | 15.5 (12.4-18.6) | 0.001 | — | — |
| | 9p Deletions | 38.4 (4.2-72.6) | 16.0 (10.1-22.0) | 0.003 | 3.24 (1.13-9.26) | 2.14 (0.67-6.79) |
| | Single Gain | 38.4 (19.9-56.8) | 16.0 (11.7-20.4) | 0.004 | — | — |
| | CEP3 Gain | 29.0 (11.5-46.5) | 18.9 (9.8-28.0) | 0.028 | — | — |
| | Abnormal | | | | | |
| Circulating | UroVysion | 38.4 (10.6-66.1) | 18.9 (7.8-30.0) | 0.062 | — | — |
| Tumor Cells | Uro/Lav | 29.0 (11.0-47.0) | 18.9 (10.3-27.4) | 0.051 | — | — |
| | 3p22.1 | — | 19.2 (7.1-31.3) | 0.095 | — | — |

P-value from Kaplan-Meier log-rank test,
^{b}HR adjusted by age, sex and stage;
— Median survival estimates not calculable

### Example 7

### Biomarker Abnormalities Associated with Survival

Again, the variables within the same abnormality group were highly correlated, therefore, a representative (most significant) abnormality was chosen from each group for further analyses, namely 3p deletions, 10q deletions, UroVysion 9p21.3 Deletions, UroVysion Cep7 gains, and Uro+Lav % Abnormaltities. The stage was recorded as follows (IA=1, IB=2, IIAB=3, IIIAB=4 and IV=5) and regressed stage onto the previously listed predictor variables. This model resulted in an R-squared of 55.9% (P=0.002).

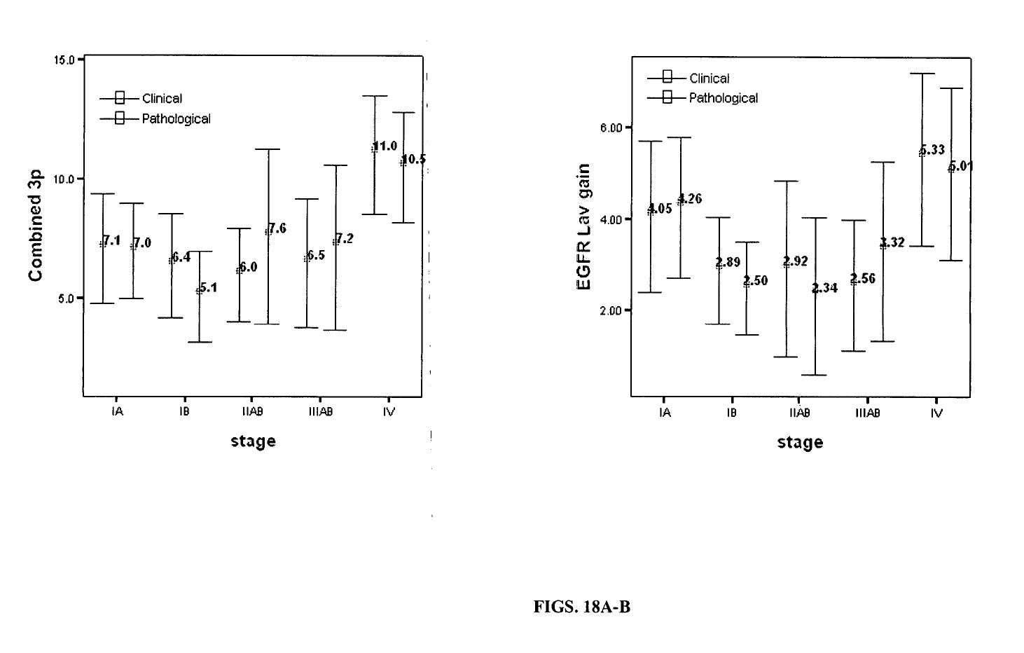

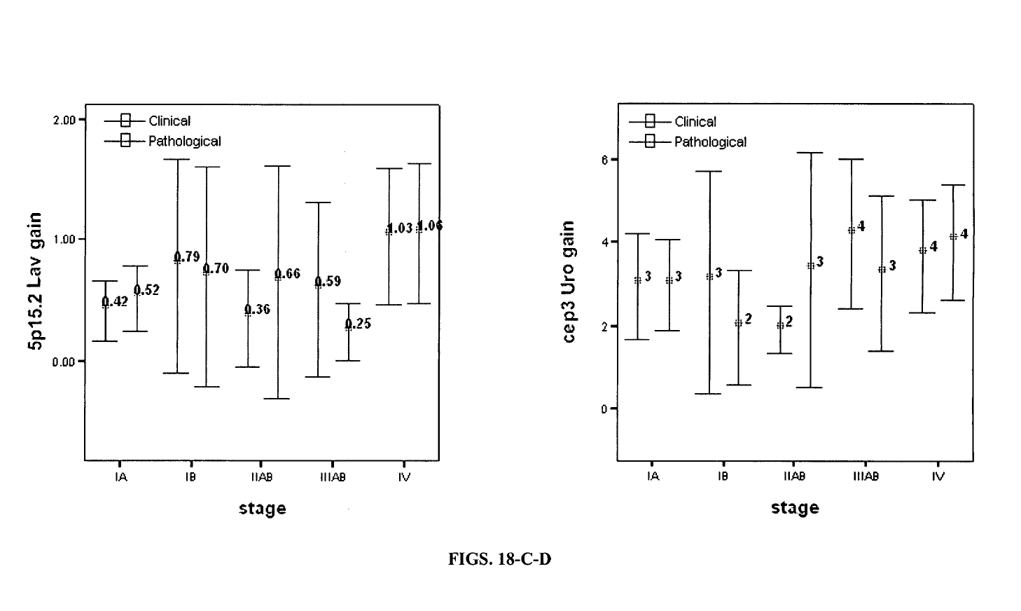

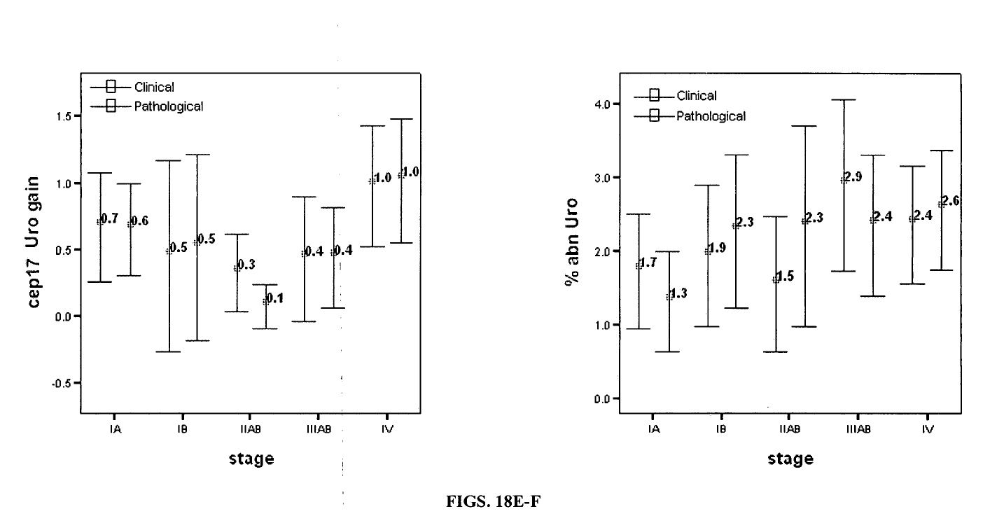

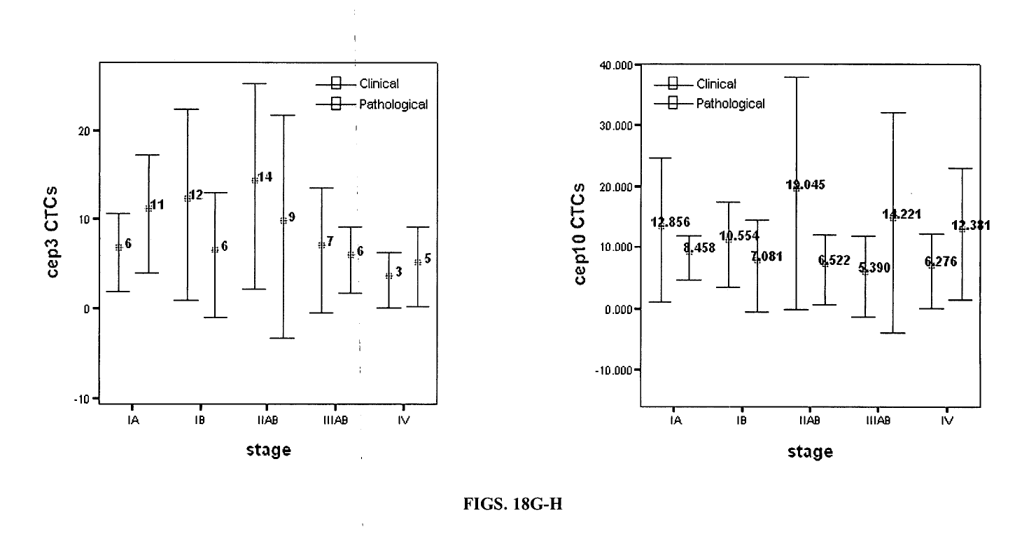

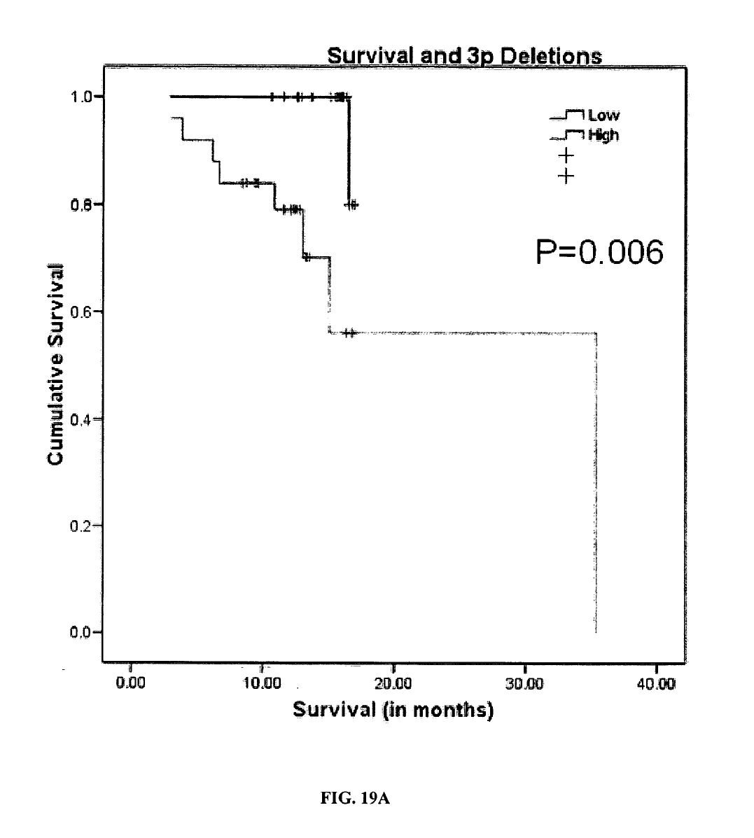

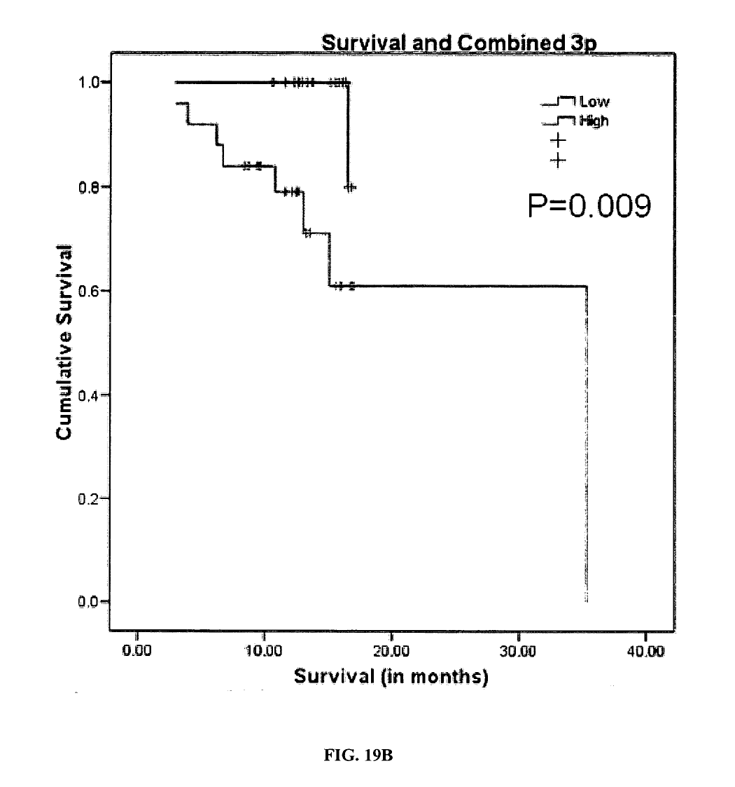

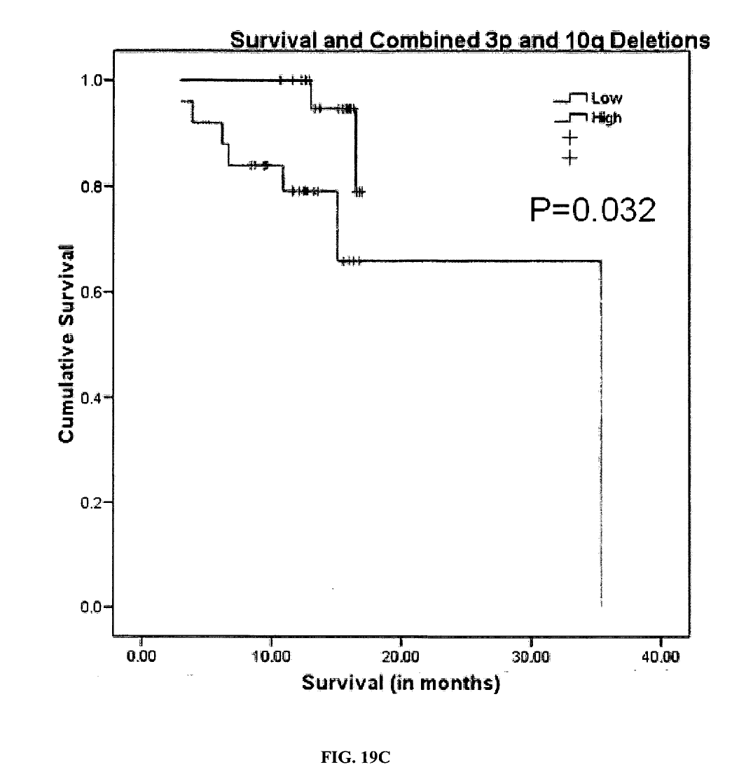

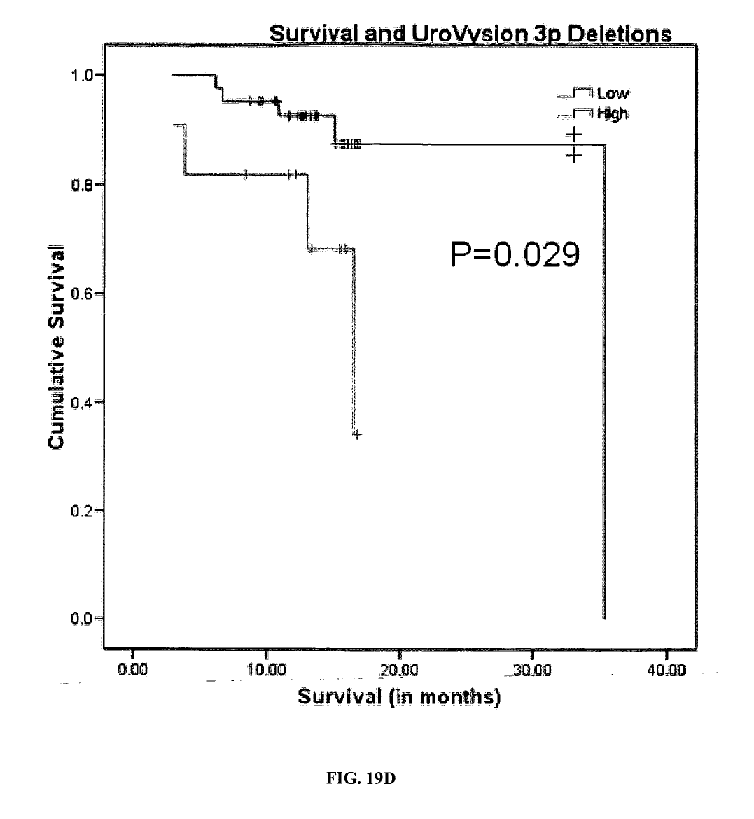

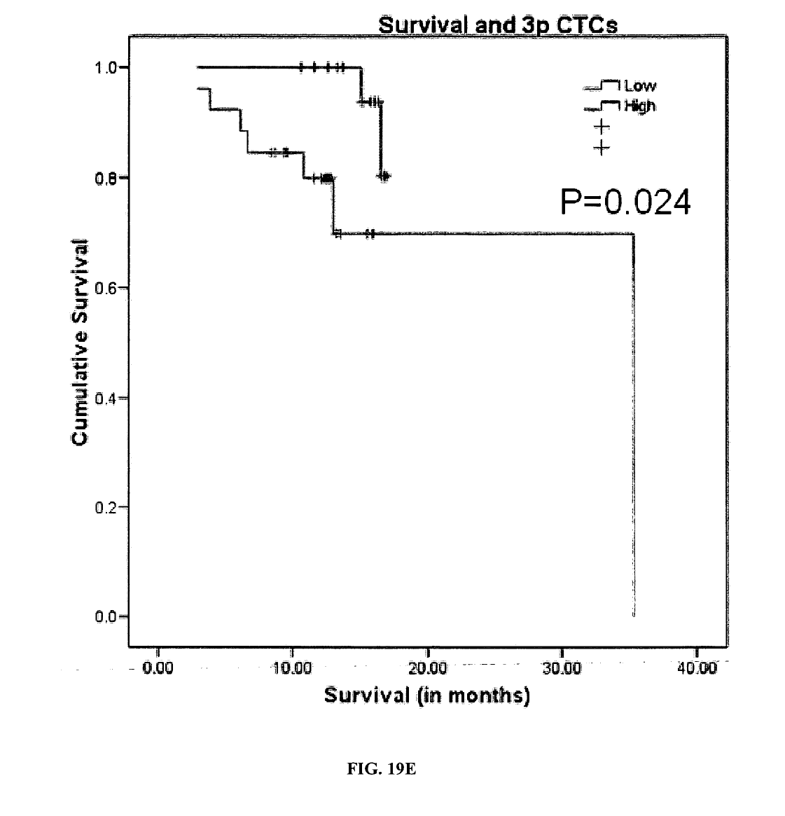

To evaluate the role of the biomarkers in survival, each marker was dichotomized at the median value (for all cases). Each dichotomy was evaluated using the Kaplan-Meier method (Table 26)(FIGS. 18A-E).

TABLE 24
Markers (P < 0.05) that are associated with overall survival

| Marker | | P* |
|---|---|---|
| 3p | Deletions | 0.006 |
| | Combined | 0.009 |

| | |
|---|---|
| 3p/10q Deletions | 0.032 |
| UroVysion 3p Deletions | 0.029 |
| Circulating Tumor Cells for 3p | 0.024 |

*P-value from log-rank test

TABLE 25
T test comparing variables between patients alive (42) or dead (13)

| | Alive/Dead Code | | | Std. | Std. Error |
|---|---|---|---|---|---|
| | Jan. 30, 2008 | N | Mean | Deviation | Mean |
| del 3p | Alive | 42 | 5.013 | 3.779 | 0.583 |
| | Dead | 13 | 9.118 | 4.864 | 1.349 |
| abn 3p/cep3 | Alive | 42 | 0.967 | 1.063 | 0.164 |
| | Dead | 13 | 0.588 | 0.653 | 0.181 |
| mono cep 3/3p | Alive | 42 | 0.721 | 0.748 | 0.115 |
| | Dead | 13 | 0.895 | 0.570 | 0.158 |
| poly cep 3/3p | Alive | 42 | 1.251 | 1.305 | 0.201 |
| | Dead | 13 | 1.732 | 1.382 | 0.383 |
| del 10q | Alive | 43 | 3.783 | 3.743 | 0.571 |
| | Dead | 12 | 6.057 | 5.359 | 1.547 |
| abn 10q/cep 10 | Alive | 43 | 1.446 | 1.550 | 0.236 |
| | Dead | 12 | 0.853 | 1.194 | 0.345 |
| mono cep 10/10q | Alive | 43 | 1.420 | 1.322 | 0.202 |
| | Dead | 12 | 1.028 | 1.184 | 0.342 |
| poly cep 10/10q | Alive | 43 | 0.540 | 0.535 | 0.082 |
| | Dead | 12 | 0.783 | 1.086 | 0.314 |
| del 3p + del 10q | Alive | 40 | 9.227 | 6.040 | 0.955 |
| | Dead | 12 | 15.102 | 8.297 | 2.395 |
| del + abn + mono + poly 3p | Alive | 42 | 7.951 | 4.166 | 0.643 |
| | Dead | 13 | 12.335 | 6.057 | 1.680 |
| del + abn + mono + poly 10q | Alive | 43 | 7.189 | 4.700 | 0.717 |
| | Dead | 12 | 8.720 | 5.742 | 1.657 |
| single del Lav | Alive | 45 | 7.544 | 4.237 | 0.632 |
| | Dead | 12 | 8.740 | 8.131 | 2.347 |
| EGFR Lav del | Alive | 45 | 1.296 | 1.333 | 0.199 |
| | Dead | 12 | 2.505 | 1.442 | 0.416 |
| 5p15.2 Lav del | Alive | 45 | 1.844 | 2.202 | 0.328 |
| | Dead | 12 | 2.641 | 3.200 | 0.924 |
| C-myc Lav del | Alive | 45 | 0.661 | 0.822 | 0.123 |
| | Dead | 12 | 0.803 | 1.056 | 0.305 |
| 6p11-q11 Lav del | Alive | 45 | 3.721 | 2.822 | 0.421 |
| | Dead | 12 | 2.831 | 4.371 | 1.262 |
| single gain Lav | Alive | 44 | 5.035 | 2.619 | 0.395 |
| | Dead | 12 | 7.676 | 5.618 | 1.622 |
| EGFR Lav gain | Alive | 45 | 3.362 | 2.289 | 0.341 |
| | Dead | 12 | 5.046 | 3.808 | 1.099 |
| 5p15.2 Lav gain | Alive | 45 | 0.552 | 0.822 | 0.123 |
| | Dead | 12 | 0.352 | 0.533 | 0.154 |
| C-myc Lav gain | Alive | 45 | 0.169 | 0.431 | 0.064 |
| | Dead | 12 | 0.556 | 0.789 | 0.228 |
| 6p11-q11 Lav gain | Alive | 45 | 0.943 | 1.162 | 0.173 |
| | Dead | 12 | 1.459 | 0.968 | 0.279 |
| % abn Lav | Alive | 45 | 2.216 | 1.871 | 0.279 |
| | Dead | 12 | 2.672 | 3.264 | 0.942 |
| single del Uro | Alive | 46 | 8.802 | 3.345 | 0.493 |
| | Dead | 13 | 9.048 | 3.466 | 0.961 |
| cep3 Uro del | Alive | 46 | 0.262 | 0.555 | 0.082 |
| | Dead | 13 | 0.331 | 0.407 | 0.113 |
| cep7 Uro del | Alive | 46 | 1.246 | 1.849 | 0.273 |
| | Dead | 13 | 1.353 | 1.859 | 0.516 |
| 9p21.3 Uro del | Alive | 46 | 1.527 | 1.924 | 0.284 |
| | Dead | 13 | 2.172 | 1.155 | 0.320 |
| cep17 Uro del | Alive | 46 | 5.751 | 2.775 | 0.409 |
| | Dead | 13 | 5.562 | 1.219 | 0.338 |
| single gain Uro | Alive | 46 | 4.998 | 3.538 | 0.522 |
| | Dead | 13 | 6.465 | 3.620 | 1.004 |
| cep3 Uro gain | Alive | 46 | 3.516 | 3.037 | 0.448 |
| | Dead | 13 | 3.382 | 2.822 | 0.783 |
| cep7 Uro gain | Alive | 45 | 0.377 | 0.531 | 0.079 |
| | Dead | 13 | 1.102 | 0.956 | 0.265 |
| 9p21.3 Uro gain | Alive | 46 | 0.580 | 0.670 | 0.099 |
| | Dead | 13 | 0.749 | 0.784 | 0.217 |
| cep17 Uro gain | Alive | 46 | 0.487 | 0.731 | 0.108 |
| | Dead | 13 | 1.418 | 1.569 | 0.435 |
| % abn Uro | Alive | 46 | 2.197 | 1.687 | 0.249 |
| | Dead | 13 | 1.926 | 1.204 | 0.334 |
| CTC/ul according to UroVysion | Alive | 44 | 21.137 | 25.595 | 3.859 |
| | Dead | 13 | 11.782 | 7.706 | 2.137 |
| CTC/ul according to LaVysion | Alive | 43 | 24.818 | 52.776 | 8.048 |
| | Dead | 12 | 12.729 | 13.305 | 3.841 |
| Lav + UroV Abnormal Cells | Alive | 45 | 4.404 | 2.856 | 0.426 |
| | Dead | 12 | 4.592 | 4.116 | 1.188 |
| CTC/ul according to 3p del | Alive | 40 | 52.255 | 80.043 | 12.656 |
| | Dead | 13 | 59.450 | 51.587 | 14.308 |
| CTC/ul according to abn3p/cep3 | Alive | 40 | 9.307 | 10.678 | 1.688 |
| | Dead | 13 | 3.895 | 6.993 | 1.939 |
| CTC/ul according to 10q | Alive | 41 | 33.722 | 48.283 | 7.541 |
| | Dead | 12 | 35.817 | 34.920 | 10.081 |
| CTC/ul according to abn10q/cep10 | Alive | 41 | 13.064 | 18.141 | 2.833 |
| | Dead | 12 | 6.661 | 14.712 | 4.247 |

TABLE 26
Means of CTCs between patients alive (46) or dead (13)

| | t-test for Equality of Means | | |
|---|---|---|---|
| | | 95% Confidence | |
| | | Interval of the | |
| | | Difference | |
| | Sig (2-tailed) | Lower | Upper |
| CTC/ul according to | 0.043 | 0.171 | 10.653 |
| abn3p/cep3 | | | |
| CTC/ul according to UroVysion | 0.038 | 0.514 | 18.195 |
| cep7 U₁₀ gain | 0.020 | −1.317 | −0.133 |
| del + abn + mono + poly 3p | 0.027 | −8.203 | −0.564 |
| del 3p + del 10q | 0.038 | −11.381 | −0.368 |
| EGFR Lav del | 0.008 | −2.091 | −0.326 |
| del 3p | 0.002 | −6.684 | −1.527 |

Similarly there were significant differences in biomarkers expressed in cells between alive (46) versus dead (13) patients (Table 27). Note that the most significant factors were deletions of deletion of 3p and deletions of the EGFR receptor. More patients who died have a loss of EGFR gene compared to patients alive, and this may have profound implications for therapy with anti-EGFR drugs.

TABLE 27
The effect of biomarkers on survival; Cox proportional hazard model

| | Beta | [symbol] tandard em [symbol] | t-value | beta | Wald stat. | p |
|---|---|---|---|---|---|---|
| cep17 Uro gain | 0.610935 | 0.181984 | 3.357089 | 1.842153 | 11.27005 | 0.000789 |
| del 3p | 0.148694 | 0.057365 | 2.592063 | 1.160318 | 6.718791 | 0.009545 |
| del 3p + del 10q | 0.104363 | 0.041077 | 2.540684 | 1.110003 | 6.455073 | 0.011068 |
| single gain Uro | 0.186415 | 0.077822 | 2.395399 | 1.204922 | 5.737938 | 0.016608 |
| del 10q | 0.146545 | 0.066709 | 2.196772 | 1.157826 | 4.825807 | 0.028044 |
| C-myc Lav gain | 0.721836 | 0.335441 | 2.151904 | 2.058209 | 4.630689 | 0.031412 |
| EGFR Lav del | 0.302965 | 0.142951 | 2.119364 | 1.353868 | 4.491702 | 0.034067 |
| 6p11-q11 Lav gain | 0.446170 | 0.220101 | 2.027111 | 1.562316 | 4.109178 | 0.042659 |
| del + abn + mono + poly3p | 0.101341 | 0.052340 | 1.936223 | 1.106654 | 3.748959 | 0.052849 |
| % abn Lav | 0.182009 | 0.105466 | 1.725752 | 1.199625 | 2.978219 | 0.084402 |
| 9p21.3 Uro del | 0.182181 | 0.110598 | 1.647238 | 1.199831 | 2.713395 | 0.099519 |
| del + abn + mono + poly10q | 0.092185 | 0.059666 | 1.545018 | 1.096568 | 2.387080 | 0.122352 |
| C-myc Lav del | 0.472250 | 0.314047 | 1.503753 | 1.603598 | 2.261273 | 0.132655 |
| CTC/ul according to abn3p/cep3 | −0.078914 | 0.052575 | −1.50097 | 0.924120 | 2.252917 | 0.133373 |
| polycep 10/10q | 0.525028 | 0.368507 | 1.424745 | 1.690506 | 2.029899 | 0.154241 |
| Lav + UroV Abnormal Cells | 0.123933 | 0.088170 | 1.405603 | 1.131940 | 1.975720 | 0.159852 |
| abn 3p/cep3 | −0.635531 | 0.458526 | −1.38603 | 0.529654 | 1.921078 | 0.165748 |
| 5p15.2 Lav del | 0.132808 | 0.095932 | 1.384405 | 1.142031 | 1.916576 | 0.166244 |
| single del Lav | 0.067338 | 0.049552 | 1.358932 | 1.069657 | 1.846696 | 0.174178 |
| EGFR Lav gain | 0.129912 | 0.103710 | 1.252643 | 1.138728 | 1.569113 | 0.210345 |
| 9p21.3 Uro gain | 0.322248 | 0.358288 | 0.899411 | 1.380228 | 0.808940 | 0.368441 |
| mono cep 10/10q | −0.298093 | 0.333089 | −0.894937 | 0.742232 | 0.800912 | 0.370828 |
| CTC/ul according to UroVysion | −0.018058 | 0.020678 | −0.873294 | 0.982104 | 0.762642 | 0.382510 |
| 5p15.2 Lav gain | −0.502045 | 0.598868 | −0.838323 | 0.605292 | 0.702786 | 0.401856 |
| cep3 Uro gain | −0.089656 | 0.107537 | 0.833722 | 1.093798 | 0.695092 | 0.404444 |
| CTC/ul according to abn10q/cep10 | −0.021617 | 0.027600 | −0.783244 | 0.978615 | 0.613470 | 0.433490 |
| abn 10q/cep 10 | −0.180393 | 0.258601 | −0.697575 | 0.834942 | 0.486611 | 0.485448 |
| CTC/ul according to 10q | 0.003940 | 0.006236 | 0.631827 | 1.003948 | 0.399205 | 0.527505 |
| single del Uro | 0.050963 | 0.084529 | 0.602904 | 1.052284 | 0.363493 | 0.546577 |
| cep3 Uro del | 0.222782 | 0.475081 | 0.468935 | 1.249548 | 0.219900 | 0.639120 |
| CTC/ul according to LaVysion | −0.005929 | 0.014380 | −0.412293 | 0.994089 | 0.169985 | 0.680128 |
| CTC/ul according to 3p | 0.001343 | 0.003486 | 0.385285 | 1.001344 | 0.148445 | 0.700029 |
| 6p11-q11 Lav del | −0.040162 | 0.115439 | −0.347903 | 0.960634 | 0.121037 | 0.727915 |
| mono cep 3/3p | 0.140486 | 0.414194 | 0.339179 | 1.150833 | 0.115043 | 0.734477 |
| cep7 Uro gain | −0.010907 | 0.036859 | −0.295920 | 0.989152 | 0.087568 | 0.767293 |
| % abn Uro | 0.049857 | 0.181200 | 0.275150 | 1.051121 | 0.075708 | 0.783203 |
| poly cep 3/3p | −0.040247 | 0.226533 | −0.177665 | 0.960552 | 0.031565 | 0.858988 |
| cep7 Uro del | 0.015506 | 0.141561 | 0.109534 | 1.015627 | 0.011998 | 0.912780 |
| single gain Lav | 0.001312 | 0.020993 | 0.062505 | 1.001313 | 0.003907 | 0.950161 |
| cep17 Uro del | −0.004555 | 0.123060 | −0.037015 | 0.995455 | 0.001370 | 0.970473 |

[symbol]

indicates data missing or illegible when filed

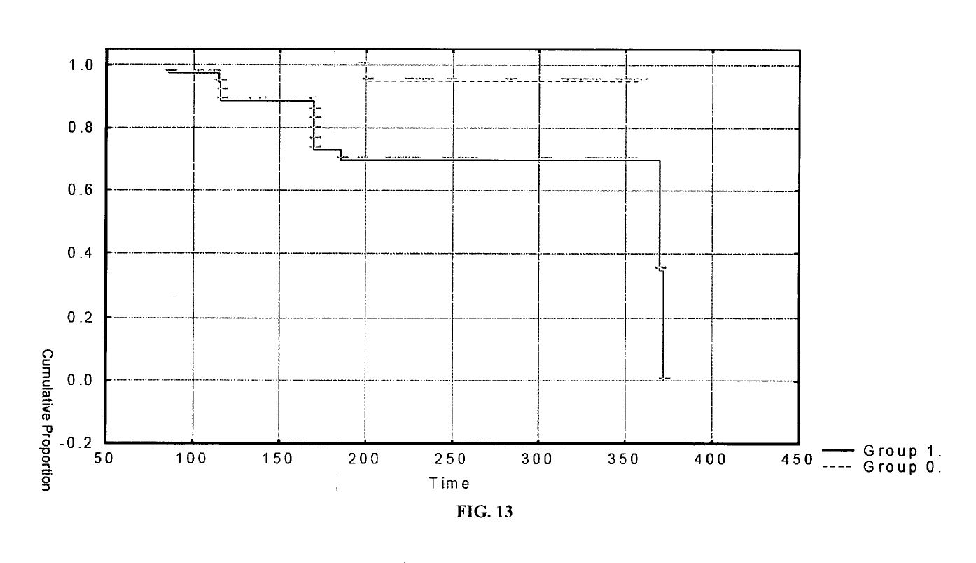

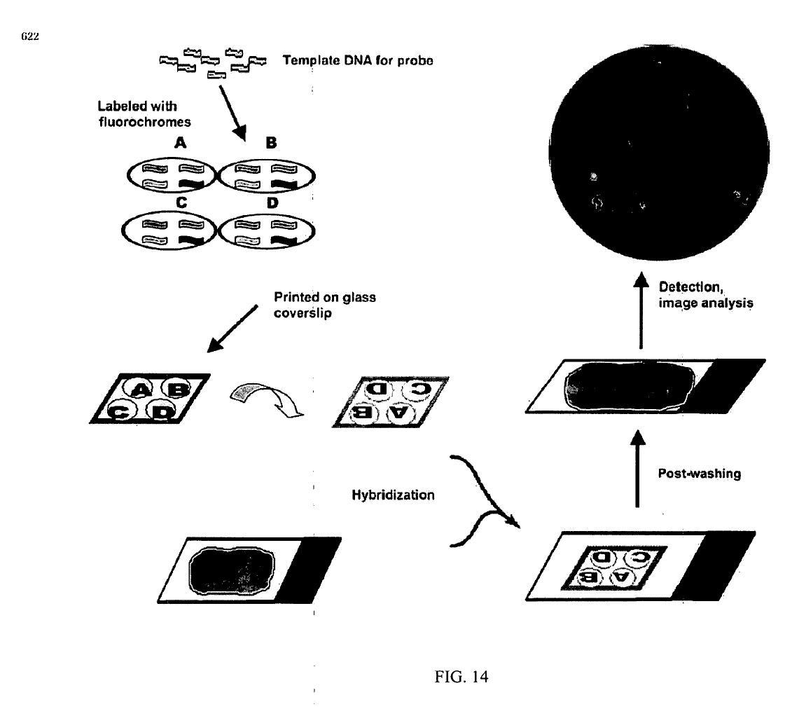

The above model shows the most important biomarkers ranked in order of significance according to survival. Note that in this model most important biomarkers are: 1) cep 17 gain; 2) deletion 3p; and 3) deletion 3p and 10q. In particular, the worse survival of patients was found with cep17_Uro gain (FIG. 13).

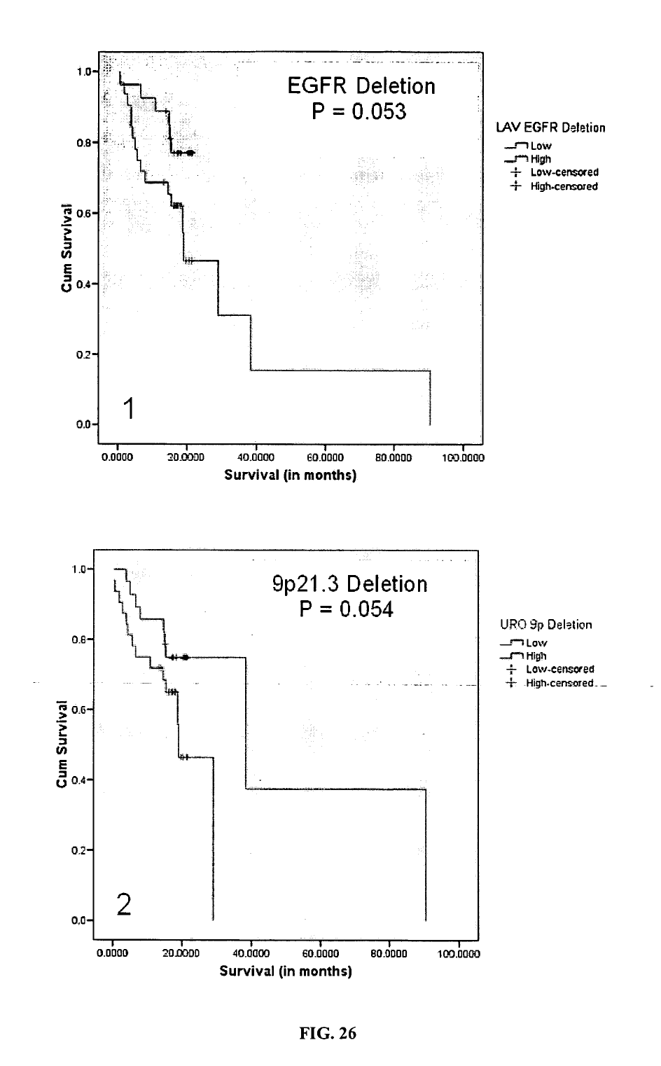

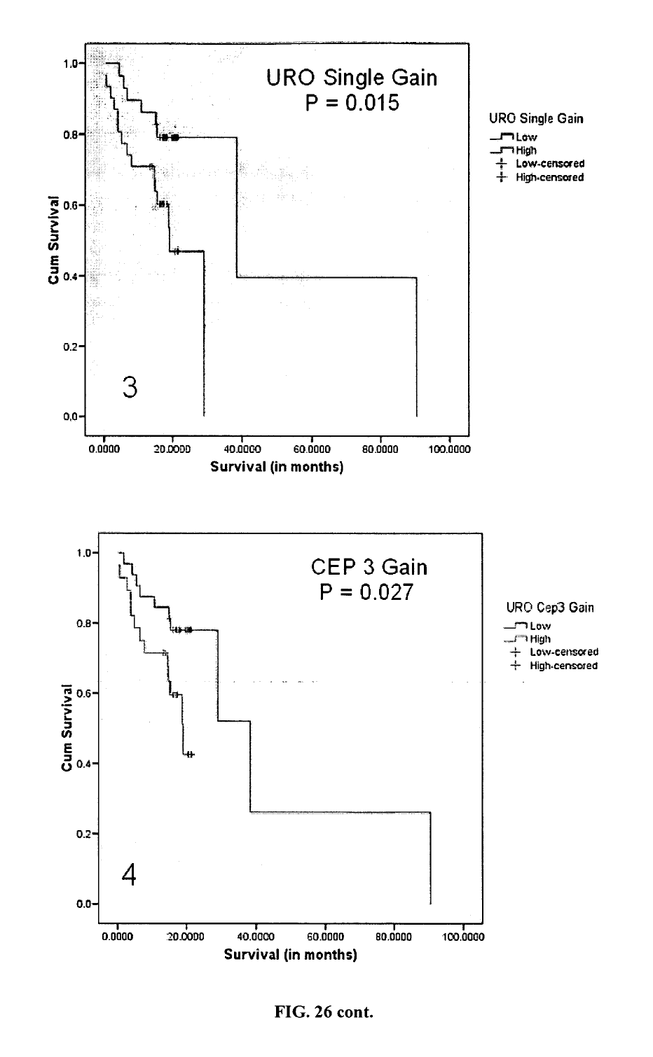

Twenty-two (37%) of the patients died over a period of less than 1 month to 39 months following collection of baseline blood samples. The median overall survival duration was 29 months (95% CI, 12.69 to 45.31 months). Six biomarkers were significant at the 10% level in Kaplan-Meier analyses for overall survival (FIG. 26B) but only two were significant at the univariate level: EGFR deletions and a single URO gain. However, these biomarkers were not significant for overall survival after adjustment for age, sex, and disease stage (Table 28).

TABLE 28
Markers (p < 0.10) associated with overall survival

| | | | | | Cox Model |
|---|---|---|---|---|---|
| | Median Survival (in months) | | | Cox Model | Adjusted |
| | 95% CI | | | Unadj HR | HR^{b} |
| Marker | Low | High | P^{a} | (95% CI) | (95% CI) |
| 10q22.3 Combined | 29.0 (9.9-48.1) | 19.2 (10.3-28.0) | 0.071 | — | — |
| Abnormalities | | | | | |
| 3p22.1/10q22.3 | 29.0 (—) | 18.9 (14.2-23.5) | 0.061 | 2.55 (1.02-6.38) | 1.32 (0.47-3.72) |
| Deletions | | | | | |
| Lav EGFR Deletions | — | 19.2 (9.5-28.9) | 0.053 | — | — |
| Uro 9p.21 Deletions | 28.4 (6.0-70.7) | 19.2 (14.9-23.4) | 0.054 | — | — |
| Single Gain | 38.4 (6.2-70.6) | 18.9 (14.4-23.4) | 0.015 | 3.62 (1.38-9.50) | 2.51 (0.89-7.06) |
| Cep 3 Gain | 29.0 (10.0-48.0) | 18.9 (15.2-22.6) | 0.027 | — | — |

^{a}P-value from Kaplan-Meier log-rank test,.
^{b}HR adjusted by age, sex and stage;
— Median survival estimates not calculable.

### Example 8

### Enrichment of the Sample

The sample may be enriched prior to the FISH analysis by staining for CD45. The CD45-positive, CD45-negative and combined readings are all significant for different markers (see Tables 29, 30, and 31).

TABLE 29
CD45-Negative Cells

Number of Cases

| No Cancer vs Cancer | p-value | Control | Cancer |
|---|---|---|---|
| Lav % abn | 0.0005 | 7 | 10 |
| CTC/ul according to | 0.008 | 7 | 10 |
| LaVysion | | | |
| 3p del | 0.008 | 11 | 11 |
| 3p/cep3poly | 0.010 | 11 | 11 |
| Lav cep6 del | 0.019 | 7 | 8 |
| 10q del | 0.029 | 12 | 8 |
| Uro 9p21 del | 0.042 | 4 | 7 |
| CTC/ul according to | 0.052 | 5 | 9 |
| UroVysion | | | |

Number of Cases

| Stage IB Stage IV | p-value | IB | IV |
|---|---|---|---|
| Uro single del | 0.032 | 1 | 6 |

Number of Cases

| Stage IIIA-IIIB vs Stage IV | p-value | IIIA-IIIB | IV |
|---|---|---|---|
| Uro 9p21 del | 0.019 | 1 | 6 |

TABLE 30
CD45-Positive Cells

Number of Cases

| No Cancer vs Cancer | p-value | Control | Cancer |
|---|---|---|---|
| Lav single del | 0.0001 | 7 | 10 |
| 3p/cep3poly | 0.001 | 11 | 11 |
| 3p del | 0.001 | 11 | 11 |
| Lav single gain | 0.002 | 7 | 10 |
| 10q del | 0.005 | 12 | 8 |
| Uro 9p21 del | 0.006 | 4 | 7 |
| Lav % abn | 0.006 | 7 | 10 |
| Lav cep6 gain | 0.019 | 7 | 8 |
| Lav EGFR del | 0.023 | 7 | 8 |
| 3p/cep3 abn | 0.026 | 11 | 11 |
| Lav EGFR gain | 0.033 | 7 | 8 |
| Lav c-myc del | 0.036 | 7 | 8 |
| CTC/ul according to | 0.046 | 7 | 10 |
| LaVysion | | | |
| CTC/ul according to 3p | 0.048 | 10 | 11 |

Number of Cases

| Stage IA vs Stage IIIA-IIIB | p-value | IA | IIIA-IIIB |
|---|---|---|---|
| CTC/ul according to | 0.008 | 1 | 2 |
| LaVysion | | | |

TABLE 31
CD45-Positive and -Negative Combined

Number of Cases

| No Cancer vs Cancer | p-value | Control | Cancer |
|---|---|---|---|
| Lav % abn | 0.001 | 7 | 10 |
| Lav cep6 del | 0.001 | 7 | 8 |
| 3p del | 0.002 | 11 | 11 |
| 3p/cep3poly | 0.005 | 11 | 11 |
| CTC/ul according to | 0.008 | 7 | 10 |
| LaVysion | | | |
| Uro 9p21 del | 0.015 | 4 | 7 |
| 3p/cep3 mono | 0.018 | 11 | 11 |
| 10q del | 0.023 | 12 | 9 |
| Uro single gain | 0.050 | 5 | 9 |

Material and Methods for Blood Samples for Quantitation of CD45 Positive (lymphocytes) and Negative CTCs. Peripheral blood from patients with lung cancer (at least 2 ml of blood) is subjected gradient separation via Ficoll Hypaque centrifugation. Cytospins of the recovered peripheral blood mononuclear cells (PBMCs), encompassing lymphocytes, stem cells, monocytes and circulating tumor cells, are prepared using a Shandon cytospin. Multiple slides (that have been sialine coated to prevent loss of cells) are prepared from the enriched fraction and spray fixed with an alcohol preservative spray. Each slide contains from 10,000 to 15,000 PBMCs. One Diff quick slide is made to check the cellularity of the Cytospins. When the optimal concentration has been achieved, preparations are stained with a FITC conjugated -CD45 (Becton Dickinson) at 1:25 dilution using antigen retrieval with citric buffer and then subjected to steam for 30 minutes. Slides are incubated for 2 hours, washed and then DAPI is applied. Slides are then scanned on the Bioview instrument until at least 5000 cells have been quantitated. Following this, the operator will visually select as many CD45 negative cells as possible. Usually 150 to 200 CD45 negative cells are obtained. These selected cells are then placed into a “target” category according to a specialized software program. Similarly, at least 500 CD45 bright cells are selected and placed in a second “target” category for later recall. Following scanning and categorization of cells into CD45 positive and negative classes, a hard-copy of the CD45 positive and negative cells is then made.

The same slide that has been stained with the CD45 antibody is subsequently subjected to FISH using the described DNA probes using the standard FISH protocol for the two commercial probes UroVysion and LaVysion and the probes for CEP3/3p22.1 and CEP10, 10q22.3. Slides are hybridized overnight. Hybridized slides are scanned on Duet, Bioview automated scanner using a dedicated software program that matches each hybridized FISH positive cell with the same original saved CD45 negative cell. The procedure is repeated for analyzing the CD45 positive cells with matching of the CD45 positive stored cell image with the identical cell that has been subsequently hybridized with a selected FISH probe.

The numbers of cells analysed in this way ranged from 80 cells to 450 cells, however usually at least 150 CD45 negative cells (which are present in much lower percentages than CD45 positive cells) and 300 CD45 positive cells are analysed. CD45 positive and CD45 negative cells are then classified separately into normal, deletions, gains, monosomies and polysomies of chromosomes or genes, according to the number of signals for each biomarker. Results are then entered into a spread sheet and expressed as a percentage of cytogenetically abnormal cells (CACs) for each class of abnormality. To calculate circulating tumor cells (CTCs), the formula (percentage CACs X total number of PBMCs divided by volume of blood per ml, divided by 1000) is used to express the CTCs per microliter.

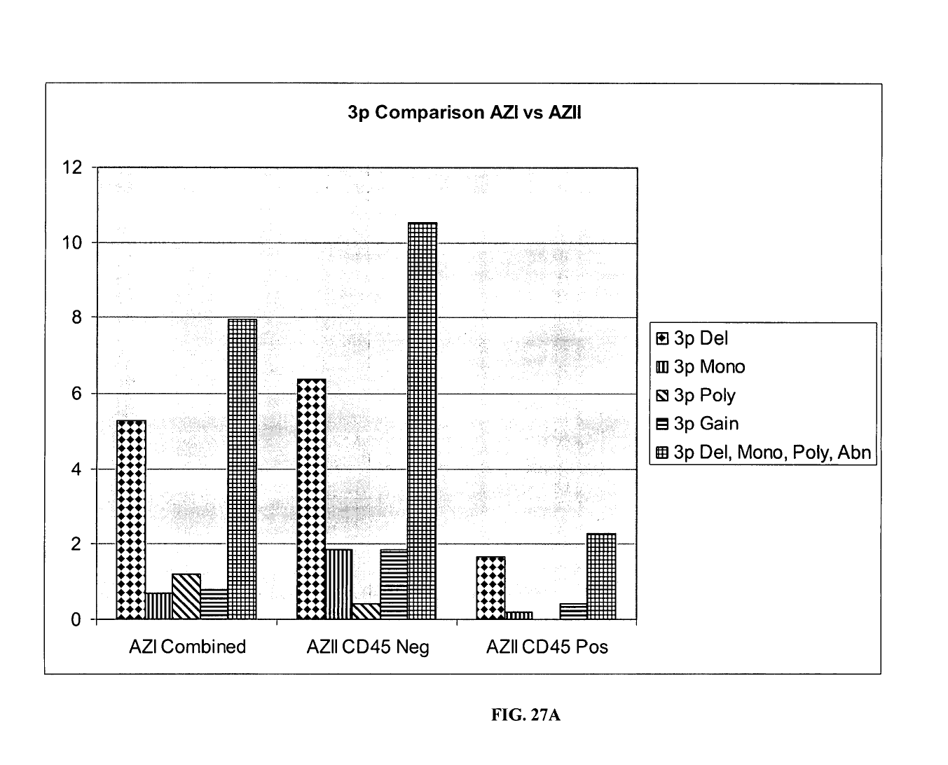

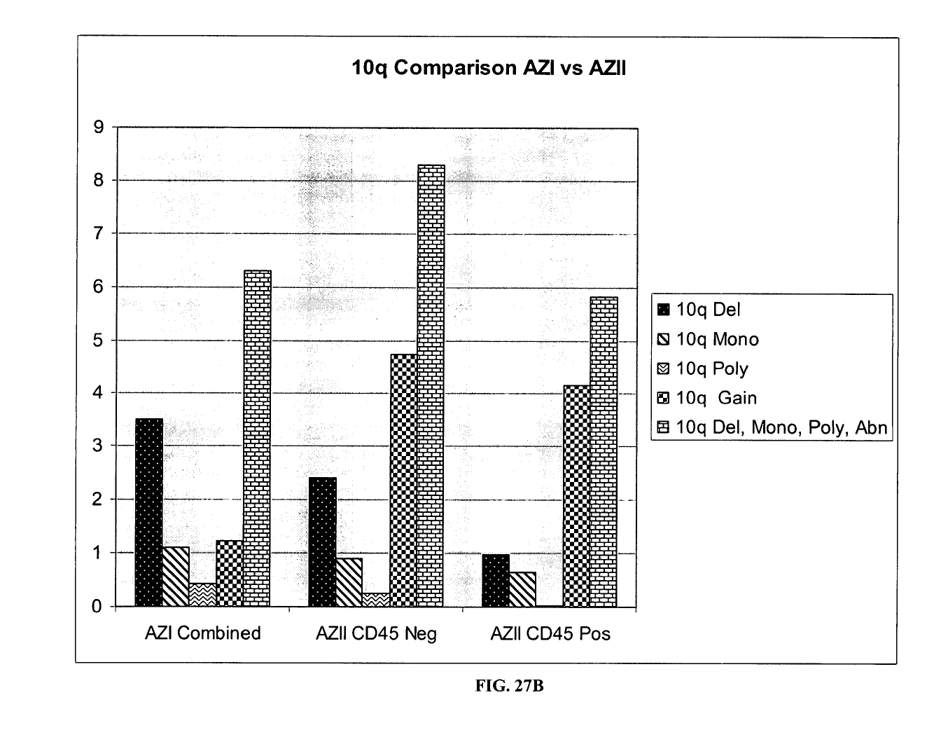

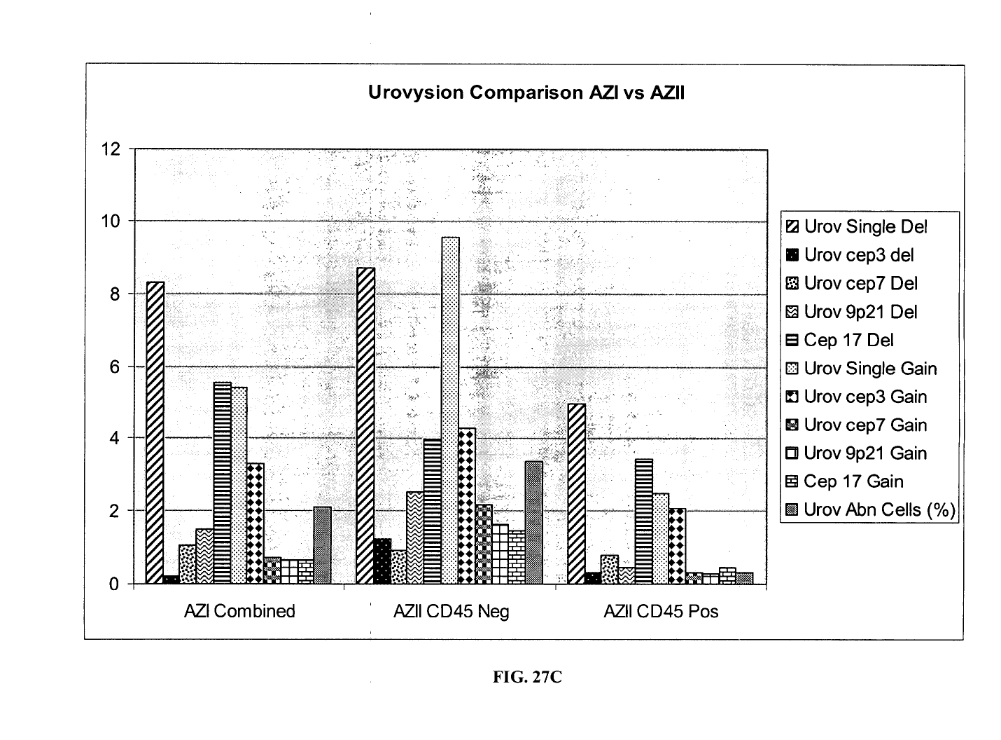

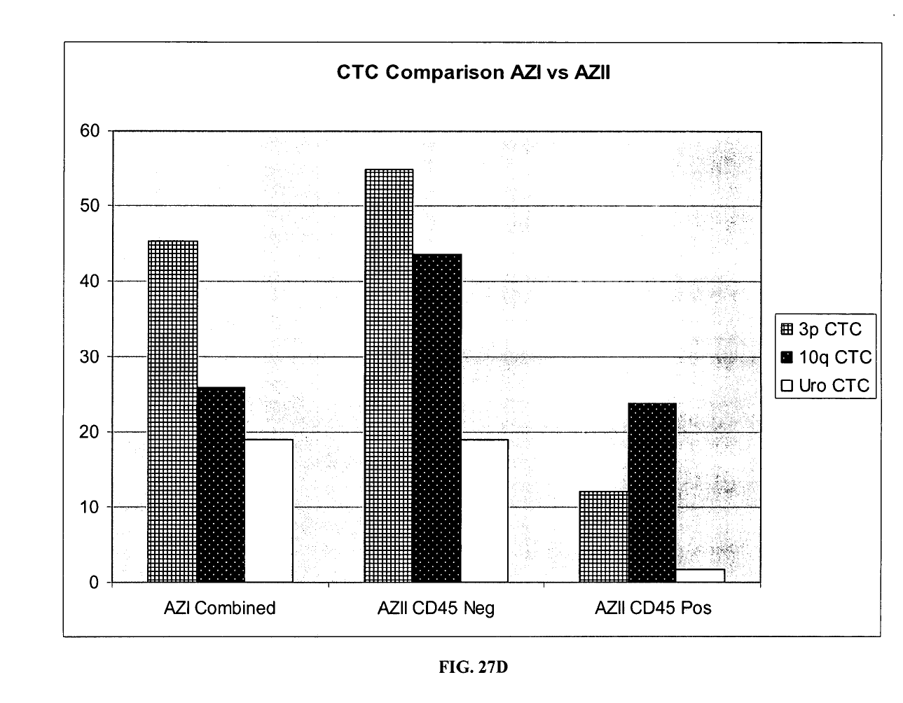

AZI refers to a sample that was not enriched prior to the FISH analysis by staining for CD45. AZII refers to a sample that was enriched prior to the FISH analysis by staining for CD45. See FIGS. 27A-D and Tables 32-35.

TABLE 32
3p Comparison AZI vs AZII

| | | | | 3p | 3p Del, Mono, |
|---|---|---|---|---|---|
| | 3p Del | 3p Mono | 3p Poly | Gain | Poly, Abn |
| AZI Combined | 5.29 | 0.68 | 1.18 | 0.79 | 7.94 |
| AZII CD45 Neg | 6.39 | 1.86 | 0.42 | 1.86 | 10.53 |
| AZII CD45 Pos | 1.66 | 0.19 | 0.01 | 0.42 | 2.29 |

TABLE 33
10q Comparison AZI vs AZII

| | | | | | 10q Del, |
|---|---|---|---|---|---|
| | | 10q | 10q | | Mono, Poly, |
| | 10q Del | Mono | Poly | 10q Gain | Abn |
| AZI Combined | 3.5 | 1.11 | 0.43 | 1.23 | 6.28 |
| AZII CD45 Neg | 2.41 | 0.9 | 0.26 | 4.74 | 8.3 |
| AZII CD45 Pos | 0.97 | 0.66 | 0.02 | 4.16 | 5.81 |

TABLE 34
Urovysion Comparison AZI vs AZII

| | | | | | | | | | | | Urov |
|---|---|---|---|---|---|---|---|---|---|---|---|
| | Urov | Urov | Urov | | | Urov | Urov | Urov | Urov | Cep | Abn |
| | Single | cep3 | cep7 | Urov | Cep | Single | cep3 | cep7 | 9p21 | 17 | Cells |
| | Del | del | Del | 9p21 Del | 17 Del | Gain | Gain | Gain | Gain | Gain | (%) |
| AZI Combined | 8.29 | 0.2 | 1.05 | 1.51 | 5.53 | 5.4 | 3.3 | 0.72 | 0.65 | 0.65 | 2.12 |
| AZII CD45 Neg | 8.69 | 1.21 | 0.92 | 2.5 | 3.97 | 9.55 | 4.28 | 2.18 | 1.62 | 1.47 | 3.36 |
| AZII CD45 Pos | 4.96 | 0.32 | 0.77 | 0.44 | 3.43 | 2.49 | 2.09 | 0.32 | 0.28 | 0.44 | 0.32 |

TABLE 35
CTC Comparison AZI vs AZII

| | 3p CTC | 10q CTC | Uro CTC |
|---|---|---|---|
| AZI Combined | 45.42 | 25.84 | 18.92 |
| AZII CD45 Neg | 54.9 | 43.72 | 18.97 |
| AZII CD45 Pos | 12.07 | 23.74 | 1.73 |

### Example 9

### FISH-Based Finger-Stick Blood Test

Because there was such a large significant difference in biomarkers between cases versus controls for all stages of cancer, and cancer versus controls for stage 1A cancer of lung, it is possible to reduce number of markers tested to just the most significant ones to determine cancer versus no cancer status.

A 4-color FISH array with 2 spots for interphase multi-color FISH is synthesized. SPOT A contains Cep10/10q22.3 SP-A gene, cep3/3p22.1 GC20 gene; and SPOT B contains CEP7/7p22.1 EGFR gene, cep17, 9p21.3. The probes is labeled as in Example 1 with red, green, gold and aqua fluorochromes. Using the technology described by Li et al. (2006), the cocktail of probes precipitated with COT DNA is suspended in a polyacrilamide gel or into a slide with several wells in hybridization buffer for subsequent hybridization to a monuclear suspension of cells previously labeled with CD45. Two different spots or wells contain the probes of interest. Either manual counting or an automated image analyzer is used to score the CD45 diminished or negative cells labeled with the different FISH cocktails. Results are input and an algorithm is applied to the previously set up ROC curve to obtain probability of cancer versus no cancer. According to the FISH results as described in this application, sensitivity and specificity is 97% using this probe combination.

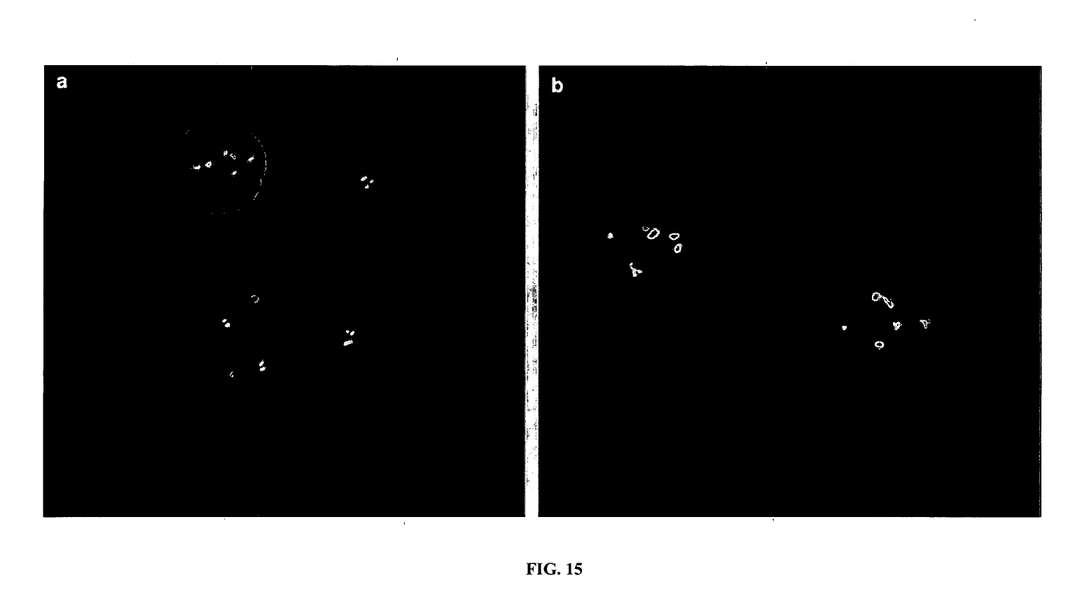

FIGS. 14 and 15 are examples of the slide micro-array technique taken from Li et al. (2006), herein incorporated by reference in its entirety.

### Example 10

### Methods

Genetic markers are applied (following Ficoll Hypaque gradient separation to isolate mononuclear cells) to the CD45-negative, diminished and positively staining peripheral blood mononuclear cells following an antibody reaction with FITC-conjugated CD45 antibody. CD45 will stain the peripheral blood mononuclear cells (lymphocytes, monocytes) positive, while circulating tumor cells are stained dimly or not at all. The molecular probes used are:

Three probes were developed for use with the present invention. See U.S. Pat. No. 6,797,471 and U.S. Publication No. 2007/0218480, incorporated herein by reference in their entirety. The three probes included a 10q22-23 probe, which encompasses surfactant protein A1 and A2 combined with centromeric 10; a 2p22.1 probe, which is a nucleic acid probe targeting RPL14, CD39L3, PMGM, or GC20, combined with centromeric 3; and PI3kinase.

Commercial probes for use with the current invention include the UroVysion DNA probe set (available from Vysis/Abbott Molecular, Des Plaines, Ill.) which includes probes to centromeric 3, centromeric 7, centromeric 17, and 9p21.3. Another set of commercial probes is the LaVysion DNA probe set (also available from Vysis/Abbott Molecular, Des Plaines, Ill.) which includes probes to 7p12 (epidermal growth factor receptor), 8q24.12-q24.13 (MYC), 6p11.1-q11(chromosome enumeration (Probe CEP 6), and 5p15.2 (encompassing the SEMA5A gene). A third commercially available probe set is a single probe set Centromeric7/7p12 (epidermal growth factor receptor). 10q22.3, and 3p22.1, as well as the UroVysion probes, are useful to detect early changes of lung cancer. In contrast, the LaVysion probe set detects higher stages or more advanced stags of lung cancer.

Using an automated fluorescence scanner (Bioview, Rehovoth, Israel) with dedicated software, specific for the FISH probes, several thousands of CD45-positive, diminished and negative cells were scanned from each patient, and then hybridized with the above probes and rescanned. The CD45-positive, CD45-negative and combined readings are all significant for different markers. The FISH and fluorescent images were then matched up and displayed side by side. An operator then examined each cell interactively to confirm loss or gain of fluorescent signals, and that each cell was isolated and not overlapped by a neighbouring cell. In the case of the 4-color FISH multiple filters are used to examine different color signals. Special care has to be taken to avoid misinterpreting “split” signals. Subsequently the CD45-stained image, irrespective of presence or absence of fluorescence, together with the multicolored signals from the FISH image were analysed, quantitated and tabulated in a pie chart. Total signals were expressed as a percentage of cells with deletions or polysomies of each tested gene. In addition cases were also analysed to enrich CTC counts by counting abnormalities only in CD45-diminished and -negative cells (the fraction containing the putative tumor cells) (CD45DN) and expressing these as percentages of the CD45DN population.

Results were also calculated via a special formula developed in the laboratory based on initial total mononuclear cell count, percentage of abnormal cells and correction for dilution factors, for each molecular probe to demonstrate the number of abnormal cells or CTCs per microliter of blood.

Results of patients' variables versus controls were analyzed via a variety of statistical combinations, including Chi-squared tests and Pearson's correlation, a non parametric Mann-Whitney test was used to identify probes that can be used to distinguish cases and controls. A binary logistic regression model and a backward Likelihood Ratio model were used to predict case control status and a Cox proportional hazard modelchose the best biomarkers that predicted for survival.

In addition, a ROC analysis was performed to discover which molecular biomarkers were most predictable of cases versus controls.

### Preparation of the 111299 Cell Line for Recovery Experiments

The H1299 cell line was cultured in accordance with American Type Culture Collection (Manassas, Va.) guidelines. Cells were counted using a Coulter counter. Seven thousand cells per milliliter (1% mixture) and 25,000 cells/ml (5% mixture) were spiked into blood specimens to estimate the percentage of H1299 cells recovered by different FISH probes.

### Study Population

In 2007 and 2008, peripheral blood specimens were collected prospectively from 59 patients with NSCLC and 24 controls including heavy smokers at high risk for lung cancer at The University of Texas M. D. Anderson Cancer Center under an Institutional Review Board-approved protocol.

### Demographic Characteristics of the Study Population

Criteria for study entry included no treatment prior to surgery for stage I-III NSCLC cases. Equal stratification of patients across all NSCLC stages was attempted. Corresponding primary lung tumor tissue specimens were available for 21 patients. Mean ages of the controls and patients were 55.5±2.86 and 66.8±1.36 years, respectively (Table 36). Disease stages ranged from low (14 IA, 8 IB, and 9 II) to high (10 III and 18 IV), and adenocarcinoma was the most common subtype. Of 23 patients who had a relapse or persistent disease, 4 had an early relapse (within 6 months to 1 year after first treatment). At the time of data analysis, 22 patients had died, most of whom had stage III or IV disease.

TABLE 36
Demographic characteristics of the study population

| | | | Clinical Stage | | | | |
|---|---|---|---|---|---|---|---|
| | Controls | Cases | IA | IB | II | III | IV |
| Characteristic | N = 24 | N = 59 | N = 16 | N = 8 | N = 7 | N = 10 | N = 18 |
| Gender: N (%) Male | 9 (37.5) | 30 (50.8) | 11 (78.6) | 4 (50.0) | 2 (22.2) | 5 (50.0) | 8 (44.4) |
| P* | | 0.269 | | | | | |
| Age (Mean ± SE) | 55.5 ± 2.86 | 66.8 ± 1.36 | 67.4 ± 2.55 | 70.63 ± 3.67 | 61.0 ± 3.01 | 67.7 ± 2.94 | 66.9 ± 2.88 |
| P* | | 0.001 | | | | | |
| Histology: N (%) | | | | | | | |
| Squamous | | 12 (20.3) | 6 (37.5) | 2 (25.0) | 2 (28.6) | 1 (10.0) | 1 (5.6) |
| Adenocarcinoma | | 32 (54.2) | 9 (64.3) | 5 (62.5) | 5 (71.4) | 4 (40.0) | 9 (50.0) |
| NSC | | 15 (25.4) | 1 (7.1) | 1 (12.5) | 0 (0.0) | 5 (50.0) | 8 (44.4) |
| Relapse: N (%) with positive | | 23 (39.0) | 1 (7.1) | 1 (12.5) | 0 (0.0) | 3 (30.0) | 18 (100.0) |
| relapse | | | | | | | |
| Early Relapse: N (%) with positive | | 4 (6.8) | | | | | |
| relapse (between 6 mo and 1 year) | | | | | | | |
| Vital Status: N (%) Deceased | | 22 (37.3) | 1 (4.5) | 0 (0.0) | 0 (0.0) | 6 (27.3) | 15 (68.2) |

*P-value derived from Mann-Whitney test (continuous variables); Chi-square test for association (categorical variables); all P-values are two-sided and compare controls to all cases; NSC = non-small cell carcinoma

### Specimen Collection

Twenty-five mL of blood was collected from each subject in vacutainers with ethylenediaminetetraacetic acid (EDTA) as an anticoagulant and subjected to Ficoll-Hypaque density gradient separation. Peripheral blood mononuclear cells (PBMCs) were isolated and counted using a Coulter counter (Beckman Coulter, Fullerton, Calif.), and cytospin preparations of PBMCs containing an average of 10,000 cells were prepared. PBMCs from all 59 patients and 24 controls were tested with the same panel of biomarkers. From the above 59 patients, tumor tissue was available on 21 patients who were enrolled in a lung cancer Specialized Program of Research Excellence study. FISH was performed on both the peripheral blood and tumor tissue to detect concordance of genetic abnormalities in both surrogate and target tissues.

### Preparation of Blood Specimens for Isolation of Cytogenetically Abnormal Cells (CACS)

Blood specimens obtained from healthy donors were processed using the Lymphoprep separation medium in order to isolate mononuclear cells by density gradient centrifugation method (Ficoll; Axis-Shield, Oslo, Norway). Cells were passed through a 50-μm mesh tube, and cell numbers in the sample were quantitated using a Coulter counter.

### Preparation of Slides for Spiked Cells (Adenocarcinoma Cell Line Plus Donor Cells)

Two different concentrations of spiked cells were prepared with 40,000 cells each. Slides with 1% concentration had 100 μL solution containing 39.6 μL of donor cell solution, 57.14 μL of H1299 cells and 3.2 μL of 1×PBS. Slides with 5% concentration of spiked cells had 100 μl solution containing 19 μL of donor cells, 80 μL of H1299 cell line and 1 μL of 1×PBS. Cytospins of spiked cells were prepared using a Shandon Cytospin 3 (Shandon Inc, Pittsburgh, Pa.) at 750 RPM for 3 minutes. Slides were spray fixed air dried and stored at −20° C.

### Definitions of Biomarker Abnormalities

Using the dual probe sets, a deletion was defined as loss of the locus-specific probes 3p22.1 or 10q22.3 compared with the internal centromeric control probes (CEP3) or (CEP10), a gain was defined as an extra copy of 3p22.1 or 10q22.3 relative to the corresponding centromeric probes, for example 3 copies of 3p22.1 relative to 2 copies of CEP3, or 3 copies of 10q22.3 relative to 2 copies of CEP10. Monosomy was defined as a single copy of CEP3 or CEP10 with loss of the corresponding locus-specific probe, polysomy was defined as extra copies of CEP3/3p22.1 or 10q22.3/CEP10. Combined abnormalities were the sum of deletions, gains, monosomies and polysomies. Using the four-color probe sets, normal cells were defined as diploid for all four probes if two red, two green, two aqua, and two yellow signals were present in the nuclei of the PBMCs. Abnormal cells were defined as those with at least two chromosomal abnormalities (either gain or loss). A single chromosomal gain was defined as the presence of an extra signal for a total of nine signals, and a single chromosomal loss was defined as the loss of a signal for a total of seven signals.

### FISH Testing for Cytogenetically Abnormal PBMCs (CACs)

The following panel of FISH probes was used: 1) a combination of two probe sets: Locus Specific Identifier (LSI) 3p22.1 with corresponding centromeric probe CEP3 and LSI 10q22.3 [SP-A] with corresponding CEP10 prepared in-house as described previously (Katz et al., 2008; Barkan et al., 2005; Yendamuri et al., 2008) and 2) two commercially available probe sets containing four probes each—LAVysion [LAV]: EGFR, C-MYC, 6p11-q11, and 5p15.2; and UroVysion [URO]: CEP3, CEP7, CEP17, and 9p21.3 (Abbott Molecular, IL). Fluorescent signals in specimens were quantitated on a per-cell basis using an automated fluorescent system (Bioview, Rehovoth, Israel) that is capable of scanning and classifying hundreds of cells under fluorescent illumination and allows for detection of rare cells according to FISH pattern (Daniely et al., 2005). Using two-color FISH with 3p22.1/CEP3 and 10q22.3/CEP10 a mean of 250 PBMCs was accumulated for each probe set and reviewed for appropriate morphology (round or oval cells) and to verify the number of FISH signals displayed by the program on a per-cell basis by an experienced observer blinded to the disease status. Similarly, at least 200 PBMCs were selected and scored for genomic abnormalities using both URO or LAV four-color probe sets. Cytogenetic abnormalities were scored based on the presence of chromosomal deletions, gains, monosomy, polysomy, or the sum of all abnormalities combined and expressed as percentages of CACs.

### Fluorescence In-Situ Hybridization (FISH)

FISH was performed, using the standard FISH protocol for all four probe sets (3p22.1/CEP3, 10q22.3/CEP10, URO and LAV) for CTCs and tumor wash cells. The average number of cells classified for CTCs for 3p22.1/CEP3 were 218; for 10q22.3/CEP10 283; LaVysion, 225; and UroVysion, 254. The average number of cells classified for tumor wash for 3p22.1/CEP3 were 213; for 10q22.3/CEP10 213; LaVysion, 159; and UroVysion, 145 (range 50 to 450 cells) (Table 37). Cells were classified exactly according to the scheme used for scoring the CTCs.

TABLE 37
Number of Cells Classified (Mean ± Standard Deviation) for CTCs

| | 3p22.1/CEP3 | 10q22.3/CEP10 | LAVysion | UroVysion |
|---|---|---|---|---|
| Controls | 181 (±61) | 275 (±217) | 188 (±83) | 207 (±106) |
| CTCs | 218 (±70.1) | 283 (±123) | 225 (±93.17) | 254 (±194) |
| Tumor Wash | 213 (±98.5) | 213 (±81.9) | 159 (±22.9) | 145 (±28.4) |

Before specimen slides were pretreated, they were fixed in fresh Carnoy's fixative (3 parts Methanol: 1 part Acetic acid) for 30 minutes at room temperature. Slides were pretreated at 73° C. with 2×SSC for 2 minutes, and then digested with 0.5 mg/ml protease solution at pH of 2.00 for 5 minutes for blood cells or 10 minutes for tumor wash cells at 37° C. Slides were washed with 1×PBS, fixed in 1% Formaldehyde and were then rinsed in 1×PBS for 5 minutes each at room temperature. Serial ethanol dehydration was done (70%, 85%, and 100%) for 2 minutes each and the slides were air-dried at room temperature. Four Probe sets were used: [3p22.1 (Spectrum Green; prepared in-house)/CEP3 (Spectrum Orange; Abbott Molecular), 10q22.3 (Spectrum Green; prepared in-house)/CEP10 (Spectrum Orange; Abbott Molecular), UroVysion consisting of CEP3 (Spectrum Red), CEP7 (Spectrum Green), CEP17 (Spectrum Aqua) and LSI 9p21 (Spectrum Yellow) and LAVysion consisting of LSI 5p15.2 (Spectrum Green), CEP6 (Spectrum Aqua), LSI 7p12 (Spectrum Red) and LSI 8q24 (Spectrum Yellow); Abbott Molecular, IL)] Required probe was applied on each slide. The coverslip was placed, which was then sealed with rubber cement. The slides and the probe were co-denatured by placing the slides on the surface of a 73° C. prewarmed plate (HYBrite, Abbott Molecular) for 5 minutes and hybridized (16-20 hours) overnight at 37° C. Next day, the coverslips were carefully removed and were washed in 73° C. preheated post hybridization wash buffer 0.4×SSC/0.3% Nonidet P-40, for 2 minutes and rinsed at room temperature for 1 minute in 2×SSC/0.1% Nonidet P-40. Slides were then counterstained with 10 μl of 14 μg/ml 4,6-diaminidino-2-phenylidole (DAPI, Boehringer Manheim, Indianapolis, Ind.) in the mounting medium Vectashield (Vector Laboratories, Burlingame, Calif.), and a coverslip was applied.

TABLE 38
Recovery Experiments Spiking Lung Cancer Cell Line into Peripheral Blood Mononuclear Cells (PBMCs)
Using Different Biomarkers

| | | | | | | Expected Tumor | Actual Tumor | | Number of |
|---|---|---|---|---|---|---|---|---|---|
| | Normal | Deletion | Gain | Monosomy | Polysomy | Cell Recovery | Cell Recovery | | Cells |
| | (%) | (%) | (%) | (%) | (%) | (%) | (%) | Yield % | Analyzed |
| 3p22.1 Unspiked | 99.6 | 0.4 | 0 | 0 | 0 | 1 | 0.4 | 40 | 500 |
| PBMC | | | | | | | | | |
| 3p22.1 Cell Line | 0.4 | 0 | 0 | 0 | 99.6 | 100 | 99.6 | 99.6 | 500 |
| 3p22.1 1% Dilution | 99.2 | 0.2 | 0 | 0 | 0.6 | 1 | 0.4 | 40 | 500 |
| 3p22.1 5% Dilution | 96 | 0.2 | 0 | 0 | 3.8 | 5 | 3.6 | 72 | 500 |
| 10q22.3 Unspiked | 99.8 | 0.2 | 0 | 0 | 0 | 1 | 0.2 | 20 | 500 |
| PBMC | | | | | | | | | |
| 10q22.3 Cell Line | 0.27 | 0 | 0 | 0 | 99.7 | 100 | 99.7 | 99.7 | 372 |
| 10q22.3 1% Dilution | 99.2 | 0.2 | 0 | 0 | 0.6 | 1 | 0.6 | 60 | 500 |
| 10q22.3 5% Dilution | 96.4 | 0 | 0.4 | 0.2 | 3 | 5 | 3.4 | 68 | 500 |

| | | | | | Expected Tumor | Actual Tumor | | Number of |
|---|---|---|---|---|---|---|---|---|
| | Normal | Loss | Gain | Abnormal | Cell Recovery | Cell Recovery | | Cells |
| | (%) | (%) | (%) | (%) | (%) | (%) | Yield % | Analyzed |
| LAV Unspiked | 99 | 0.5 | 0.5 | 0 | 1 | 0 | 0 | 200 |
| PBMC | | | | | | | | |
| LAV Cell Line | 0.5 | 0 | 0 | 99.5 | 100 | 99.5 | 99.5 | 200 |
| LAV 1% Dilution | 98.5 | 1.5 | 0 | 0 | 1 | 0 | 0 | 200 |
| LAV 5% Dilution | 95 | 1.5 | 0 | 5 | 5 | 5 | 100 | 200 |
| URO Unspiked | 99 | 0.5 | 0.5 | 0 | 1 | 0 | 0 | 200 |
| PBMC | | | | | | | | |
| URO Cell Line | 0 | 0 | 0.5 | 99.5 | 100 | 99.5 | 99.5 | 200 |
| URO 1% Dilution | 98.5 | 0 | 0.5 | 1 | 1 | 1 | 100 | 200 |
| URO 5% Dilution | 94.5 | 0 | 0.5 | 5 | 5 | 5 | 100 | 200 |

### Enumeration of FISH Signals:

3p22.1 (green)/CEP 3 (red) or 10q23.2 (green)/CEP10 (red)
Normal: 2 signals for each probe
Deletion: Loss of one or both signals of 3p22.1 or 10q22.3 (green)
Monosomy: Loss of one signal for both probes (1red and 1green signal)
Polysomy: Polysomy (more than 2 signals) of each probe (3 red and 3 green or more)
Gain: Gain of one or both signals of 3p22.1 or 10q22.3 (green)

LAV or URO Probes
Normal: 2 signals for each probe
Loss: Monosomy (one signal) of one probe
Gain: Polysomy (more than 2 signals) of one probe
Abnormal: Abnormality of two probes in one cell, loss or gain

### CTC Quantitation

The number of CTCs per microliter of blood was calculated as the percentage of CACs (for a specific chromosomal probe set)×the total number of PBMCs isolated/mL of blood collected/1000. Thus, the number of CTCs with deletions or gains of 3p22.1 compared with CEP3 and the number of CTCs with deletions or gains of 10q22.3 compared with CEP10 per microliter were calculated. CTCs per microliter were calculated for the URO and LAV probe sets based on the presence of at least two chromosomal abnormalities in the biomarkers tested in each nucleus.

### Tumor Wash Specimens

Cell suspensions of tumors from 21 patients were obtained and cytospins of tumor cells were prepared. FISH was performed for the 3p22.1/CEP3, 10q22.3/CEP10, the URO and LAV probe sets and evaluated as above. Of the 21 patients from whom cell suspensions were obtained fifteen patients had adenocarcinoma, and 6 had squamous cell carcinoma. Also, 14, 3, 3, and 1 patient's had stage I, II, III, and IV NSCLC, respectively. In each case, 3 to 5 mm³ of viable tumor tissue was minced into 1× phosphate buffered saline (PBS) and vortexed with 5 mL of 1×PBS and centrifuged at 300×g. Cytospins of tumor cells were prepared and spray fixed with SAFETEX cytology spray fixative (Andwin Scientific, Woodland Hills, Calif.).

### Recovery of Lung Adenocarcinoma Cell Line Experiments

The sensitivity of the FISH-based assay to detect the presence of CTCs in peripheral blood was evaluated by performing recovery experiments in which H1299 lung adenocarcinoma cells were spiked into PBMCs isolated from healthy donors. Two separate dilution assays at 1% and 5% were performed and the spiked cell mixtures were hybridized with 3p22.1/CEP3, 10q22.3/CEP10, the LAV set, and the URO set. H1299 cells and PBMC controls were similarly hybridized and evaluated for cytogenetic abnormalities (Table 8).

### Statistical Analysis

Descriptive statistical analyses, including the Pearson χ² test, were used to test for distributional differences between the patients and controls according to categorical variables, and the Mann-Whitney test was used to determine differences in continuous variables. The Mann-Whitney test was also used to test for differences in each biomarker between the patients and controls. Simple linear regression analysis was performed to test for trends in the biomarkers by disease stage. Two-sided P values were used to determine the level of significance for each test.

To evaluate the role of each biomarker in cancer recurrence and overall survival each variable was dichotomized into two groups based on the 75th percentile of the controls for each respective outcome. Time to recurrence was defined as the number of months from the date of first treatment to that of first recurrence. Overall survival time was defined as the number of months from the date of first treatment to that of death. Patients lost to follow-up or those patients who had no recurrences or did not die prior to the end of the study were censored. The Kaplan-Meier method was used to identify any significant differences in time to recurrence and overall survival between the high and low groups for each biomarker, respectively. Biomarkers found to be significant at the 10% level in the Kaplan-Meier analyses were further evaluated using the Cox proportional hazards model adjusted for age, sex, and disease stage.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,415,723
U.S. Pat. No. 4,458,066
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,610,287
U.S. Pat. No. 5,739,169
U.S. Pat. No. 5,795,715
U.S. Pat. No. 5,801,005
U.S. Pat. No. 5,824,311
U.S. Pat. No. 5,830,880
U.S. Pat. No. 5,846,945
U.S. Pat. No. 5,889,136
U.S. Pat. No. 6,506,559
U.S. Pat. No. 6,573,099
U.S. Pat. No. 6,797,471
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2007/0218480
U.S. Patent Publn. 2009/0137412
Alberola et al., Proc. Annu. Mt. Am. Soc. Clin. Oncol., 14: A1094, 1995.
Allard et al., Clin. Cancer Res., 10:6897-6904, 2004.
Arap et al., Cancer Res., 55(6):1351-1354, 1995.
Auerbach et al., N Engl J. Med., 265: 253-267, 1961.
Austin-Ward and Villaseca, Revista Medica de Chile, 126(7):838-845, 1998.
Ayabe et al., Lung Cancer, 11(3-4): 201-208, 1994.
Bakhshi et al., Cell, 41(3):899-906, 1985.
Barkan et al., Cancer, 105:35-43, 2004.
Barkan et al., Cancer, 105:35-43, 2005.
Bonassi et al., Cancer Res., 60:1619-1625, 2000.
Bonassi et al., Genome Res., 104(1-4):376-382, 2004.
Bosher and Labouesse, Nat. Cell. Biol., 2(2):E31-E36, 2000.
Brummelkamp et al., Cancer Cell, 2:243-247, 2002.
Bubendorf et al., Am. I Clin. Pathol., 123516-123523, 2005.
Bukowski et al., Clinical Cancer Res., 4(10):2337-2347, 1998.
Caldas et al., Cancer Res., 54:3568-3573, 1994.
Carey et al., JNCI, 65: 1225-1230, 1980.
Carriaga et al., Cancer, 75: 406-421, 1995.
Cheng et al., Cancer Res., 54(21):5547-5551, 1994.
Cheon et al., Yonsei Med. 1, 34(4): 365-370, 1993.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cristofanilli et al., N. Engl. J. Med., 351:781-791, 2004.
Daniely et al., Urology, 66:1354-1359, 2005.
Davidson et al., J. Immunother., 21(5):389-398, 1998.
Ekins and Chu, Trends in Biotechnology, 17: 217-218, 1999.
EPO Appln. 320 308
EPO Appln. 329 822
Feder et al., Cancer Genet. Cytogenet., 102: 25-31, 1998.
Feinstein et al., Am. Rev. Repir. Dis., 101: 671-684, 1970.
Fenech and Crott, Mutat. Res., 504:131-136, 2002.
Fenech, Drug Discov. Today, 7:1128-1137, 2002.
Fenech, Mutat. Res., 455:81-95, 2000.
Fenech, Toxicology, 181-182:411-416, 2002.
Fire et al., Nature, 391(6669):806-811, 1998.
Fodor et al., Biochemistry, 30(33):8102-8108, 1991.
Fontanini et al., Cancer, 70(6): 1520-7, 1992.
Freifelder, In: Physical Biochemistry Applications to Biochemistry and Molecular Biology, 2nd Ed. Wm. Freeman and Co., NY, 1982.
Frohman, In: PCR Protocols: A Guide To Methods And Applications, Academic Press, N.Y., 1990.
Gangnus et al., Clin. Cancer Res., 10:3457-3464, 2004.
GB Appln. 2 202 328
Goeze et al., J. Pathol., 196:8-16, 2002.
Gough et al., Biomed. Central, 4:9:1471-2164, 2003.
Grishok et al., Science, 287:2494-2497, 2000.
Haagsman and Diemel, Comp. Biochem. Physiol. A Mol. Integr. Physiol., 129(1):91-108, 2001.
Hacia et al., Nature Genet., 14:441-449, 1996.
Hagmar et al., Cancer Res., 58:4117-4121, 1998.
Hanibuchi et al., Int. J. Cancer, 78(4):480-485, 1998.
Hellstrand et al., Acta Oncologica, 37(4):347-353, 1998.
Hirano et al., American J. Path., 144(2): 296-302, 1994.
Hirsh, Manksgaard, 1-78, 1983.
Holmstrom et al., Anal. Biochem. 209:278-283, 1993.
Hui and Hashimoto, Infection Immun., 66(11):5329-5336, 1998.
Hussussian et al., Nat. Genet., 8(1):15-21, 1994.
Ichinose et al., Cancer Res., 51(19):5392-5397, 1991.
Ihde, Curr. Prob. Cancer, 15: 65, 1991.
Ingenito et al., J. Appl. Physiol., 86(5):1702-1714, 1999.
Innis, et al., In: PCR Protocols. A guide to Methods and Application, Academic Press, Inc. San Diego, 1990.
Jiang and Katz, Diagn. Mol. Pathol., 11:47-57, 2002.
Jiang et al., Clin. Cancer Res., 115417-115424, 2005.
Jiang et al., Neoplasia, 6:623-635, 2004.
Jones, Nature, 199:280-282, 1963.
Ju et al., Gene Ther., 7(19):1672-1679, 2000.
Kamb et al., Nat. Genet., 8(1):23-26, 1994.
Kamb et al., Science, 2674:436-440, 1994.
Katz et al., Mod. Pathol., 21:950-960, 2008.
Kerr et al., Br. J. Cancer, 26(4):239-257, 1972.
Ketting et al., Cell, 99(2):133-141, 1999.
Kim et al., Cell, 87:343-355, 1996.
Korfhagen et al., Proc. Natl. Acad. Sci. USA, 93(18):9594-9599, 1996.
Kwoh et al., Proc. Natl. Acad. Sci. USA, 86:1173, 1989.
Lee et al., Biochim. Biophys. Acta, 1582:175-177, 2002.
Leversha et al., Clin. Cancer Res., 15(6):2091-7, 2009.
Li et al., Lab. Invest., 86(6):619-627, 2006.
Licciardello et al., Int. J. Radiat. Oncol. Biol. Phys., 17(3):467-476, 1989.
Liewald et al., Chirurg., 63(3): 205-10, 1992.
Lin and Avery, Nature, 402:128-129, 1999.
Liou et al., Cancer Res., 59:1481-1484, 1999.
Macchiarini et al., Proc Annu Mt. Am. Soc. Clin. Oncol., 11: A995, 1992.
Mitchell et al., Ann. NY Acad. Sci., 690:153-166, 1993.
Mitchell et al., J. Clin. Oncol., 8(5):856-869, 1990.
Mitsudomi et al., Clin. Cancer Res., 2(7): 1185-9, 1996.
Miyamoto et al., Cancer Res., 51(23pt1) 6346-50, 1991.
Montgomery et al., Proc. Natl. Acad. Sci. USA, 95:15502-15507, 1998.
Mori et al., Cancer Res., 54(13):3396-3397, 1994.
Morton et al., Arch. Surg., 127:392-399, 1992.
Mrkve et al., Anticancer Research, 13(3): 571-8, 1993.
Muller and Pantel, Breast Cancer Res., 6:258-261, 2004.
Naruke et al., J. Thorac. Cardiovas Surg., 96: 400, 1988.
Newton et al., Nucl. Acids Res. 21:1155-1162, 1993.
Nobori et al., Nature (London), 368:753-756, 1995.
Ohara et al., Proc. Natl. Acad. Sci. USA, 86:5673-5677, 1989.
Okamoto et al., Proc. Natl. Acad. Sci. USA, 91(23):11045-11049, 1994.
Orlow et al., Cancer Res, 54(11):2848-2851, 1994.
Orlow et al., Int. J. Oncol., 15(1):17-24, 1994.
Palaniyar et al., Biochem. Biophys. Res. Commun., 250(1):131-136, 1998.
Palaniyar et al., Comp. Biochem. Physiol. A Mol. Integr. Physiol., 129(1):109-127, 2001.
Pantel et al., Proc. Annu Mt. Am. Soc. Clin. Oncol., 12: A941, 1993.
Papadimitrakopoulou et al., Cancer and Metastasis Reviews, 15: 53-76, 1996.
Paul et al., Nature Biotechnol., 20:505-508, 2002.
PCT Appln. WO 00/44914
PCT Appln. WO 01/36646
PCT Appln. WO 01/68836
PCT Appln. WO 88/10315
PCT Appln. WO 89/06700
PCT Appln. WO 90/07641
PCT Appln. WO 99/32619
PCT Publn. PCT/US87/00880
PCT Publn. PCT/US89/01025,
Pease et al., Proc. Natl. Acad. Sci. USA, 91:5022-5026, 1994.
Pence et al., Archives of Surgery, 128(12): 1382-1390, 1993.
Pérez-Gil et al., Biochim. Biophys. Acta., 1408(2-3):203-217, 1998.
Pierga et al., Clin. Cancer Res., 10:1392-1400, 2004.
Pietras et al., Oncogene, 17(17):2235-2249, 1998.
Pignon et al., Hum. Mutat., 3: 126-132, 1994.
Qin et al., Proc. Natl. Acad. Sci. USA, 95(24):14411-14416, 1998.
Ramaswamy et al., Nat. Genet, 33:49-54, 2003.
Rasmussen et al., Anal. Biochem, 198:138-142, 1991.
Ravindranath and Morton, Intern. Rev. Immunol., 7: 303-329, 1991.
Rice et al., J. Thoracic Cardio. Surgery, 106(2): 201-217, 1993.
Rosenberg et al., Ann. Surg. 210(4):474-548, 1989.
Rosenberg et al., N. Engl. J. Med., 319:1676, 1988.
Running et al., BioTechniques 8:276-277, 1990.
Sahin et al., Cancer, 65(3): 530-7, 1990.
Saiki et al., J. Biochem., 116(6):1208-1211, 1994.
Sambrook et al., In: Molecular cloning: a laboratory manual, 2^{nd} Ed., Cold Spring Harbor Laboratory Press, Cold Spring Harbor, N.Y., 1989.
Serrano et al., Nature, 366:704-707, 1993.
Serrano et al., Science, 267(5195):249-252, 1995.
Sharp and Zamore, Science, 287:2431-2433, 2000.
Sharp, Genes Dev., 13:139-141, 1999.
Sher et al., Clin. Cancer Res., 11:173-179, 2005.
Shiseki et al., Genes Chromosomes Cancer, 17(2): 71-7, 1996.
Shoemaker et al., Nature Genetics, 14:450-456, 1996.
Shriver et al., Mutat. Res. 406(1):9-23, 1998.
Sieuwerts et al., J. Natl. Cancer Inst., 101:61-66, 2009.
Slaughter et al., Cancer, 6:963-968, 1953.
Smerhovsky et al., Environ. Health Perspect., 109:41-45, 2001.
Solomon et al., Science, 254:1153-1160, 1991.
Stewenius et al., Proc. Natl. Acad. Sci. USA, 102(15):5541-5546, 2005.
Sui et al., Proc. Natl. Acad. Sci. USA, 99(8):5515-5520, 2002.
Swennenhuis et al., Cytometry A, 75(6):520-7, 2009.
Tabara et al., Cell, 99(2):123-132, 1999.
Thiberville et al., Cancer Research, 55: 5133-5139, 1995a.
Thiberville et al., Int. J. Cancer, 64:371, 1995b.
Travis et al., Cancer, 75: 191-202, 1995.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
Tucker and Preston, Mutat. Res., 365:147-159, 1996.
Umegaki et al., Mutagenesis, 15:261-269, 2000.
Valdivieso et al., Proc. Annu. Mt. Am. Soc. Clin. Oncol., 13: A1121, 1994.
Vallmer et al., Hum. Pathol., 16: 247-252, 1985.
Volm et al., Versicherungsmedizin, 41(1): 2-5, 1989.
Voravud, et al., Cancer Research, 53: 2874-2883, 1993.
Walker et al., Nucleic Acids Res., 20(7):1691-1696, 1992.
Wincott et al., Nucleic Acids Res., 23(14):2677-2684, 1995.
Wistuba et al., Cancer Res., 60(7): 1949-60, 2000.
Wu et al., Anal. Chem., 70:456 A, 1998.
Wu et al., Cancer Res., 58(8):1605-8, 1998.
Wu et al., J. Thorac Oncol., 4:30-36, 2009.
Yamaoka et al., J. Japan Surgical Soc., 91(10):1608-1616, 1990.
Yanagisawa et al., Cancer Research, 56:5579-5582, 1996.
Yendamuri et al., J. Thorac Oncol., 3:979-984, 2008.
Yu et al., J. Am. Chem. Soc., 124(23):6576-6583, 2002.
Zou et al., Clinical Cancer Research 4: 1345-1355, 1998.

1. A method of detecting circulating tumor cells (CTCs) in a sample comprising:
(a) contacting said sample with a CD45 binding agent;
(b) selecting the cells based on staining for CD45;
(c) contacting the selected cells with a labeled nucleic acid probe, and detecting hybridized cells by fluorescence in situ hybridization; and
(d) analyzing a signal produced by the labels on the hybridized cells to detect the CTCs.

2. The method of claim 1, wherein the cells that are selected show positive staining for CD45.

3. The method of claim 1, wherein the cells that are selected show diminished or no staining for CD45.

4. The method of claim 1, wherein the sample is a blood sample.

5. The method of claim 4, wherein the blood sample is a buffy coat layer separated from the blood by a Ficoll-Hypaque gradient.

6. The method of claim 1, wherein the sample is a human blood sample from a patient.

7. The method of claim 6, wherein the patient is known or suspected to have cancer.

8. The method of claim 7, wherein the cancer is a form of cancer that gives rise to blood borne metastases.

9. The method of claim 8, wherein the cancer is a cancer of lung, breast, colon, prostate, pancreas, esophagus, kidney, gastro-intestinal tumors, urigenital tumors, kidney, melanomas, endocrine tumors, or sarcomas.

10. The method of claim 1, wherein the staining comprises contacting the sample with a labeled CD45 antibody.

11. The method of claim 10, wherein the label is a fluorescent label or a chromagen label.

12. The method of claim 11, wherein the fluorescently-labeled CD45 antibody is a Fluorescein isothiocyanate (FITC)-conjugated CD45 antibody.

13. The method of claim 1, wherein detecting the signal comprises using an automated fluorescence scanner.

14. The method of claim 1, wherein the probe is a 10q22-23 probe, a 3p22.1 probe, or a PI3 kinase probe.

15. The method of claim 14, wherein the probe is a UroVysion DNA probe set.

16. The method of claim 14, wherein the probe is a LaVysion DNA probe set.

17. The method of claim 14, wherein the probe is a centromeric 7/7p12 Epidermal Growth Factor (EGFR) probe.

18. The method of claim 14, wherein the probe is a combination of a commercial probe and an in-house probe.

19. The method of claim 18, wherein the combination of probes is a cep10/10q22.3 and a cep3/3p22.1.

20. The method of claim 18, wherein the combination of probes is cep7/7p22.1, a cep17, and a 9p21.3.

21. The method of claim 1, wherein selecting the cells is performed manually, by flow cytometry, by image analysis or a bright field examination using chromogen labeled probes such as DAB or AEC.

22. The method of claim 1, further comprising obtaining a patient sample.

23. A method of determining the level of circulating tumor cells (CTCs) in a sample having blood cells from a patient by:
(a) contacting said sample with a CD45 binding agent;
(b) selecting the cells based on staining for CD45;
(c) contacting the selected cells with a labeled nucleic acid probe, and detecting hybridized cells by fluorescence in situ hybridization; and
(d) analyzing a signal produced by the labels on the hybridized cells to determine the level of CTCs in the sample.

24-42. (canceled)

43. A method of detecting cancer in a patient comprising determining the level of CTCs in a biological sample containing blood cells from the patient by the method of claim 23, wherein the presence of CTCs in the sample is indicative of cancer.

44. A method of determining the level of circulating tumor cells (CTCs) in a sample having blood cells from a patient by:
(a) contacting the sample with a labeled nucleic acid probe;
(b) detecting hybridized cells by fluorescence in situ hybridization; and
(c) analyzing a signal produced by the labels on the hybridized cells to determine the level of CTCs in the sample.

45-63. (canceled)

64. A method of evaluating cancer in a patient comprising determining the level of CTCs in a biological sample containing blood cells from the patient by the method of claim 23, wherein high levels of CTCs in the sample as compared to a control is indicative of an aggressive form of cancer and/or a poor cancer prognosis.

65-69. (canceled)

70. A method of monitoring treatment of cancer in a patient comprising:
(a) determining the level of CTCs in a first sample from the patient by the method of claim 23;
(b) determining the level of CTCs in a second sample from the patient after treatment is effected by the method of claim 23; and
(c) comparing the level of CTCs in the first sample with the level of CTCs in the second sample to assess a change and monitor treatment.

71-73. (canceled)

74. A method of staging cancer in a patient comprising determining the level of CTC expression in a biological sample containing blood cells from the patient by the method of claim 23, wherein a higher level of CTC in the sample as compared to a control is indicative of a more advanced stage of cancer and a lower level of CTC in the sample as compared to a control is indicative of a less advanced stage of cancer.

75-86. (canceled)

87. The method of claim 74, wherein the method is used to refine the staging of cancer after treatment has started.

88. A method of staging cancer in a patient comprising determining the level of CTC expression in a biological sample containing blood cells from the patient by the method of claim 23, wherein a higher or lower level of expression of a gene of interest in the sample as compared to a control is indicative of a more advanced stage of cancer and a lower level of expression of the gene of interest in the sample as compared to a control is indicative of a less advanced stage of cancer.
